(19) United States
(12) Patent Application Publication
(10) Pub. No.: US 2007/0020737 A1
(43) Pub. Date: Jan. 25, 2007

(54) Hyaluronan synthases and methods of making and using same

(76) Inventors: Philip E. Pummill, Oklahoma City, OK (US); Paul L. DeAngelis, Edmond, OK (US); Paul H. Weigel, Edmond, OK (US)

Correspondence Address: DUNLAP, CODDING & ROGERS P.C., PO BOX 16370, OKLAHOMA CITY, OK, 73113, US

(21) Appl. No.: 11/495,989

(22) Filed: Jul. 28, 2006

Related U.S. Application Data
(63) Continuation-in-part of application No. 11/444,093, filed on May 31, 2006.
Application No. 11/444,093 is a continuation of application No. 10/309,560, filed on Dec. 3, 2002, now Pat. No. 7,094,581.
Application No. 11/444,093 is a continuation-in-part of application No. 10/011,771, filed on Dec. 11, 2001, now Pat. No. 7,109,011.
(60) Provisional application No. 60/704,003, filed on Jul. 29, 2005.
(60) Provisional application No. 60/336,105, filed on Dec. 3, 2001.

(51) Int. Cl. C12P 19/28; C07H 21/04; C08B 37/00; C12P 21/06; C12N 9/24; C12N 15/74; C12N 1/21

(52) U.S. Cl. 435/85; 435/69.1; 435/200; 435/252.33; 435/471; 536/23.2; 536/53

(57) ABSTRACT

A functionally active hyaluronan synthase having at least one modified amino acid residue therein as compared to a corresponding functionally active native hyaluronan synthase such that the functionally active hyaluronan synthase has an altered enzymatic activity as compared to the corresponding functionally active native hyaluronan synthase is disclosed. The altered enzymatic activity may result in increased or decreased activity when compared to the corresponding native hyaluronan synthase, or the altered enzymatic activity may result in production of hyaluronan having an average molecular mass that is greater than or less than an average molecular mass of hyaluronan produced by the corresponding native hyaluronan synthase. Methods of producing hyaluronic acid utilizing a recombinant host cell having an expression construct encoding the functionally active hyaluronan synthase with altered enzymatic activity are also disclosed.

### CROSS-REFERENCE TO RELATED APPLICATIONS

This application claims benefit under 35 U.S.C. 119(e) of U.S. Ser. No. 60/704,003, filed Jul. 29, 2005, the contents of which are hereby expressly incorporated herein by reference.

This application is also a continuation-in-part of U.S. Ser. No. 11/444,093, filed May 31, 2006; which is a continuation of U.S. Ser. No. 10/309,560, filed Dec. 3, 2002; which claims benefit under 35 U.S.C. 119(e) of U.S. Ser. No. 60/336,105, filed Dec. 3, 2001; the contents of which are hereby expressly incorporated herein by reference.

Said U.S. Ser. No. 10/309,560 is also a continuation-in-part of U.S. Ser. No. 10/011,771, filed Dec. 11, 2001; the contents of which are hereby expressly incorporated herein by reference.

### STATEMENT REGARDING FEDERALLY SPONSORED RESEARCH OR DEVELOPMENT

This application was supported in part by a grant from the National Institutes of Health (GM35978). The United States Government may have rights in and to this application by virtue of this funding.

### BACKGROUND OF THE INVENTION

1. Field of the Invention

The present invention relates to nucleic acid segments having coding regions encoding enzymatically active hyaluronate synthase (HAS), and to the use of these nucleic acid segments in the preparation of recombinant cells which produce hyaluronate synthase and its hyaluronic acid product. Hyaluronate is also known as hyaluronic acid or hyaluronan. More particularly, but not by way of limitation, the nucleic acid segments disclosed and claimed herein have at least one mutation as compared to the native nucleic acid segments such that the at least one mutation results in kinetic or enzymatic changes/modifications to the resulting enzyme.

2. Brief Description of the Related Art

The incidence of streptococcal infections is a major health and economic problem worldwide, particularly in developing countries. One reason for this is due to the ability of Streptococcal bacteria to grow undetected by the body's phagocytic cells, i.e., macrophages and polymorphonuclear cells (PMNs). These cells are responsible for recognizing and engulfing foreign microorganisms. One effective way the bacteria evades surveillance is by coating themselves with polysaccharide capsules, such as a hyaluronic acid (HA) capsule. The structure of HA is identical in both prokaryotes and eukaryotes.

Since HA is generally nonimmunogenic, the encapsulated bacteria do not elicit an immune response and are therefore not targeted for destruction. Moreover, the capsule exerts an antiphagocytic effect on PMNs in vitro and prevents attachment of Streptococcus to macrophages. Precisely because of this, in Group A and Group C Streptococci, the HA capsules are major virulence factors in natural and experimental infections. Group A Streptococcus are responsible for numerous human diseases including pharyngitis, impetigo, deep tissue infections, rheumatic fever and a toxic shock-like syndrome. The Group C Streptococcus equisimilis is responsible for osteomyelitis, pharyngitis, brain abscesses, and pneumonia.

Structurally, HA is a high molecular weight linear polysaccharide of repeating disaccharide units consisting of N-acetylglucosamine (GlcNAc) and glucuronic acid (GlcUA). The number of repeating disaccharides in an HA molecule can exceed 30,000, a Mᵣ>10⁷. HA is the only glycosaminogylcan synthesized by both mammalian and bacterial cells, particularly Groups A and C Streptococci and Type A Pasteurella multocida. These strains make HA which is secreted into the medium as well as HA capsules. The mechanism by which these bacteria synthesize HA is of broad interest medicinally since the production of the HA capsule is a very efficient and clever method that bacteria use to evade surveillance by the immune system. Additionally, organic or inorganic molecules coated with HA have properties allowing them to escape detection and destruction by a host's immune system.

HA is synthesized by mammalian and bacterial cells by the enzyme hyaluronate synthase which has been localized to the plasma membrane. It is believed that the synthesis of HA in these organisms is a multi-step process. Initiation involves binding of an initial precursor, UDP-GlcNAc or UDP-GlcUA. This is followed by elongation which involves alternate addition of the two sugars to the growing oligosaccharide chain. The growing polymer is extruded across the plasma membrane region of the cell and into the extracellular space.

HA has been identified in virtually every tissue in vertebrates and has achieved widespread use in various clinical applications, most notably and appropriately as an intra-articular matrix supplement and in eye surgery. The scientific literature has also shown a transition from the original perception that HA is primarily a passive structural component in the matrix of a few connective tissues and in the capsule of certain strains of bacteria to a recognition that this ubiquitous macromolecule is dynamically involved in many biological processes: from modulating cell migration and differentiation during embryogenesis to regulation of extracellular matrix organization and metabolism to important roles in the complex processes of metastasis, wound healing, and inflammation. Further, it is becoming clear that HA is highly metabolically active and that cells focus much attention on the processes of its synthesis and catabolism. For example, the half-life of HA in tissues ranges from 1 to 3 weeks in cartilage to <1 day in epidermis. HA is also used in numerous technical applications (e.g., lubricating compounds), cosmetics and neutraceuticals.

It is now clear that a single protein utilizes both sugar substrates to synthesize HA, i.e., that HA synthases are single enzymes that have dual catalytic properties. The abbreviation HAS, for HA synthase, has gained widespread support for designating this class of enzymes. Markovitz et al. (1959) successfully characterized the HAS activity from Streptococcus pyogenes and discovered the enzymes's membrane localization and its requirements for sugar nucleotide precursors and Mg²⁺. Prehm (1983) found that elongating HA, made by B6 cells, was digested by hyaluronidase added to the medium and proposed that HAS resides at the plasma membrane. Philipson and Schwartz (1984) also showed that HAS activity cofractionated with plasma membrane markers in mouse oligodendroglioma cells.

HAS assembles high Mᵣ HA that is simultaneously extruded through the membrane into the extracellular space (or to make the cell capsule in the case of bacteria) as glycosaminoglycan synthesis proceeds. This mode of biosynthesis is unique among macromolecules since nucleic acids, proteins, and lipids are synthesized in the nucleus, endoplasmic reticulum/Golgi, cytoplasm, or mitochondria. The extrusion of the growing chain into the extracellular space also allows for unconstrained polymer growth, thereby achieving the exceptionally large size of HA, whereas confinement of synthesis within a Golgi or post-Golgi compartment limits the overall amount or length of the polymers formed. High concentrations of HA within a confined lumen may also create a high viscosity environment that might be deleterious for other organelle functions.

Several studies have attempted to solubilize, identify, and purify HAS from strains of Streptococci that make a capsular coat of HA as well as from eukaryotic cells. Although the streptococcal and murine oligodendroglioma enzymes were successfully detergent-solubilized and studied, efforts to purify an active HAS for further study or molecular cloning remained unsuccessful for decades. Prehm and Mausolf (1986) used periodate-oxidized UDP-GlcUA or UDP-GlcNAc to affinity label a protein of ˜52 kDa in streptococcal membranes that co-purified with HAS. This led to a report claiming that the Group C streptococcal HAS had been cloned, which was unfortunately erroneous. This study failed to demonstrate expression of an active synthase and may have actually cloned a peptide transporter. Triscott and van de Rijn (1986) used digitonin to solubilize HAS from streptococcal membranes in an active form. Van de Rijn and Drake (1992) selectively radiolabeled three streptococcal membrane proteins of 42, 33, and 27 kDa with 5-azido-UDP-GlcUA and suggested that the 33-kDa protein was HAS. As shown later, however, HAS actually turned out to be the 42-kDa protein.

Despite these efforts, progress in understanding the regulation and mechanisms of HA synthesis was essentially stalled, since there were no molecular probes for HAS mRNA or HAS protein. A major breakthrough occurred in 1993 when DeAngelis et al. (1993a and 1993b) reported the molecular cloning and characterization of the Group A streptococcal gene encoding the protein HasA. This gene was known to be part of an operon required for bacterial HA synthesis, although the function of this protein, which is now designated as spHAS (the S. pyogenes HAS), was unknown. spHAS was subsequently proven to be responsible for HA elongation (DeAngelis and Weigel, 1994) and was the first glycosaminoglycan synthase identified and cloned and then successfully expressed. The S. pyogenes HA synthesis operon encodes two other proteins. HasB is a UDP-glucose dehydrogenase, which is required to convert UDP-glucose to UDP-GlcUA, one of the substrates for HA synthesis. HasC is a UDP-glucose pyrophosphorylase, which is required to convert glucose 1-phosphate and UTP to UDP-glucose. Co-transfection of both hasA and hasB genes into either acapsular Streptococcus strains or Enteroccus faecalis conferred them with the ability to synthesize HA and form a capsule. This provided the first strong evidence that spHAS (hasA) was an HA synthase. The spHAS was identified and is disclosed in detail in U.S. Ser. No. 09/146,893, filed Sep. 3, 1998, now U.S. Pat. No. 6,455,304, the contents of which are expressly incorporated herein in their entirety by reference.

The elusive HA synthase gene was finally cloned by a transposon mutagenesis approach, in which an acapsular mutant Group A strain was created containing a transposon interruption of the HA synthesis operon. Known sequences of the transposon allowed the region of the junction with streptococcal DNA to be identified and then cloned from wild-type cells. The encoded spHAS was 5-10% identical to a family of yeast chitin synthases and 30% identical to the Xenopus laevis protein DG42 whose function was unknown at the time (developmentally expressed during gastrulation), DeAngelis, et al. 1994. DeAngelis and Weigel (1994) expressed the active recombinant spHAS in Escherichia coli and showed that this single purified gene product synthesizes high Mᵣ HA when incubated in vitro with UDP-GlcUA and UDP-GlcNAc, thereby showing that both glycosyltransferase activities required for HA synthesis are catalyzed by the same protein, as first proposed in 1959. Utilizing the knowledge that (i) spHAS was a dual action single enzyme, and (ii) the areas of sequence homology between the spHAS, chitin synthase, and DG42, the almost simultaneous identification of eukaryotic HAS cDNAs in 1996 by four laboratories, further strengthened the inventor's protein hypothesis that HAS is a multigene family encoding distinct isozymes. Two genes (HAS1 and HAS2) were quickly discovered in mammals, and a third gene HAS3 was later discovered. A second streptococcal seHAS or Streptococcus equisimilis hyaluronate synthase, was identified and is disclosed in detail in U.S. Ser. No. 09/469,200, filed Dec. 21, 1999, the contents of which are expressly incorporated herein in their entirety by reference. The seHAS protein has a high level of identity (approximately 70 percent) to the spHAS enzyme. This identity, however, is interesting because the seHAS gene does not cross-hybridize to the spHAS gene.

Membranes prepared from E. coli expressing recombinant seHAS synthesize HA when both substrates are provided. The results confirm that the earlier report of Lansing et al. (1993) claiming to have cloned the Group C HAS was wrong. Unfortunately, several studies have employed antibodies to this uncharacterized 52-kDa streptococcal protein to investigate what was believed to be eukaryotic HAS.

Itano and Kimata (1996a) used expression cloning in a mutant mouse mammary carcinoma cell line, unable to synthesize HA, to clone the first putative mammalian HAS cDNA (mmHAS1). Subclones defective in HA synthesis fell into three separate classes that were complementary for HA synthesis in somatic cell fusion experiments, suggesting that at least three proteins are required. Two of these classes maintained some HA synthetic activity, whereas one showed none. The latter cell line was used in transient transfection experiments with cDNA prepared from the parental cells to identify a single protein that restored HA synthetic activity. Sequence analyses revealed a deduced primary structure for a protein of ˜65 kDa with a predicted membrane topology similar to that of spHAS. mmHAS1 is 30% identical to spHAS and 55% identical to DG42. The same month this report appeared, three other groups submitted papers describing cDNAs encoding what was initially thought to be the same mouse and human enzyme. However, through an extraordinary circumstance, each of the four laboratories had discovered a separate HAS isozyme in both species.

Using a similar functional cloning approach to that of Itano and Kimata, Shyjan et al. (1996) identified the human homolog of HAS1. A mesenteric lymph node cDNA library was used to transfect murine mucosal T lymphocytes that were then screened for their ability to adhere in a rosette assay. Adhesion of one transfectant was inhibited by antisera to CD44, a known cell surface HA-binding protein, and was abrogated directly by pretreatment with hyaluronidase. Thus, rosetting by this transfectant required synthesis of HA. Cloning and sequencing of the responsible cDNA identified hsHAS1. Itano and Kimata (1996b) also reported a human HAS1 cDNA isolated from a fetal brain library. The hsHAS1 cDNAs reported by the two groups, however, differ in length; they encode a 578 or a 543 amino acid protein, respectively. HAS activity has only been demonstrated for the longer form.

Based on the molecular identification of spHAS as an authentic HA synthase and regions of near identity among DG42, spHAS, and NodC (a β-GlcNAc transferase nodulation factor in Rhizobium), Spicer et al. (1996) used a degenerate RT-PCR approach to clone a mouse embryo cDNA encoding a second distinct enzyme, which is designated mmHAS2. Transfection of mmHAS2 cDNA into COS cells directed de novo production of an HA cell coat detected by a particle exclusion assay, thereby providing strong evidence that the HAS2 protein can synthesize HA. Using a similar approach, Watanabe and Yamaguchi (1996) screened a human fetal brain cDNA library to identify hsHAS2. Fulop et al. independently used a similar strategy to identify mmHAS2 in RNA isolated from ovarian cumulus cells actively synthesizing HA, a critical process for normal cumulus oophorus expansion in the pre-ovulatory follicle. Cumulus cell-oocyte complexes were isolated from mice immediately after initiating an ovulatory cycle, before HA synthesis begins, and at later times when HA synthesis is just beginning (3 h) or already apparent (4 h). RT-PCR showed that HAS2 mRNA was absent initially but expressed at high levels 34 h later suggesting that transcription of HAS2 regulates HA synthesis in this process. Both mmHAS2 and hsHAS2 are 552 amino acids in length and are 98% identical. mmHAS1 is 583 amino acids long and 95% identical to hsHAS1, which is 578 amino acids long.

Spicer et al. (1998) used a PCR approach to identify a third HAS gene in mammals. The mmHAS3 protein is 554 amino acids long and 59, 71, 56, and 28% identical, respectively, to mmHAS1, mmHAS2, DG42, and spHAS. Spicer et al. have also localized the three human and mouse genes to three different chromosomes (HAS1 to hsChr 19/mmChr 17; HAS2 to hsChr 8/mmChr 15; HAS3 to hsChr 16/mmChr 8). Localization of the three HAS genes on different chromosomes and the appearance of HA throughout the vertebrate class suggest that this gene family is ancient and that isozymes appeared by duplication early in the evolution of vertebrates. The high identity (˜30%) between the bacterial and eukaryotic HASs also suggests that the two had a common ancestral gene. Perhaps primitive bacteria usurped the HAS gene from an early vertebrate ancestor before the eukaryotic gene products became larger and more complex. Alternatively, the bacteria could have obtained a larger vertebrate HAS gene and deleted regulatory sequences nonessential for enzyme activity.

The discovery of X. laevis DG42 by Dawid and co-workers played a significant role in these recent developments, even though this protein was not known to be an HA synthase. Nonetheless, that DG42 and spHAS were 30% identical was critical for designing oligonucleotides that allowed identification of mammalian HAS2. Ironically, definitive evidence that DG42 is a bona fide HA synthase was reported only after the discoveries of the Mammalian isozymes, when DeAngelis and Achyuthan (1996) expressed the recombinant protein in yeast (an organism that cannot synthesize HA) and showed that it synthesizes HA when isolated membranes are provided with the two substrates. Meyer and Kreil (1996) also showed that lysates from cells transfected with cDNA for DG42 synthesize elevated levels of HA. Now that its function is known, DG42 can, therefore, be designated xlHAS.

There are common predicted structural features shared by all the HAS proteins, including a large central domain and clusters of 2-3 transmembrane or membrane-associated domains at both the amino and carboxyl ends of the protein. The central domain, which comprises up to ˜88% of the predicted intracellular HAS protein sequences, probably contains the catalytic regions of the enzyme. This predicted central domain is 264 amino acids long in spHAS (63% of the total protein) and 307-328 residues long in the eukaryotic HAS members (54-56% of the total protein). The exact number and orientation of membrane domains and the topological organization of extracellular and intracellular loops has been determined experimentally for spHAS and will be described in detail herein with respect to FIG. 14.

spHAS is a HAS family member that has been purified and partially characterized. Initial studies using spHAS/alkaline phosphatase fusion proteins indicate that the N terminus, C terminus, and the large central domain of spHAS are, in fact, inside the cell. spHAS has 6 cysteines, whereas HAS1, HAS2, and HAS3 have 13, 14 and 14 Cys residues, respectively. Two of the 6 Cys residues in spHAS are conserved and identical in HAS1 and HAS2. Only one conserved Cys residue is found at the same position (Cys-225 in spHAS) in all the HAS family members. This may be an essential Cys whose modification by sulfhydryl poisons partially inhibits enzyme activity. The possible presence of disulfide bonds or the identification of critical Cys residues needed for any of the multiple HAS functions noted below has not yet been elucidated for any members of the HAS family.

In addition to the proposed unique mode of synthesis at the plasma membrane, the HAS enzyme family is highly unusual in the large number of functions required for the overall polymerization of HA. At least six discrete activities are present within the HAS enzyme: binding sites for each of the two different sugar nucleotide precursors (UDP-GlcNAc and UDP-GlcUA), two different glycosyltransferase activities, one or more binding sites that anchor the growing HA polymer to the enzyme (perhaps related to a B-X₇-B motif), and a ratchet-like transfer mechanism that moves the growing polymer one or two sugars at a time. This later activity is likely coincident with the stepwise advance of the polymer through the membrane. All of these functions, and perhaps others as yet unknown, are present in a relatively small protein ranging in size from 417 (seHAS) to 588 (xlHAS) amino acids.

Although all the available evidence supports the conclusion that only the spHAS protein is required for HA biosynthesis in bacteria or in vitro, it is possible that the larger eukaryotic HAS family members are part of multicomponent complexes. Since the eukaryotic HAS proteins are ˜40% larger than spHAS, their additional protein domains could be involved in more elaborate functions, such as intracellular trafficking and localization, regulation of enzyme activity, and mediating interactions with other cellular components.

The unexpected finding that there are multiple vertebrate HAS genes encoding different synthases strongly supports the emerging consensus that HA is an important regulator of cell behavior and not simply a structural component in tissues. Thus, in less than six months, the field moved from one known, cloned HAS (spHAS) to recognition of a multigene family that promises rapid, numerous, and exciting future advances in our understanding of the synthesis and biology of HA.

For example, disclosed herein are the nucleotide sequences of HAS genes as well as the amino acid sequences encoded therein from Streptococcus equisimilis (SEQ ID NOS: 1 and 2, respectively), Streptococcus pyogenes (SEQ ID NOS:3 and 4, respectively), Streptococcus uberis (SEQ ID NOS:5 and 6, respectively), Pasteurella multocida (SEQ ID NOS:7 and 8, respectively), Xenopus laevis (SEQ ID NOS:9 and 10, respectively), Paramecium bursaria Chlorella virus (PBCV-1; SEQ ID NOS:11 and 12, respectively), and Sulfolobus solfataricus (SEQ ID NOS:13 and 14, respectively). The presence of hyaluronan synthase in these systems and the purification and use of the hyaluronan synthase from these different systems indicates an ability to purify and isolate nucleic acid sequences encoding enzymatically active hyaluronan synthase in many different prokaryotic and viral sources, indeed, from microbial sources in general.

Group C Streptococcus equisimilis strain D181 synthesizes and secretes hyaluronic acid (HA). Investigators have used this strain and Group A Streptococcus pyogenes strains, such as S43 and A111, to study the biosynthesis of HA and to characterize the HA-synthesizing activity in terms of its divalent cation requirement, precursor (UDP-GlcNAc and UDP-GlcUA) utilization, and optimum pH.

Traditionally, HA has been prepared commercially by isolation from either rooster combs or extracellular media from Streptococcal cultures. One method which has been developed for preparing HA is through the use of cultures of HA-producing Streptococcal bacteria. U.S. Pat. No. 4,517,295, the contents of which are herein incorporated by reference in their entirety, describes such a procedure wherein HA-producing Streptococci are fermented under anaerobic conditions in a CO₂-enriched growth medium. Under these conditions, HA is produced and can be extracted from the broth. It is generally felt that isolation of HA from rooster combs is laborious and difficult, since one starts with HA in a less pure state. The advantage of isolation from rooster combs is that the HA produced is of higher molecular weight. However, preparation of HA by bacterial fermentation is easier, since the HA is of higher purity to start with. Usually, however, the molecular weight of HA produced in this way is smaller than that from rooster combs. Additionally, HA prepared by Streptococcal fermentation oftentimes elicits immune responses as does HA obtained from rooster combs. Therefore, a technique that allows for the production of high molecular weight HA by bacterial fermentation would be a distinct improvement over existing procedures.

As mentioned previously, high molecular weight HA has a wide variety of useful applications—ranging from cosmetics to eye surgery. Due to its potential for high viscosity and its high biocompatibility, HA finds particular application in eye surgery as a replacement for vitreous fluid. HA has also been used to treat racehorses for traumatic arthritis by intra-articular injections of HA, in shaving cream as a lubricant, and in a variety of cosmetic products due to its physiochemical properties of high viscosity and its ability to retain moisture for long periods of time. In fact, in August of 1997 the U.S. Food and Drug Agency approved the use of high molecular weight HA in the treatment of severe arthritis through the injection of such high molecular weight HA directly into the affected joints. In general, the higher the molecular weight of HA that is employed the better. This is because HA solution viscosity increases with the average molecular weight of the individual HA polymer molecules in the solution. Unfortunately, very high molecular weight HA, such as that ranging up to 10⁷, has been difficult to obtain by currently available isolation procedures. The recombinant methods of production disclosed herein, however, allow for the production of HA having an average molecular mass of up to 10⁷ and greater.

To address these or other difficulties, there is a need for new methods and constructs that can be used to produce HA having one or more improved properties such as greater purity, ease of preparation or desired product size. In particular, there is a need to develop methodology for the production of larger amounts of relatively high molecular weight and relatively pure HA than is currently commercially available. There is yet another need to be able to develop methodology for the production of HA having a modified size distribution (HA_{Δsize}) as well as HA having a modified structure (HA_{Δmod}).

Although the streptococcal HA synthases are relatively small at <49 kDa, they mediate at least six discrete functions: the ability to bind two different sugar nucleotide precursors, to catalyze two distinct glycosyltransferase reactions, to bind the HA acceptor polymer and to translocate the growing HA chain through the enzyme and the cell membrane.

All recombinant HASs, either from vertebrates or prokaryotes, have been shown to synthesize high molecular weight HA in vitro. The class I HAS proteins likely have essentially identical topological organizations in their N-terminal regions, which are highly homologous with spHAS, the only HAS whose membrane topology has been determined experimentally.

There are six Cys residues in spHAS, four of which are conserved perfectly in seHAS and suHAS (FIG. 1); both of these latter enzymes have only four Cys residues (Kumari and Weigel, 1997; Ward et al., 2001). These four Cys residues in turn are generally conserved among the three vertebrate HAS isoenzymes (Weigel et al., 1997, and FIG. 1). However, to date the involvement of one or more of these conserved Cys residues in enzyme activity or disulfide bond formation has not been determined.

The present invention addresses one or more shortcomings in the art. Using recombinant DNA technology, methods of producing enzymatically active HAS having at least one mutation therein (as compared to the native enzyme) is disclosed and claimed in conjunction with the preparation of recombinant cells which produce HAS and its hyaluronic acid product.

### BRIEF DESCRIPTION OF THE DRAWINGS

FIG. 1. General conservation of four cysteines in seHAS within the Class I HAS protein family. The HAS protein sequences (and their accession numbers) shown are: Streptococcus equisimilis (seHAS, AAB87874, SEQ ID NO:2); Streptococcus uberis (suHAS, CAB46918, SEQ ID NO:6); Streptococcus pyogenes (spHAS, AAA17981, SEQ ID NO:4); chicken (ggHAS2, AF106940_{—}1); mouse (mmHAS1, BAA11654; mmHAS2, AAC53309; mmHAS3, AAC53128); human (hsHAS1, NP_{—}001514; hsHAS2, NP_{—}005319; hsHAS3, AF232772_{—}1); rabbit (ocHAS2, BAB63264; ocHAS3, BAB63265); bovine (btHAS2, CM06239); rat (rnHAS2, NP_{—}037285); chlorella virus (cvHAS2, AF113757_{—}1) and frog (xlHAS1, AF106940). The sequenceswere aligned using the DNAsis multiple alignment program (v4.0). Cys residues including the four in seHAS that are conserved in all three streptococcal enzymes, are in boldface. The bars highlight the sequence regions of these four conserved Cys residues within the larger HAS family. Residues that are identical in the three HASs and in some of the other family members are highlighted in dark gray. Residues in seHAS conserved among all other HAS family members are highlighted in light gray. Conserved residues that are within the active sites of all β-glycosyltransferases are indicated by a dot.

FIG. 2. Effect of NEM concentration and incubation time on the activity of seHAS and spHAS. Panel A: E. coli membranes containing recombinant seHAS or spHAS were incubated at 4° C. for 1 h with Phosphate Buffered Saline (PBS) alone (minus N-ethylmaleimide (NEM) control) or PBS containing different concentrations of NEM. The unreacted NEM was quenched by addition of dithioerythritol (DTE) to a final concentration of 1-6 mM and the samples were assayed for HAS activity as described hereinafter. Panel B: The effect of incubation time on seHAS and spHAS activity was assessed by incubating the membranes with 5 mM NEM at 4° C. for the indicated times. Aliquots were removed into assay buffer containing 5 mM DTE, and HAS activities were determined. HAS activity in control untreated membranes was stable for 1 h at 4° C. The inhibition of HAS activity is expressed as percent relative to the controls.

FIG. 3. Effect of NEM or sodium arsenite treatment on the utilization of UDP-GlcUA and UDP-GlcNAc by wild-type seHAS. E. coi membranes containing seHAS protein were incubated at 4° C. for 1 h in PBS containing 5 mM NEM or 10 mM Sodium Arsenite (SodArs), and the control membranes were incubated with PBS alone. Michaelis-Menten constants (Kₘ) were calculated from the activities of seHAS at varying concentrations of UDP-GlcUA or UDP-GlcNAc.

FIG. 4. Relative enzyme activities of the Cys-to-Ala or Cys-to-Ser single Cys-mutants of seHAS. Membranes from E. coli (SURE) cells expressing either wild-type seHAS or the indicated single Cys-mutants of seHAS were assayed for HAS activity under linear conditions with respect to time and protein concentration, and the amount of HAS protein expressed in each membrane preparation was determined as described hereinafter. The normalized seHAS specific activities were calculated as nmol of UDP-GlcUA incorporated per pmol of HAS per hour. The specific activities of seHAS mutants are given as a percent relative to wild-type activity as 100%.

FIG. 5. Relative enzyme activities of the Cys-to-Ala multiple Cys-mutants of seHAS. Membranes expressing wild-type seHAS, the indicated multiple Cys-mutants of seHAS or the seHAS^{Cys-null} were assayed and normalized as described in FIG. 4.

FIG. 6. Substrates protect triple Cys-mutants of seHAS from inactivation by NEM. E. coli membranes containing the indicated triple Cys-mutant of seHAS (each with a single remaining Cys residue) were treated at 4 C for 10 min with no additions (control; 100% values) or with 5 mM NEM alone and in the presence of either UDP, UDP-GlcUA or UDP-GlcNAc. Unreacted NEM was then quenched and HAS activity was determined. Results are not shown for the mutant containing only Cys³⁶⁷, since NEM inactivation of this mutant is ˜10% and all values were essentially identical.

FIG. 7. Relative sizes of HA synthesized by wild-type seHAS and the Cys-mutants of seHAS. E. coli membranes containing wild-type or the 19 Cys-mutants of seHAS were incubated with UDP-[¹⁴C]GlcUA and the othercomponents described hereinafterforthe assay of HAS activity. The ¹⁴C-labeled HA products were then recovered and analyzed by agarose gel electrophoresis and autoradiography as described hereinafter. The molecular weight markers used were the indicated DNA fragments of defined length (kb). A 7 kb DNA fragment corresponds to an HA molecular weight of approximately 10⁶ (32). SeHAS variants shown are as follows. Panel A: Lane 1, C226A; Lane 2, C262A; Lane 3, C281A; Lane 4, C367A; Lane 5, wild-type; lane 6, C226S; Lane 7, C262S; Lane 8, C281S; Lane 9, C367S. Panel B: Lane 1, C(226,262)A; Lane 2, C(226,281)A; Lane 3, C(226,367)A; Lane 4, C(262,281)A; Lane 5, C(262,367)A; Lane 6, C(281,367)A; Lane 7, wild-type; Lane8, (Δ3C)C²⁶²; Lane 9, (Δ3C)C²⁸¹; Lane 10, (Δ3C)C³⁶⁷; Lane 11, seHAS^{Cys-null}; Lane 12, (Δ3C)C²²⁶.

FIG. 8. Reactivity of ¹⁴C-NEM with the Cys-to-Ala double mutants of seHAS. E. coli membranes containing wild-type or double Cys-mutants of seHAS were incubated in two separate experiments (panels A and B) with 2.5 mM ¹⁴C-NEM (8×10⁶ dpm) at 4° C. for 10 min. The excess of ¹⁴C-NEM was quenched by addition of 40 mM DTE and incubation for 5 min at 4° C. Trichloroacetic acid was added to a final concentration of 10%, and the samples were incubated at 4° C. overnight. The membrane pellet was washed by centrifugation 3 times with 5% TCA, suspended in 20 μl of Laemmli sample buffer (33) and neutralized with sodium hydroxide. The samples were heated at 95° C. for 3 minutes and subjected to SDS-PAGE. The gels were processed and analyzed as described hereinafter.

FIG. 9. MALDI-TOF mass spectrographs of seHAS-His₆ derivatives covalently modified by a sulfhydryl reagent. Wild-type seHAS-His₆ (panel A) or seHAS-His_{Cys-null} (panel B) were incubated with (the upper traces in each panel) or without (lower traces in each panel) biotin-PEO-maleimide, and the eluted proteins were then prepared for mass analysis as described hereinafter. The predicted mass-to-charge ratios for covalent adducts containing 2, 3 or 4 biotin-PEO-maleimide groups perwild-type enzyme molecule (in parentheses) and the observed centroid mass-to-charge ratios are indicated above the peaks. The predicted m/z ratio for the (MH)⁺ ion of unmodified seHAS^{Cys-null}-His₆ (with four Ala residues replacing the four Cys residues) is 48,473.1.

FIG. 10. Kinetic analysis of UDP-GlcUA utilization by Cys-mutants of spHAS. Membranes prepared from cells expressing the indicated spHAS mutant were assayed as described hereinafter to assess the Michaelis-Menton constants for UDP-GlcUA: wildtype (□), C(124,366,402)A (▾), C(124,261,280,366,402)A (▴), and the Cys-null mutant (602 ).

FIG. 11. Hill analysis of UDP-GlcNAc utilization by Cys-mutants of spHAS. Hill plots of data obtained from Kₘ assays of wildtype and several mutant spHAS proteins, performed as in FIG. 10, demonstrate that the cooperative nature of UDP-GlcNAc utilization is not affected by alteration of Cys residues. The spHAS variants shown are: wildtype (□), C(124,366,402)A (▾), C(124,261,280,366,402)A (▴), and the Cys-null mutant (∘).

FIG. 12. Inhibition of wildtype and Cys-mutants of spHAS by NEM. The activity of the indicated spHAS enzymes in membranes was assessed after pretreatment with (gray bars) or without (black bars) 20 mM NEM at room temperature for 90 min. Wildtype and many of the Cys-mutant variants of spHAS with multiple Cys residues mutated are still sensitive to NEM inhibition. The mutant containing only one Cys residue at Cys²²⁵ and the Cys-null mutant were not sensitive to NEM inhibition.

FIG. 13. MALDI-TOF mass spectrographs of spHAS-His₆ covalently modified by a sulfhydryl reagent. Wildtype spHAS-His₆ (panel A) or the Cys-null mutant of spHAS-His₆ (panel B) were bound to Ni⁺²-NTA resin, washed and incubated for 2 h at 4° C. with (the upper traces in each panel) or without (lower traces in each panel) 10 mg/ml biotin-PEO-maleimide. The columns were washed and the proteins were then eluted and prepared for mass analysis as described hereinafter. The centroid mass-to-charge ratios are indicated above the observed peaks and the predicted mass-to-charge ratios for covalent adducts containing 2, 3, 4, 5 or 6 biotin-PEO-maleimide groups per wildtype enzyme molecule are indicated in parentheses. The predicted m/z ratio for the (MH)⁺ ion of unmodified spHAS^{Cys-null}-His₆ (with six Ala residues replacing the six Cys residues) is 48,484.4.

FIG. 14. Alignment of seHAs, spHAS and suHAS sequences (FIG. 14A), and topological organization of spHAS and probably all Class I HASs (FIGS. 14B and C). Membrane domains (MDs) are numbered 1-6 starting from the N-terminus. FIG. 14B: the orientation of MD1 through MD6 (white numbers) in seHAS, and position number of amino acids at the cytoplasmic junctions for each MD are indicated. K48 and E327 are in white circles within MD2 and MD4, respectively; the suggested interaction between the two is indicated by the dashed double-arrow. FIG. 14C: a possible organization of MD1-MD6 in which MD2 and MD4 are in contact (with K48 and E327 indicated in the white box). The black area within the MD cluster denotes the putative intramolecular pore, through which a growing HA chain is translocated across the membrane. In both panels, the cell interior is at the bottom.

FIG. 15. seHAS mutants at K48 and E327. Sequence alignments show K48 of seHAS within MD2 (using the nomenclature in FIG. 14). Note that seHAS and spHAS contain 417 and 419 amino acids, respectively, and their numbering is not identical. The analogous position in spHAS is also Lys, and in the eukaryotic HASs this is a conserved polar residue: Gin, which could be involved in one or more H-bonds. MD4 contains an absolutely conserved Glu residue in the Class I HAS family, which is E327 in seHAS.

FIG. 16. Expression and quantitation of recombinant seHAS variants at K48 and E327 in E. coli membranes. Membranes were prepared from cells expressing wildtype or the indicated variant seHAS, subjected to SDS-PAGE and the gels were either stained with Coomassie Brilliant Blue for quantification of HAS protein expression (B) or electroblotted to nitrocellulose for detection of HAS protein by Western analysis (A). Note that the relative migration (A) of seHAS(E327D) and seHAS(K48E) were both significantly slower than wildtype seHAS (indicated by the dashed line).

FIG. 17. SEC analysis of HA synthesized by membrane-bound wildtype seHAS or seHAS variants. Unlabeled HA was synthesized by membranes containing wildtype seHAS (WT, solid lines), or the indicated mutants and the samples were processed and analyzed by SEC-MALLS. The lines show refractive index values. (A) HA made by the E327D (dashed line) or E327Q (dotted line) seHAS mutants. (B) HA made by wildtype (WT, solid line) or the K48E (dotted line) or K48R (dashed line) seHAS mutants. (C) HA made by the double-switch seHAS(K48E,E327K) mutant (dashed line) is ˜one-sixth the size of WT HA.

FIG. 18. SEC-MALLS analysis of HA synthesized by membrane-bound wildtype seHAS or seHAS(K48E,E327K). Unlabeled HA was synthesized by membranes containing wildtype seHAS (solid line, filled circles) or the double-switch mutant (dashed line, filled triangles) and then processed and analyzed by SEC-MALLS. Light scattering data were used to determine the weight-average molar mass of the HA products (symbols). The lines show refractive index values.

FIG. 19. Gel filtration of HA product for xlHAS1 wild type and S77 mutants. The reactions were carried out using membranes as described, and analyzed with a PolySep-GFC-P 5000 (A and B) or 6000 (C-E) column. Incubation times were as follows: 2.5 min (A), 5 min (B), 15 min (C), 30 min (D), and 60 min (E). A, B, and D were stopped with 2% SDS. C and E were stopped with 0.5% SDS and treatment with Pronase followed by ultrafiltration. The data points are shown for xlHAS1 wild type (black dots; typical HA peak was 5,000-20,000 DPM [¹⁴C]GlcUA incorporation), whereas moving average trend lines represent the data for the mutants. The peaks of the 1300-, 600-, and 80-kDa MANT-labeled HA standards eluted at 11.7 (void volume), 11.7 (void volume), and 14.7 min, respectively, on the 5000 column and 13.2, 14.0, and 16.1 min, respectively, on the 6000 column.

FIG. 20. Gel filtration of HA product for xlHAS1 wild type and cysteine mutants. The reactions were carried out using membranes as described, and analyzed with a PolySep-GFC-P 5000 (A and B) or 6000 (C-E) column. Incubation times were as follows: 2.5 min (A), 5 min (B), 15 min (C), 30 min (D), and 60 min (E). A, B, and D were stopped with 2% SDS. C and E were stopped with 0.5% SDS and treatment with Pronase followed by ultrafiltration. The data points are shown for xlHAS1 wild type (black dots), whereas moving average trend lines represent the data for the mutants.

FIG. 21. Agarose gel analysis of HA product of xlHAS1 wild type and mutant enzymes. Duplicate reactions from C of FIGS. 19 and 20 (30 min) were also run on a 1.35% agarose gel and visualized as described. Lane 1, wild type xlHAS1; lane 2, Cl 17S; lane 3, C239S; lane 4, C337S; lane 5, S77F; lane 6, S77I; lane 7, S77T. The positions of the 1-kb DNA ladder standards are indicated with bars (corresponding to HA of 200, 250, 300, 400, 500, 700, and 900 kDa, from bottom to top, in size). The arrow marks the position of the sample well. Wild type xlHAS1 produced HA (WT marked with a bracket) with a size of 2-9×10⁵ Da in this experiment. The agarose gel data qualitatively corroborates the gel filtration analyses.

FIG. 22. Agarose Gel Analysis of HA produced by the seHAS ⁵¹LSF⁵³ Mutants. The enzymes indicated below were expressed in B. subtilis RB227 cells. The letters in brackets indicate the amino acids present in the ⁵¹LSF⁵³ region. After culture incubation and processing as described in the Methods, 4 μl of clarified media was loaded on a 0.6% agarose gel and electrophoresed for ˜6 hours with increasing voltage. HA was visualized by staining with Stains-All. Lanes: 1—MegaLadder; 2—HiLadder & LoLadder; 3—seHAS; 4—seHAS; 5—seHAS[FFF]; 6—seHAS[FFI]; 7—seHAS[FLT]; 8—seHAS[FTL]; 9—seHAS[FTT]; 10—seHAS[IIS]; 11—seHAS[ILF]; 12—seHAS[ISF]; 13—seHAS[ISI]; 14—seHAS[ISL]; 15—seHAS[ISS]; 16—seHAS[IST]; 17—seHAS[LIL]; 18—seHAS[LSS]; 19—seHAS[LST]; 20—seHAS[LTF]; 21—seHAS[LTT]; 22—seHAS[SFI]; 23—seHAS[STI]; 24—seHAS[TIS]; 25—seHAS[TIT]; 26—seHAS[TSI]; 27—seHAS[TSL]; 28—seHAS[TSS]; 29—seHAS[TST]; 30—seHAS[TSL].

FIG. 23. Agarose Gel Analysis of HA Produced by HAS Containing Multiple Mutation Sites. The enzymes indicated below were expressed in B. subtilis RB227 cells. After culture incubation and processing as described in the Methods, 9 μl of clarified media was loaded on a 0.6% agarose gel and electrophoresed for ˜6 hours with increasing voltage. HA was visualized by staining with Stains-All. A) Single mutants, B) Double mutants, C) Triple mutants, D) Quadruple mutants, E) Summary.

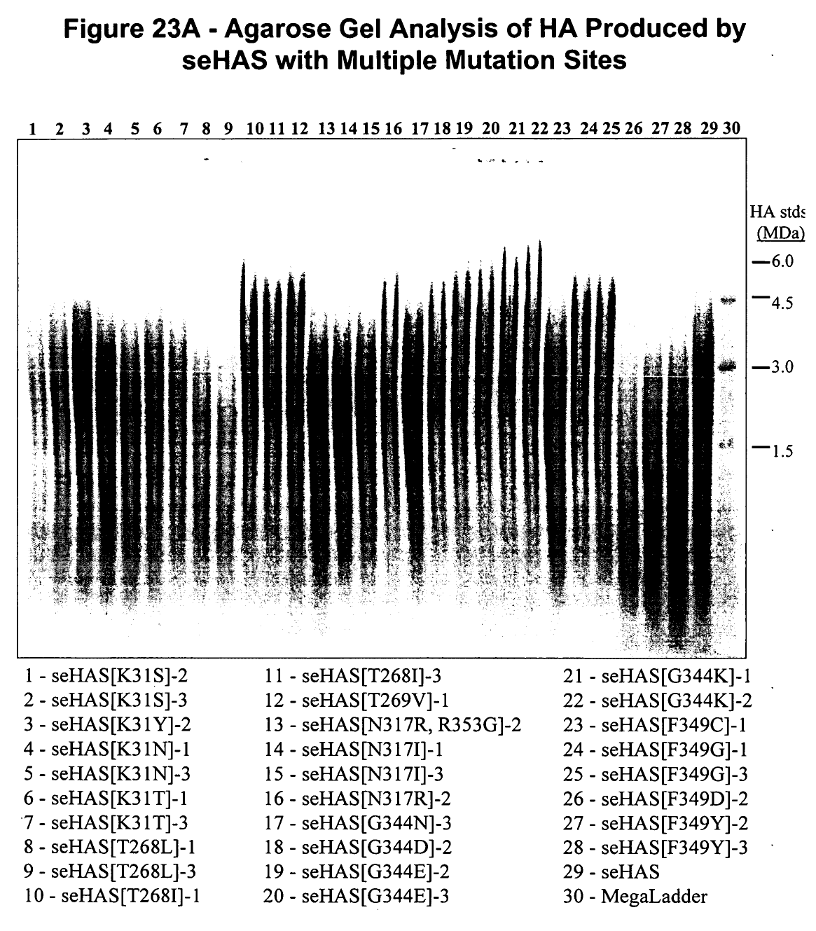

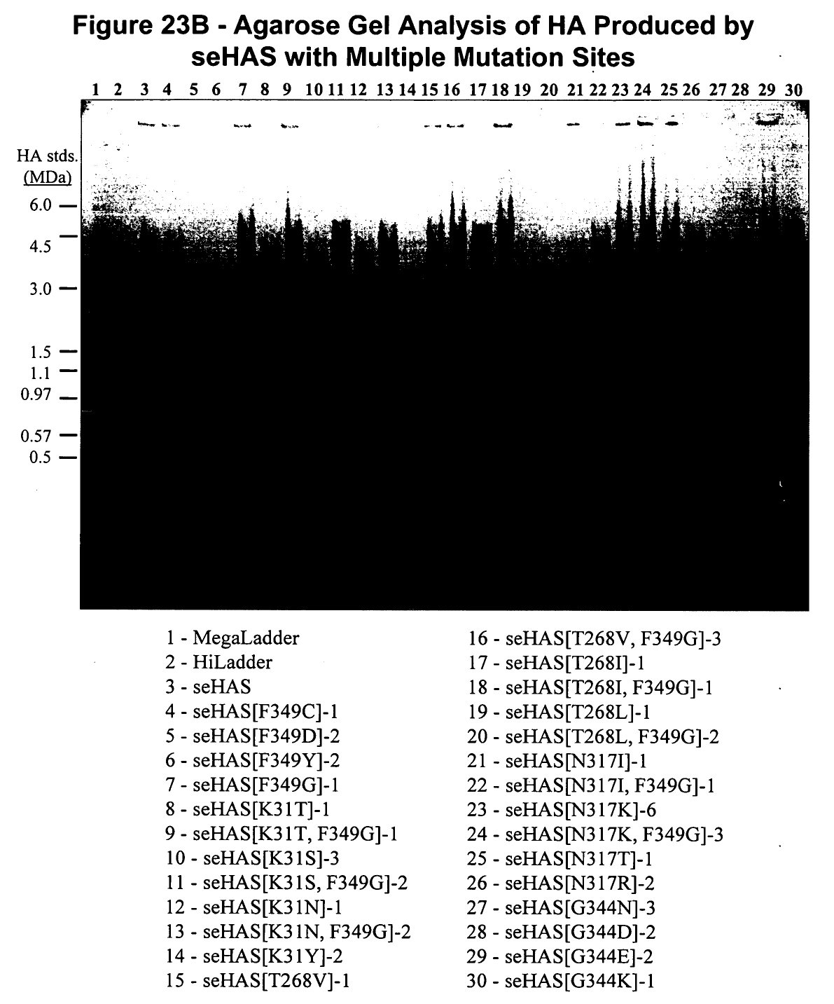

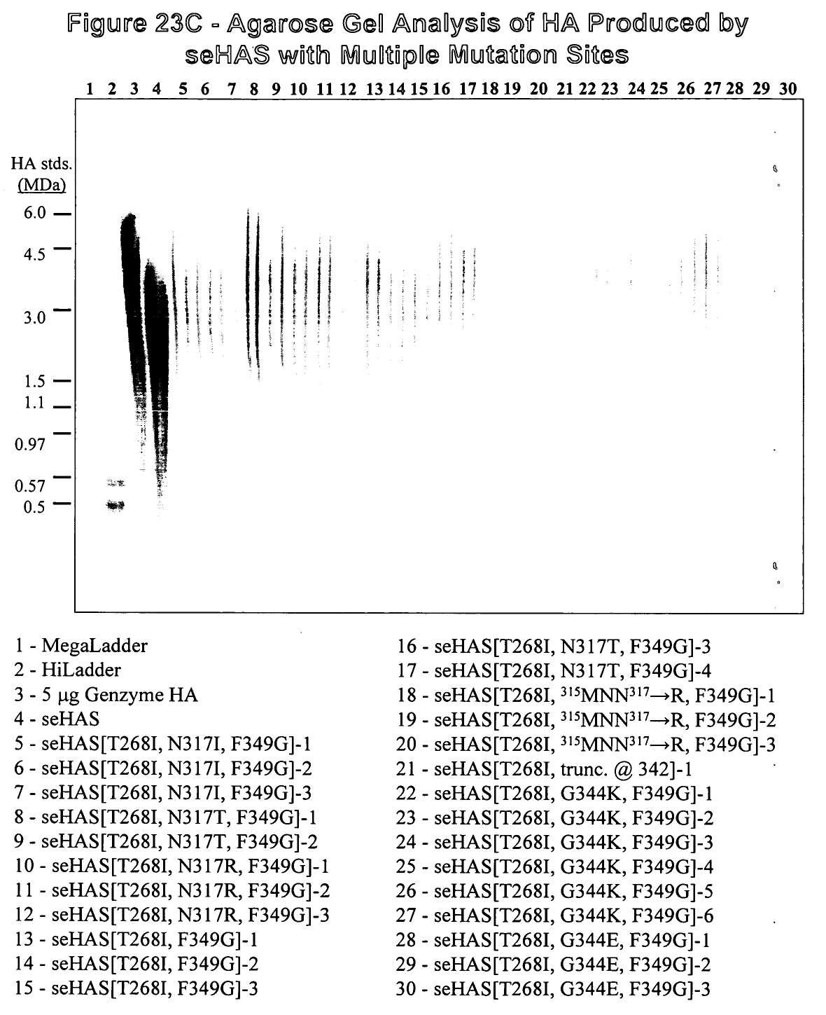

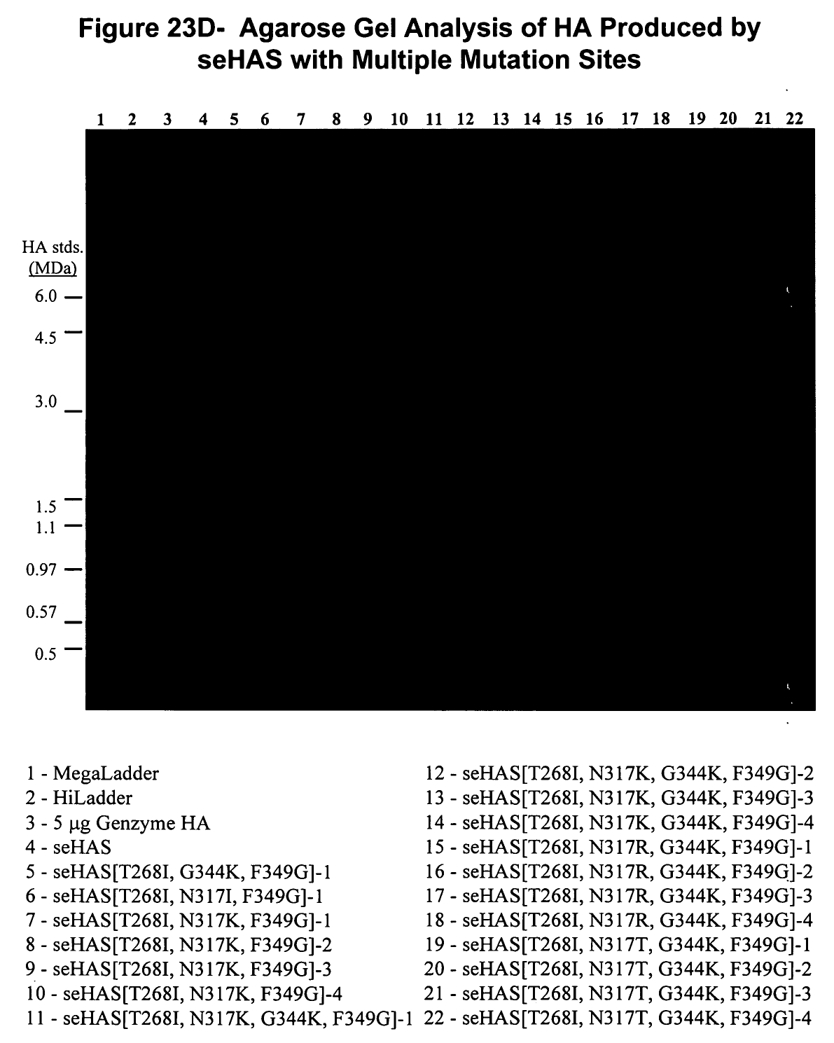

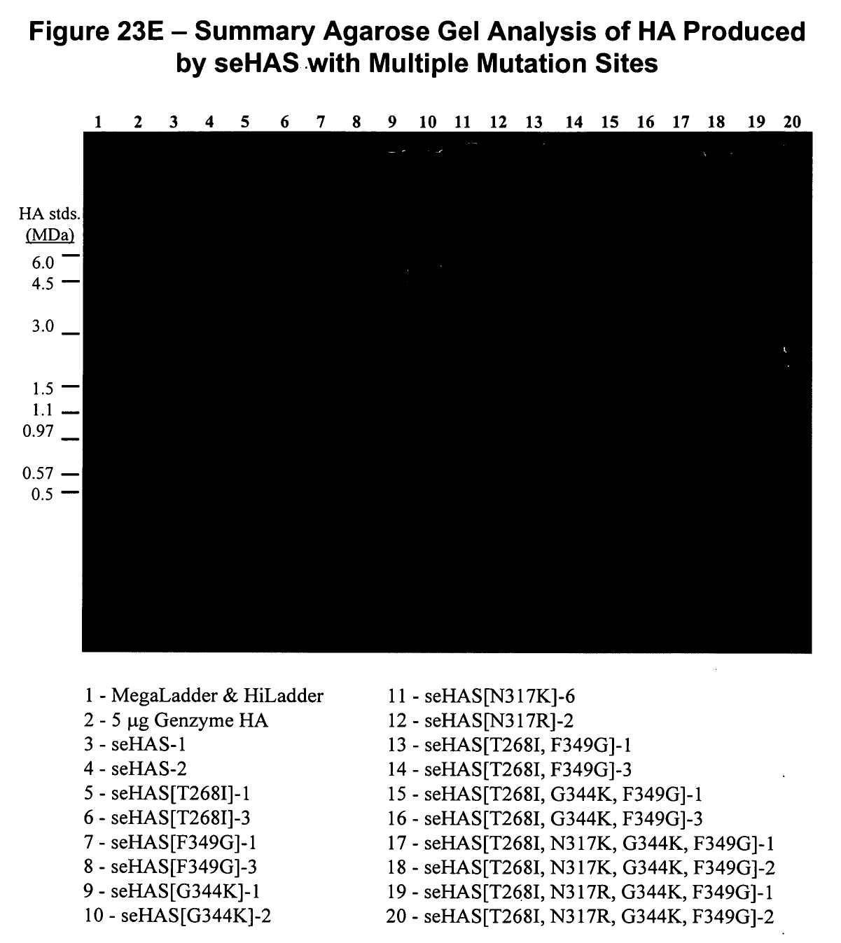

Lanes of FIG. 23A: 1—seHAS[K31S]-2; 2—seHAS[K31S]-3; 3—seHAS[K31Y]-2; 4—seHAS[K31N]-1; 5—seHAS[K31N]-3; 6—seHAS[K31T]-1; 7—seHAS[K31T]-3; 8—seHAS[T268L]-1; 9—seHAS[T268L]-3; 10—seHAS[T268I]-1; 11—seHAS[T268I]-3; 12—seHAS[T269V]-1; 13—seHAS[N317R, R353G]-2; 14—seHAS[N317I]-1; 15—seHAS[N317I]-3; 16—seHAS[N317R]-2; 1713 seHAS[G344N]-3; 18—seHAS[G344D]-2; 19—seHAS[G344E]-2; 20—seHAS[G344E]-3; 21—seHAS[G344K]-1; 22—seHAS[G344K]-2; 23—seHAS[F349C]-1; 24—seHAS[F349G]-1; 25—seHAS[F349G]-3; 26—seHAS[F349D]-2; 27—seHAS[F349Y]-2; 28—seHAS[F349Y]-3; 29—seHAS; 30—MegaLadder.

Lanes of FIG. 23B: 1—MegaLadder; 2—HiLadder; 3—seHAS; 4—seHAS[F349C]-1; 5—seHAS[F349D]-2; 6—seHAS[F349Y]-2; 7—seHAS[F349G]-1; 8—seHAS[K31T]-1; 9—seHAS[K31T, F349G]-1; 10—seHAS[K31S]-3; 11—seHAS[K31S, F349G]-2; 12—seHAS[K31N]-1; 13—seHAS[K31N, F349G]-2; 14—seHAS[K31Y]-2; 15—seHAS[T268V]-1; 16—seHAS[T268V, F349G]-3 17—seHAS[T268I]-1; 18—seHAS[T268I, F349G]-1; 19—seHAS[T268L]-1; 20—seHAS[T268L, F349G]-2; 21—seHAS[N317I]-1; 22—seHAS[N317I, F349G]-1; 23—seHAS[N317K]-6; 24—seHAS[N317K, F349G]-3; 25—seHAS[N317T]-1; 26—seHAS[N317R]-2; 27—seHAS[G344N]-3; 28—seHAS[G344D]-2; 29—seHAS[G344E]-2; 30—seHAS[G344K]-1.

Lanes of FIG. 23C: 1—MegaLadder; 2—HiLadder; 3-5 μg Genzyme HA; 4—seHAS; 5—seHAS[T268I, N317I, F349G]-1; 6—seHAS[T268I, N317I, F349G]-2; 7—seHAS[T268I, N317I, F349G]-3; 8—seHAS[T268I, N317T, F349G]-1; 9—seHAS[T268I, N317T, F349G]-2; 10—seHAS[T268I, N317R, F349G]-1; 11—seHAS[T268I, N317R, F349G]-2; 12—seHAS[T268I, N317R, F349G]-3; 13—seHAS[T268I, F349G]-1; 14—seHAS[T268I, F349G]-2; 15—seHAS[T268I, F349G]-3; 16—seHAS[T268I, N317T, F349G]-3; 17—seHAS[T268I, N317T, F349G]-4; 18—seHAS[T268I, ³¹⁵MNN³¹⁷→R, F349G]-1; 19—seHAS[T268I, ³¹⁵MNN³¹⁷→R, F349G]-2; 20—seHAS[T268I, ³¹⁵MNN³¹⁷→R, F349G]-3; 21—seHAS[T268I, trunc. @ 342]-1; 22—seHAS[268I, G344K, F349G]-1; 23—seHAS[T268I, G344K, F349G]-2; 24—seHAS[T268I, G344K, F349G]-3; 25—seHAS[T268I, G344K, F349G]-4; 26—seHAS[T268I, G344K, F349G]-5; 27—seHAS[T268I, G344K, F349G]-6; 28—seHAS[T268I, G344E, F349G]-1; 29—seHAS[T268I, G344E, F349G]-2; 30—seHAS[T268I, G344E, F349G]-3.

Lanes of FIG. 23D: 1—MegaLadder; 2—HiLadder; 3-5 μg Genzyme HA; 4—seHAS; 5—seHAS[T268I, G344K, F349G]-1; 6—seHAS[T268I, N317I, F349G]1; 7—seHAS[T268I, N317K, F349G]-1; 8—seHAS[T268I, N317K, F349G]-2; 9—seHAS[T268I, N317K, F349G]-3; 10—seHAS[T268I, N317K, F349G]4; 11—seHAS[T268I, N317K, G344K, F349G]-1; 12—seHAS[T268I, N317K, G344K, F349G]-2; 13—seHAS[T268I, N317K, G344K, F349G]-3; 14—seHAS[T268I, N317K, G344K, F349G]-4; 15—seHAS[T268I, N317R, G344K, F349G]-1; 16—seHAS[T268I, N317R, G344K, F349G]-2; 17—seHAS[T268I, N317R, G344K, F349G]-3; 18—seHAS[T268I, N317R, G344K, F349G]-4; 19—seHAS[T268I, N317T, G344K, F349G]-1; 20—seHAS[T268I, N317T, G344K, F349G]-2; 21—seHAS[T268I, N317T, G344K, F349G]-3; 22—seHAS[T268I, N317T, G344K, F349G]-4.

Lanes of FIG. 23E: 1—MegaLadder & HiLadder; 2-5 μg Genzyme HA; 3—seHAS-1; 4—seHAS-2; 5—seHAS[T268I]-1; 6—seHAS[T268I]-3; 7—seHAS[F349G]-1; 8—seHAS[F349G]-3; 9—seHAS[G344K]-1; 10—seHAS[G344K]-2; 11—seHAS[N317K]-6; 12—seHAS[N317R]-2; 13—seHAS[T268I, F349G]-1; 14—seHAS[T268I, F349G]-3; 15—seHAS[T268I, G344K, F349G]-1; 16—seHAS[T268I, G344K, F349G]-3; 17—seHAS[T268I, N317K, G344K, F349G]-1; 18—seHAS[T268I, N317K, G344K, F349G]-2; 19—seHAS[T268I, N317R, G344K, F349G]-1; 20—seHAS[T268I, N317R, G344K, F349G]-2.

### DETAILED DESCRIPTION OF THE INVENTION

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The present invention is directed to a functionally active hyaluronan synthase having at least one modified amino acid residue therein as compared to a corresponding functionally active native hyaluronan synthase. The term “modified amino acid residue” as used herein will be understood to include mutated amino acid residues as well as other modifications to amino acid residues, including but not limited to post-translational modifications of the amino acid residue, such as phosphorylations, glycosylations, methylations, prenylations, and the like. When the amino acid residue is modified by mutation, the mutation may arise by random mutagenesis ortargeted or “site directed” mutagenic techniques. For example, it may be desired to target a specific amino acid residue of the hyaluronan synthase to determine if a specific property of enzyme is affected. In one embodiment, it may be desired to target one or more Cysteines of the hyaluronan synthase to determine its involvement in disulfide bond formation or enzymatic activity of the hyaluronan synthase. In another embodiment, it may be desired to target one or more of a possible charge/polar-pair between two membrane domains to determine their involvement in anchoring. Alternatively, one may randomly mutagenize the gene encoding hyaluronan synthase and then screen or select for hyaluronan synthase mutants that produce altered amounts of HA as compared with the corresponding wild type HAS (that is, larger or smaller amounts of HA), or for hyaluronan synthase mutants that produce HA having an altered size as compared with HA produced by the corresponding wild type HAS (i.e., larger or smaller HA), without regard for the amino acid(s) that are mutated.

The terms “native” and “wild-type” are used herein interchangeably and will be understood to refer to a gene or allele that is the most frequently observed genotype and/or phenotype in nature and thus is arbitrarily designated as “normal”. Thus, a “native” or “wild-type” gene is that sequence normally found in an organism in the “wild” or “native” state.

In one embodiment of the present invention, the corresponding functionally active hyaluronan synthase is selected from the group consisting of spHAS, seHAS, suHAS, and pmHAS. The corresponding functionally active hyaluronan synthase may have an amino acid sequence essentially as set forth in at least one of SEQ ID NOS:2, 4, 6, 8, 10, 12 and 14.

In another embodiment of the present invention, at least one of the modified target amino acid residues is selected from the group consisting of an asparagine, a cysteine, a glycine, a lysine, a phenylalanine, a threonine, and combinations thereof. In a different embodiment, the functionally active hyaluronan synthase having at least one modified target amino acid residue therein has an amino acid sequence comprising at least one SEQ ID NOS: 15-160, such as but not limited to, SEQ ID NOS:15-92, SEQ ID NOS:93-98, SEQ ID NOS:99-123, or SEQ ID NOS:124-160.

Additionally, the present invention relates to a functionally active hyaluronan synthase having an altered enzymatic activity as compared to a corresponding functionally active native hyaluronan synthase. The term “altered enzymatic activity” as defined herein will be understood to refer to increased or decreased enzymatic activities, or activities that are enzymatically faster or slower than the native enzyme. In addition, the term “altered enzymatic activity” as used herein will also be understood to include enzymes that produce HA products having an altered size, that is, an HA polymer that has a an average molecular mass that is greater or less than the average molecular mass of an HA polymer produced by the native enzyme.

In a different embodiment of the present invention, the functionally active hyaluronan synthase having an altered enzymatic activity is selected from the group consisting of spHAS, seHAS, suHAS and pmHAS and has at least one modified target amino acid residue therein as compared to a corresponding functionally active native hyaluronan synthase. Further, in this embodiment of the invention, at least one of the modified target amino acid residues is selected from the group consisting of an asparagine, a cysteine, a glycine, a lysine, a phenylalanine, a threonine, and combinations thereof. In a different embodiment, the functionally active hyaluronan synthase having at least one modified target amino acid residue therein has an amino acid sequence comprising at least one SEQ ID NOS:15-160, such as but not limited to, SEQ ID NOS:15-92, SEQ ID NOS:93-98, SEQ ID NOS:99-123, or SEQ ID NOS:124-160.

Additionally, the present invention relates to a host cell having a functionally active hyaluronan synthase having an altered enzymatic activity as compared to a corresponding functionally active native hyaluronan synthase incorporated therein such that the host cell is capable of producing hyaluronan. In a separate embodiment of the present invention, the functionally active hyaluronan synthase having an altered enzyme activity is selected from the group consisting of spHAS, seHAS, suHAS, and pmHAS. Further, the functionally active hyaluronan synthase has at least one modified target amino acid therein as compared to a corresponding functionally active native hyaluronan synthase. The at least one of the modified target amino acid residues is selected from the group consisting of an asparagine, a cysteine, a glycine, a lysine, a phenylalanine, a threonine, and combinations thereof. In a different embodiment, the functionally active hyaluronan synthase having at least one modified target amino acid residue therein has an amino acid sequence comprising at least one SEQ ID NOs:15-160, such as but not limited to, SEQ ID NOS:15-92, SEQ ID NOS:93-98, SEQ ID NOS:99-123, or SEQ ID NOS:124-160.

Further, the present invention relates to a functionally active hyaluronan synthase having an amino acid sequence comprising SEQ ID NOS:15-160 such as but not limited to, SEQ ID NOS:5-92, SEQ ID NOS:93-98, SEQ ID NOS:99-123, or SEQ ID NOS:124-160. Also, the present invention relates to a functionally active hyaluronan synthase having an amino acid sequence as essentially set forth in SEQ ID NOS:15-160, such as but not limited to, SEQ ID NOS:15-92, SEQ ID NOS:93-98, SEQ ID NOS:99-123, or SEQ ID NOS:124-160.

Moreover, the present invention is directed to a method of providing a functionally active hyaluronan synthase having an altered enzymatic activity as compared to a corresponding functionally active native hyaluronan synthase. The method includes providing a hyaluronan synthase and modifying at least one target amino acid residue of the hyaluronan synthase to provide a functionally active hyaluronan synthase having an altered enzymatic activity.

In a separate embodiment of the present invention, the hyaluronan synthase is selected from the group consisting of spHAS, seHAS, suHAS, and pmHAS and the at least one modified target amino acid residue is selected from the group consisting of an asparagine, a cysteine, a glycine, a lysine, a phenylalanine, a threonine, and combinations thereof. The functionally active hyaluronan synthase having an altered enzymatic activity has an amino acid sequence essentially as set forth in at least one of SEQ ID NOS:15-160, such as but not limited to, SEQ ID NOS:15-92, SEQ ID NOS:93-98, SEQ ID NOS:99-123, or SEQ ID NOS:124-160.

In addition, the present invention is related to a method for producing hyaluronic acid. The method includes providing a host cell having at least one expression construct comprising a hyaluronan synthase gene encoding a functionally active hyaluronan synthase incorporated therein such that the host cell is capable of producing hyaluronan, wherein the functionally active hyaluronan synthase has an altered enzymatic activity as compared to a corresponding functionally active native hyaluronan synthase. The host cell is then cultured under conditions appropriate for the production of hyaluronic acid. The method may further include separating the hyaluronic acid from the host cell. The altered enzymatic activity of the functionally active hyaluronan synthase may be an increased or decreased enzymatic activity, or the hyaluronan synthase may produce hyaluronic acid having an average molecular mass that is greater than or less than an average molecular mass of hyaluronic acid produced by a corresponding functionally active native hyaluronan synthase.

The corresponding functionally active native hyaluronan synthase may have an amino acid sequence essentially as set forth in at least one of SEQ ID NOS:2,4, 6, 8, 10, 12 and 14, while the functionally active hyaluronan synthase having an altered enzymatic activity may have an amino acid sequence essentially as set forth in at least one of SEQ ID NOS:15-160, such as but not limited to, SEQ ID NOS:15-92, SEQ ID NOS:93-98, SEQ ID NOS:99-123, or SEQ ID NOS:124-160. The functionally active hyaluronan synthase having an altered enzymatic activity may have at least one modified amino acid residue therein as compared to the corresponding functionally active native hyaluronan synthase, and the at least one modified amino acid residue may be selected from the group consisting of an asparagine, a cysteine, a glycine, a lysine, a phenylalanine, a threonine, and combinations thereof.

In another embodiment of the method of producing hyaluronic acid, the expression construct may further include at least one gene encoding an enzyme for synthesis of a hyaluronic acid sugar precursor. Optionally, the gene encoding an enzyme for synthesis of a hyaluronic acid sugar precursor may be present on a separate expression construct or may be chromosomally integrated. The enzyme for synthesis of a hyaluronic acid sugar precursor is selected from the group consisting of a pyrophosphorylase, a transferase, a mutase, a dehydrogenase, an epimerase capable of increasing production of UDP-GlcNAc or UDP-GlcUA, and combinations thereof.

In another embodiment of the method of producing hyaluronic acid, at least one biosynthetic pathway gene of a hyaluronic acid sugar precursor or at least one gene encoding an enzyme for synthesis of a hyaluronic acid sugar precursor may be provided, either in the same or different expression construct or chomosomally integrated into the host cell. In a further alternative embodiment of the method of the present invention, the hyaluronic sugar precursors may be expressed in the host cell by endogenous genes of the host cell.

In another embodiment of the method of producing hyaluronic acid, nutrients utilized for a hyaluronic acid sugar precursor biosynthetic pathway or nutrients supplying the hyaluronic acid sugar precursor biosynthetic pathway are fed or supplied to the host cell.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley lnterscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients. All patents, patent applications, publications, and literature references cited in this specification are hereby incorporated herein by reference in their entirety.

As utilized in accordance with the present disclosure, the following terms, unless otherwise indicated, shall be understood to have the following meanings:

The terms “hyaluronic acid synthase”, “hyaluronate synthase”, “hyaluronan synthase” and “HA synthase”, are used interchangeably herein to describe an enzyme that polymerizes a glycosaminoglycan polysaccharide chain composed of alternating glucuronic acid and N-acetylglucosamine sugars, β1,3 and β1,4 linked. The term “seHAS”, for example, describes the HAS enzyme derived from Streptococcus equisimilis, wherein expression of the gene encoding the seHAS enzyme correlates with virulence of Streptococcal Group A and Group C strains by providing a means of escaping phagocytosis and immune surveillance.

As used herein, the term “nucleic acid segment” and “DNA segment” are used interchangeably and refer to a DNA molecule which has been isolated free of total genomic DNA of a particular species. Therefore, a “purified” DNA or nucleic acid segment as used herein, refers to a DNA segment which contains a Hyaluronate Synthase (“HAS”) coding sequence yet is isolated away from, or purified free from, unrelated genomic DNA of the source cell. Included within the term “DNA segment” are DNA segments and smaller fragments of such segments and also recombinant vectors, including, for example, plasmids, cosmids, phage, viruses, and the like.

Similarly, a DNA segment comprising an isolated or purified HAS gene refers to a DNA segment including HAS coding sequences isolated substantially away from other naturally occurring genes or protein encoding sequences. In this respect, the term “gene” is used for simplicity to refer to a functional protein, polypeptide or peptide encoding unit. As will be understood by those in the art, this functional term includes genomic sequences, cDNA sequences or combinations thereof. “Isolated substantially away from other coding sequences” means that the gene of interest, in this case HAS, forms the significant part of the coding region of the DNA segment, and that the DNA segment does not contain large portions of naturally-occurring coding DNA, such as large chromosomal fragments or other functional genes or DNA coding regions. Of course, this refers to the DNA segment as originally isolated, and does not exclude genes or coding regions later added to, or intentionally left in, the segment by the hand of man.

Due to certain advantages associated with the use of prokaryotic sources, one will likely realize the most advantages upon isolation of the HAS gene from prokaryotes. In particular, one may choose to utilize a Class I or Class II HAS, such as a Class I HAS from S. equisimilis or S. pyogenes, or a Class II HAS from P. multocida.

Streptococcus is subdivided taxonomically into Lancefield Groups based on different cell wall carbohydrate antigens. There are 18 distinct groups, but the most common pathogens are A, B, C and D. Historically, the most common pathogens are also often given specific species names, but the unified Lancefield testing method is recognized as being a clear method of typing and thus a useful classification scheme. Streptococcus species that may be utilized as the source of the HAS gene include Group A Streptococcus, such as S. pyogenes and S. haemolyticus, and Group C Streptococcus, such as S. equi, S. equisimilis, S. zooepidemicus, S. uberis and S. dysgalactiae.

One such advantage of isolating the HAS gene from prokaryotes is that, typically, eukaryotic enzymes may require significant post-translational modifications that can only be achieved in a eukaryotic host. This will tend to limit the applicability of any eukaryotic HA synthase gene that is obtained. Moreover, those of ordinary skill in the art will likely realize additional advantages in terms of time and ease of genetic manipulation where a prokaryotic enzyme gene is sought to be employed. These additional advantages include (a) the ease of isolation of a prokaryotic gene because of the relatively small size of the genome and, therefore, the reduced amount of screening of the corresponding genomic library, and (b) the ease of manipulation because the overall size of the coding region of a prokaryotic gene is significantly smaller due to the absence of introns. Furthermore, if the product of the HAS gene (i.e., the enzyme) requires posttranslational modifications, these would best be achieved in a similar prokaryotic cellular environment (host) from which the gene was derived.

In one embodiment, DNA sequences in accordance with the present invention will further include genetic control regions which allow the expression of the sequence in a selected recombinant host. Of course, the nature of the control region employed will generally vary depending on the particular use (e.g., cloning host) envisioned.

In particular embodiments, the invention cncoerns isolated DNA segments and recombinant vectors incorporating DNA sequences which encode a HAS gene, that includes within its amino acid sequence an amino acid sequence in accordance with at least one of SEQ ID NOS:2, 4, 6, 8, 10, 12 and 14-160. Moreover, in other particular embodiments, the invention concerns isolated DNA segments and recombinant vectors incorporating DNA sequences which encode a gene that includes within its amino acid sequence the amino acid sequence of a HAS gene or DNA, and in particular to a HAS gene or cDNA, corresponding to at least one of Streptococcus equisimilis HAS, Streptococcus pyogenes HAS, Streptococcus uberis HAS, Pasteurella multocida HAS, Xenopus laevis HAS, and Sulfolobus solfataricus HAS. For example, where the DNA segment or vector encodes a full length HAS protein, or is intended for use in expressing the HAS protein, sequences may be those which are essentially as set forth in at least one of SEQ ID NOS:2, 4, 6, 8, 10, 12 and 14-160.

Nucleic acid segments having HA synthase activity may be isolated by the methods described herein. The term “a sequence essentially as set forth in SEQ ID NO:X” or “a sequence as set forth in SEQ ID NO:X” means that the sequence substantially corresponds to a portion of SEQ ID NO:X and has relatively few amino acids which are not identical to, or a biologically functional equivalent of, the amino acids of SEQ ID NO:X. The term “biologically functional equivalent” is well understood in the art and is further defined in detail herein, as a gene having a sequence essentially as set forth in SEQ ID NO:X or comprising SEQ ID NO:X, and that is associated with the ability of prokaryotes or eukaryotes to produce HA or a hyaluronic acid coat.

For instance, the seHAS and spHAS coding sequences are approximately 70% identical and rich in the bases adenine (A) and thymine (T). SeHAS base content is A−26.71%, C−19.13%, G−20.81%, and T−33.33% (A/T=60%), whereas spHAS is A−31.34%, C−16.42%, G−16.34%, and T−35.8% (A/T=67%). Those of ordinary skill in the art would be surprised that the seHAS coding sequence does not hybridize with the spHAS gene and vice versa, despite their being 70% identical. This unexpected inability to cross-hybridize could be due to short interruptions of mismatched bases throughout the open reading frames. The longest stretch of identical nucleotides common to both the seHAS and the spHAS coding sequences is only 20 nucleotides. In addition, the very A-T rich sequences will form less stable hybridization complexes than G-C rich sequences. Another possible explanation could be that there are several stretches of As or Ts in both sequences that could hybridize in a misaligned and unstable manner. This would put the seHAS and spHAS gene sequences out of frame with respect to each other, thereby decreasing the probability of productive hybridization.

Because of this unique phenomena of two genes encoding proteins which are 70% identical not being capable of cross-hybridizing to one another, it is beneficial to think of the claimed nucleic acid segment in terms of its function; i.e., a nucleic acid segment which encodes enzymatically active hyaluronate synthase. One of ordinary skill in the art would appreciate that a nucleic acid segment encoding enzymatically active hyaluronate synthase may contain conserved or semi-conserved substitutions to the sequences set forth in SEQ ID NOS:2, 4, 6, 8, 10, 12 and 14-160, and yet still be within the scope of the invention.

In particular, the art is replete with examples of practitioners ability to make structural changes to a nucleic acid segment (i.e., encoding conserved or semi-conserved amino acid substitutions) and still preserve its enzymatic or functional activity. See for example: (1) Risler et al. “Amino Acid Substitutions in Structurally Related Proteins. A Pattern Recognition Approach.” J. Mol. Biol. 204:1019-1029 (1988); (2) Niefind et al. “Amino Acid Similarity Coefficients for Protein Modeling and Sequence Alignment Derived from Main-Chain Folding Anoles.” J. Mol. Biol. 219:481-497 (1991) [similarity parameters allow amino acid substitutions to be designed]; and (3) Overington et al. “Environment-Specific Amino Acid Substitution Tables: Tertiary Templates and Prediction of Protein Folds,” Protein Science 1:216-226 (1992) [“Analysis of the pattern of observed substitutions as a function of local environment shows that there are distinct patterns . . . ” Compatible changes can be made.], the contents of each being expressly incorporated herein by reference in their entirety.

These references and countless others indicate that one of ordinary skill in the art, given a nucleic acid sequence, could make substitutions and changes to the nucleic acid sequence without changing its functionality. Also, a substituted nucleic acid segment may be highly identical and retain its enzymatic activity with regard to its unadulterated parent, and yet still fail to hybridize thereto.

The invention discloses nucleic acid segments encoding enzymatically active hyaluronate synthases, such as seHAS, spHAS, suHAS, xlHAS and pmHAS. Although seHAS and spHAS are 70% identical and both encode enzymatically active hyaluronate synthase, they do not cross hybridize. Thus, one of ordinary skill in the art would appreciate that substitutions can be made to the HAS nucleic acid segments listed in SEQ ID NOS: 1, 3, 5, 7, 9, 11 and 13 without deviating outside the scope and claims of the present invention. Standardized and accepted functionally equivalent amino acid substitutions are presented in Table I.

TABLE I

| | Conservative and Semi- |
|---|---|
| Amino Acid Group | Conservative Substitutions |
| NonPolar R Groups | Alanine, Valine, Leucine, Isoleucine, |
| | Proline, Methionine, Phenylalanine, |
| | Tryptophan |
| Polar, but uncharged, R Groups | Glycine, Serine, Threonine, Cysteine, |
| | Asparagine, Glutamine |
| Negatively Charged R Groups | Aspartic Acid, Glutamic Acid |
| Positively Charged R Groups | Lysine, Arginine, Histidine |

Another embodiment of the present invention is a purified nucleic acid segment that encodes a protein in accordance with SEQ ID NOS:2, 4, 6, 8, 10, 12 and 14-160 further defined as a recombinant vector. As used herein, the term “recombinant vector” refers to a vector that has been modified to contain a nucleic acid segment that encodes an HAS protein, or fragment thereof. The recombinant vector may be further defined as an expression vector comprising a promoter operatively linked to said HAS encoding nucleic acid segment.

A further embodiment of the present invention is a host cell, made recombinant with a recombinant vector comprising an HAS gene. The recombinant host cell may be a prokaryotic cell. In another embodiment, the recombinant host cell is a eukaryotic cell. As used herein, the term “engineered” or “recombinant” cell is intended to refer to a cell into which a recombinant gene, such as a gene encoding HAS, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene, a copy of a genomic gene, or will include genes positioned adjacent to a promoter not naturally associated with the particular introduced gene.

The recombinant host cell may further contain at least one gene encoding an enzyme for synthesis of a HA sugar precursor. The HA sugar precursor may be selected from a pyrophosphorylase, a transferase, a mutase, a dehydrogenase, an epimerase capable of increasing production of UDP-GlcNAc or UDP-GlcUA, and combinations thereof. The recombinant host cell may further contain a biosynthetic pathway gene of a HA sugar precursor or an enzyme for synthesis of a HA sugar precursor. These one or more genes may be present on the same expression construct as the HAS gene or on separate expression construct. Optionally, these genes may be chromosomally integrated, as described in more detail hereinbelow.

Where one desires to use a host other than Streptococcus, as may be used to produce recombinant HA synthase, it may be advantageous to employ a prokaryotic system such as E. coli, Bacillus strains, Lactococcus sp., or even eukaryotic systems such as yeast or Chinese hamster ovary, African green monkey kidney cells, VERO cells, or the like. Of course, where this is undertaken it will generally be desirable to bring the HA synthase gene under the control of sequences which are functional in the selected alternative host. The appropriate DNA control sequences, as well as their construction and use, are generally well known in the art as discussed in more detail hereinbelow. For example, in one embodiment, the host cell may be a Bacillus cell, such as a Bacillus subtilis or Bacillus licheniformis cell, and the vector introduced therein contains a Bacillus-compatible promoter to which the has gene is operably linked.

In one embodiment, the host cell is a Bacillus cell, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus metaterium, Bacillus pumilus, Bacillus stearothermophilus, Bacillus subtilis and Bacillus thuringienisis.

In other embodiments, the HA synthase-encoding DNA segments further include DNA sequences, known in the art functionally as origins of replication or “replicons”, which allow replication of contiguous sequences by the particular host. Such origins allow the preparation of extrachromosomally localized and replicating chimeric segments or plasmids, to which HA synthase DNA sequences are ligated. In some instances, the employed origin is one capable of replication in bacterial hosts suitable for biotechnology applications. However, for more versatility of cloned DNA segments, it may be desirable to alternatively or even additionally employ origins recognized by other host systems whose use is contemplated (such as in a shuttle vector).

The isolation and use of other replication origins such as the SV40, polyoma or bovine papilloma virus origins, which may be employed for cloning or expression in a number of higher organisms, are well known to those of ordinary skill in the art. In certain embodiments, the invention may thus be defined in terms of a recombinant transformation vector which includes the HA synthase coding gene sequence together with an appropriate replication origin and under the control of selected control regions.

Thus, it will be appreciated by those of skill in the art that other means may be used to obtain the HAS gene or cDNA, in light of the present disclosure. For example, polymerase chain reaction or RT-PCR produced DNA fragments may be obtained which contain full complements of genes or cDNAs from a number of sources, including other strains of Streptococcus, or from eukaryotic sources, such as cDNA libraries. Virtually any molecular cloning approach may be employed for the generation of DNA fragments in accordance with the present invention. Thus, the only limitation generally on the particular method employed for DNA isolation is that the isolated nucleic acids should encode a biologically functional equivalent HA synthase.

Once the DNA has been isolated, it is ligated together with a selected vector. Virtually any cloning vector can be employed to realize advantages in accordance with the invention. Typical useful vectors include plasmids and phages for use in prokaryotic organisms and even viral vectors for use in eukaryotic organisms. Examples include pKK223-3, pSA3, recombinant lambda, SV40, polyoma, adenovirus, bovine papilloma virus and retroviruses. However, it is believed that particular advantages will ultimately be realized where vectors capable of replication in both Lactococcus or Bacillus strains and E. coli are employed.

Vectors such as these, exemplified by the pSA3 vector of Dao and Ferretti or the pAT19 vector of Trieu-Cuot, et al., allow one to perform clonal colony selection in an easily manipulatedhost such as E. coli, followed by subsequent transfer back into a food grade Lactococcus or Bacillus strain for production of HA. These are benign and well studied organisms used in the production of certain foods and biotechnology products. These are advantageous in that one can augment the Lactococcus or Bacillus strain's ability to synthesize HA through gene dosaging (i.e., providing extra copies of the HA synthase gene by amplification) and/or inclusion of additional genes to increase the availability of HA precursors. The inherent ability of a bacterium to synthesize HA can also be augmented through the formation of extra copies, or amplification, of the plasmid that carries the HA synthase gene. This amplification can account for up to a 10-fold increase in plasmid copy number and therefore the HA synthase gene copy number.

Another procedure that would further augment HA synthase gene copy number is the insertion of multiple copies of the gene into the plasmid. Another technique would include integrating the HAS gene and/or the sugar precursor gene(s) into chromosomal DNA. This extra amplification would be especially feasible, since the bacterial HA synthase gene size is small. In some scenarios, the chromosomal DNA-ligated vector is employed to transfect the host that is selected for clonal screening purposes such as E. coli, through the use of a vector that is capable of expressing the inserted DNA in the chosen host.

In another embodiment, the HA synthase gene is introduced into the host cell chromosome via homologous or heterologous recombination. The has gene may be more stable in this configuration, especially without drug selection. Various vectors may be employed to introduce the has gene into Bacillus, such as pTLH or pKSV7, or into yeast, such as Ylp2l 1, or into animal cells, such as pcDNA/FRT. The DNA is first introduced into the host cell by transformation, transduction or electroporation. The DNA segment with the has gene is then stably integrated into the host chromosome. For example, the spHAS gene was used to repair a mutant Streptococcus chromosome by transduction and integration; this operation resulted in HA production (DeAngelis et al, 1993a).

Where a eukaryotic source such as dermal or synovial fibroblasts or rooster comb cells is employed, one will desire to proceed initially by preparing a cDNA library. This is carried out first by isolation of mRNA from the above cells, followed by preparation of double stranded cDNA using an enzyme with reverse transcriptase activity and ligation with the selected vector. Numerous possibilities are available and known in the art for the preparation of the double stranded cDNA, and all such techniques are believed to be applicable. One technique involves reverse transcription. Once a population of double stranded cDNAs is obtained, a cDNA library is prepared in the selected host by accepted techniques, such as by ligation into the appropriate vector and amplification in the appropriate host. Due to the high number of clones that are obtained, and the relative ease of screening large numbers of clones by the techniques set forth herein, one may desire to employ phage expression vectors, such as λgt11, λgt12, λGem11, and/or λZAP for the cloning and expression screening of cDNA clones.

In certain other embodiments, the invention concerns isolated DNA segments and recombinant vectors that include within their sequence a nucleic acid sequence essentially as set forth at least one of in SEQ ID NOS:1, 3, 5, 7, 9, 11, 13 or a variant thereof wherein the variant encodes an amino acid sequence essentially as set forth in at least one of SEQ ID NOs:15-160. The term “essentially as set forth in SEQ ID NO: X” or “as set forth in SEQ ID NO: X”, for example, is used in the same sense as described hereinabove and means that the nucleic acid sequence substantially corresponds to a portion of SEQ ID NO: X, and has relatively few codons which are not identical, or functionally equivalent, to the codons of SEQ ID NO: X. The term “functionally equivalent codon” is used herein to refer to codons that encode the same amino acid, such as the six codons for arginine or serine, and also refers to codons that encode biologically equivalent amino acids as set forth in Table I.

It will also be understood that amino acid and nucleic acid sequences may include additional residues, such as additional N- or C-terminal amino acids or additional 5′ or 3′ nucleic acid sequences, and yet still be essentially as set forth in one of the sequences disclosed herein, so long as the sequence meets the criteria set forth above, including the maintenance of biological protein activity where protein expression and enzyme activity are concerned. The addition of terminal sequences particularly applies to nucleic acid sequences which may, for example, include various non-coding sequences flanking either of the 5′ or 3′ portions of the coding region or may include various internal sequences, which are known to occurwithin genes. In particular, the amino acid sequence of the has gene product in eukaryotes appears to be 40% larger than that found in prokaryotes, yet the prokaryotic and eukaryotic HASs display essentially identical enzymologic abilities to synthesize HA.

Allowing for the degeneracy of the genetic code as well as conserved and semi-conserved substitutions, sequences which have between about 40% and about 80%; or between about 80% and about 90%; or between about 90% and about 99%; of nucleotides which are identical to the nucleotides of SEQ ID NOS: 1, 3, 5, 7, 9, 11, 13 or a variant thereof wherein the variant encodes an amino acid sequence essentially as set forth in at least one of SEQ ID NOs:15-160 will be sequences which are “essentially as set forth in SEQ ID NO: X” or “comprising SEQ ID NO: X”. Sequences which are essentially the same as those set forth in SEQ ID NOS:1, 3, 5, 7, 9, 11, 13 or a variant thereof wherein the variant encodes an amino acid sequence essentially as set forth in at least one of SEQ ID NOS:15-160 may also be functionally defined as sequences which are capable of hybridizing to a nucleic acid segment containing the complement of SEQ ID NOS: 1, 3, 5, 7, 9, 11, 13 or a variant thereof wherein the variant encodes an amino acid sequence essentially as set forth in at least one of SEQ ID NOS:15-160 under “standard stringent hybridization conditions,” “moderately stringent hybridization conditions,” “less stringent hybridization conditions,” or “low stringency hybridization conditions.” Suitable “standard” or “less stringent” hybridization conditions will be well known to those of skill in the art and are clearly set forth hereinbelow. In one embodiment, standard stringent hybridization conditions or less stringent hybridization conditions are utilized.

The terms “standard stringent hybridization conditions,” “moderately stringent conditions,” and “less stringent hybridization conditions” or “low stringency hybridization conditions” are used herein, describe those conditions under which substantially complementary nucleic acid segments will form standard Watson-Crick base-pairing and thus “hybridize” to one another. A number of factors are known that determine the specificity of binding or hybridization, such as pH; temperature; salt concentration; the presence of agents, such as formamide and dimethyl sulfoxide; the length of the segments that are hybridizing; and the like. There are various protocols for standard hybridization experiments. Depending on the relative similarity of the target DNA and the probe or query DNA, then the hybridization is performed under stringent, moderate, or under low or less stringent conditions.

The hybridizing portion of the hybridizing nucleic acids is typically at least about 14 nucleotides in length, such as between about 14 and about 100 nucleotides in length. The hybridizing portion of the hybridizing nucleic acid is at least about 60%, e.g., at least about 80% or at least about 90%, identical to a portion or all of a nucleic acid sequence encoding a HAS or its complement, such as SEQ ID NO: 2 or 4 or the complement thereof. Hybridization of the oligonucleotide probe to a nucleic acid sample typically is performed under standard or stringent hybridization conditions. Nucleic acid duplex or hybrid stability is expressed as the melting temperature or Tₘ, which is the temperature at which a probe nucleic acid sequence dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC, SSPE, or HPB). Then, assuming that 1% mismatching results in a 1 C decrease in the Tₘ, the temperature of the final wash in the hybridization reaction is reduced accordingly (for example, if sequences having >95% identity with the probe are sought, the final wash temperature is decreased by about 5 C.). In practice, the change in Tₘ can be between about 0.5° C. and about 1.5° C. per 1% mismatch. Examples of standard stringent hybridization conditions include hybridizing at about 68° C. in 5×SSC/5×Denhardt's solution/1.0% SDS, followed with washing in 0.2×SSC/0.1% SDS at room temperature or hybridizing in 1.8×HPB at about 30° C. to about 45° C. followed by washing a 0.2-0.5×HPB at about 45° C. Moderately stringent conditions include hybridizing as described above in 5×SSC\5×Denhardt's solution 1% SDS washing in 3×SSC at 42° C. The parameters of salt concentration and temperature can be varied to achieve the optimal level of identity between the probe and the target nucleic acid. Additional guidance regarding such conditions is readily available in the art, for example, by Sambrook et al., 1989, Molecular Cloning, A Laboratory Manual, (Cold Spring Harbor Press, N.Y.); and Ausubel et al. (eds.), 1995, Current Protocols in Molecular Biology, (John Wiley & Sons, N.Y.). Several examples of low stringency protocols include: (A) hybridizing in 5×SSC, 5×Denhardts reagent, 30% formamide at about 30° C. for about 20 hours followed by washing twice in 2×SSC,0.1% SDS at about 30° C. for about 15 min followed by 0.5×SSC, 0.1% SDS at about 30° C. for about 30 min (FEMS Microbiology Letters, 2000, vol.193, p. 99-103); (B) hybridizing in 5×SSC at about 45° C. ovemight followed by washing with 2×SSC, then by 0.7×SSC at about 55° C. (J. Biological Methods, 1990, vol. 30, p. 141-150); or (C) hybridizing in 1.8×HPB at about 30° C. to about 45° C.; followed by washing in 1×HPB at 23° C.

Naturally, the present invention also encompasses DNA segments which are complementary, or essentially complementary, to the sequence set forth in SEQ ID NOS:1, 3, 5, 7, 9, 11, 13 or a variant thereof wherein the variant encodes an amino acid sequence essentially as set forth in at least one of SEQ ID NOS:15-160. Nucleic acid sequences which are “complementary” are those which are capable of base-pairing according to the standard Watson-Crick complementarity rules. As used herein, the term “complementary sequences” means nucleic acid sequences which are substantially complementary, as may be assessed by the same nucleotide comparison set forth above, or as defined as being capable of hybridizing to the nucleic acid segment of SEQ ID NOS: 1, 3, 5, 7, 9, 11, 13 or a variant thereof wherein the variant encodes an amino acid sequence essentially as set forth in at least one of SEQ ID NOS:15-160.

The nucleic acid segments of the present invention, regardless of the length of the coding sequence itself, may be combined with other DNA sequences, such as promoters, polyadenylation signals, additional restriction enzyme sites, multiple cloning sites, epitope tags, poly histidine regions, other coding segments, and the like, such that their overall length may vary considerably. It is therefore contemplated that a nucleic acid fragment of almost any length may be employed, with the total length possibly being limited by the ease of preparation and use in the intended recombinant DNA protocol.

Naturally, it will also be understood that this invention is not limited to the particular nucleic acid and amino acid sequences of SEQ ID NOS:1, 3, 5, 7, 9, 11 or 13 (or a variant thereof wherein the variant encodes an amino acid sequence essentially as set forth in at least one of SEQ ID NOS:15-160) and SEQ ID NOS: 15-160, respectively. Recombinant vectors and isolated DNA segments may therefore variously include the HAS coding regions themselves, coding regions bearing selected alterations or modifications in the basic coding region, or they may encode larger polypeptides which nevertheless include HAS-coding regions or may encode biologically functional equivalent proteins or peptides which have variant amino acids sequences.

The terms “SEC-MALLS” and “GPC-MALLS” are used herein interchangeably and will be understood to refer to size partitioning chromatography. “SEC-MALLS” refers to size exclusion chromatography, while “GPC-MALLS” refers to gel permeation or gel filtration chromatography.

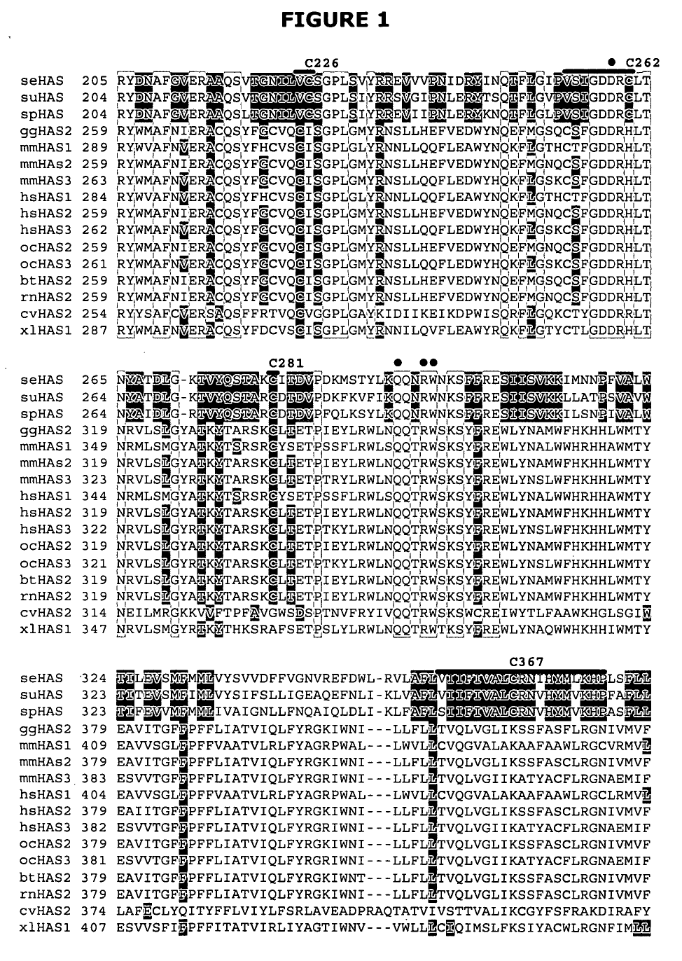

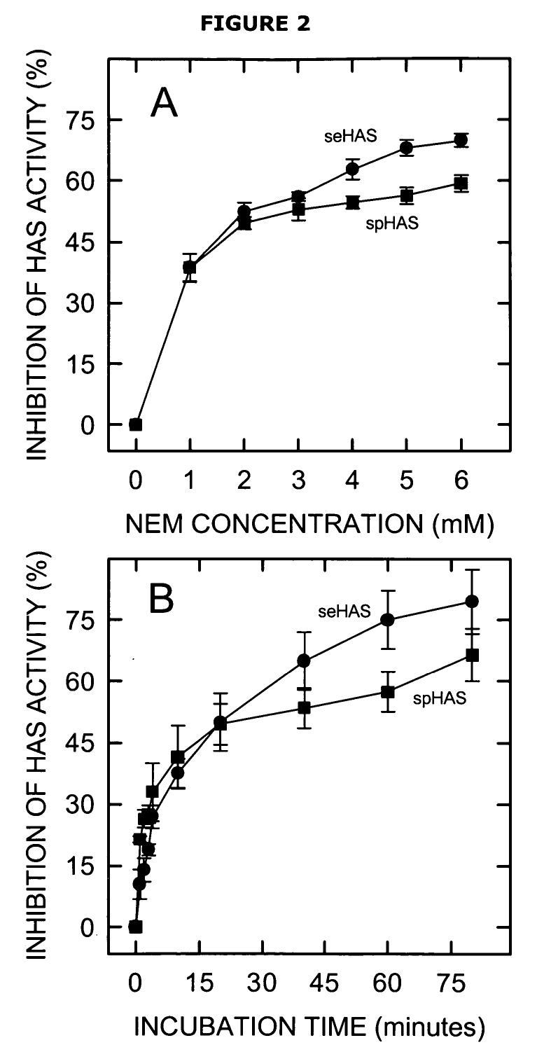

Sulfhydryl reagents inhibit the activity of seHAS and spHAS. SeHAS is the smallest HAS protein (417 amino acids) and contains four Cys residues. The four cysteines of seHAS are completely conserved among the three prokaryotic HASs (excluding pmHAS) and are conserved positionally among all the vertebrate HASs (FIG. 1). To explore the possible role of cysteines in the function of HAS, the activities of seHAS and spHAS were assayed in the presence of different sulfhydryl reagents (Table II). Almost identical sensitivities were observed for the two enzymes. For example, seHAS activity was inhibited >93% by methylmethanethiosulfonate (0.05 mM) and ˜70% by NEM (5 mM), whereas IAA inhibited only 15%. Sodium arsenite and 5,5′-dithiobis-(2-nitrobenzoic acid) also inhibited each HAS activity. These results indicate that one or more Cys residues are important for the overall HA synthesis activity of the seHAS and spHAS proteins. The inhibition of each HAS by NEM was examined in more detail with respect to time of incubation and NEM concentration (FIG. 2). Both seHAS and spHAS were inhibited in a biphasic manner, with respect to incubation time or NEM concentration. Although the extent of inhibition varied from experiment to experiment, a 60-70% effect was typical. About half of the observed inactivation occurred at ≦1 mM NEM, whereas the remaining inactivation occurred from 1-6 mM (FIG. 2A). Kinetically, there was a fast initial inactivation and then a much slower phase of inhibition; again each of the phases involved about half of the affected activity (FIG. 2B).

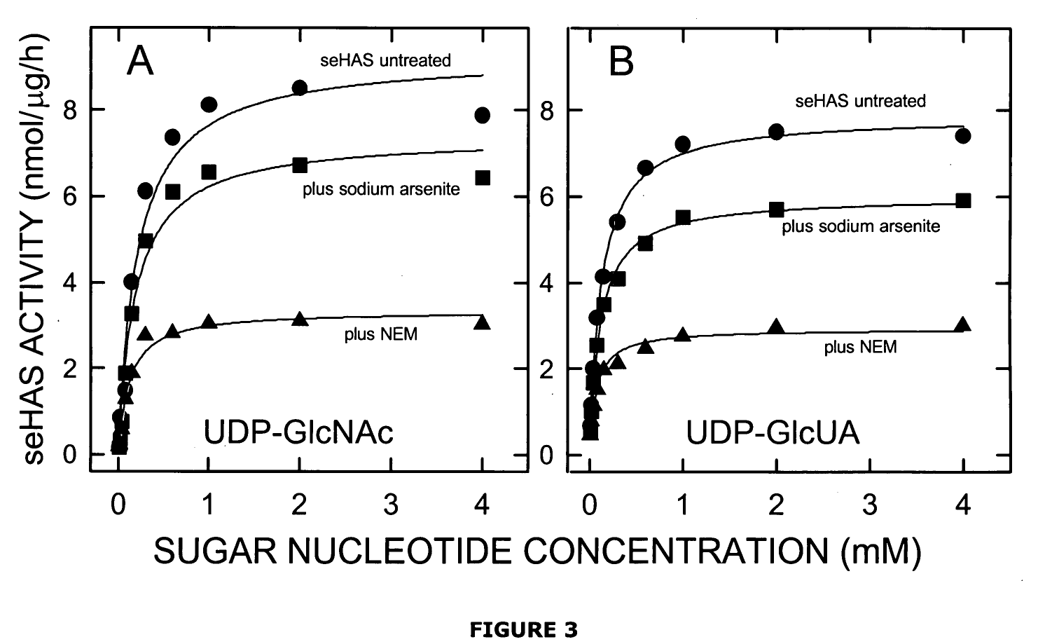

A potential complication in the above NEM studies is that the effects of a sulfhydryl modifying reagent may be due to secondary effects caused by modification of other molecules in the membranes being tested. Although this possibility is highly unlikely, since seHAS is the only protein necessary for HA biosynthesis, the effect of sulfhydryl reagents on the seHAS Cys-null mutant in isolated membranes was also examined under the conditions shown in FIG. 3.

The activity of seHAS^{Cys-null} was not affected (≦1%) by treatment with NEM, IAA or sodium arsenite, which eliminates the possibility that modified secondary proteins in the membranes preparations were responsible for the altered HAS activity.

TABLE II
Effect of different sulfhydryl reagents on seHAS or spHAS activity.
E. coli membranes expressing the recombinant seHAS-H₆ or spHAS-H₆
proteins were incubated at 4° C. for 1 h with PBS containing either
5 mM NEM, 5 mM IAA, 0.5 mM 5, 5′-dithiobis-(2-nitrobenzoic acid),
0.05 mM methylmethanethiosulfonate, 10 mM sodium arsenite or no
addition (control, which was set as 100%). The remaining seHAS
activity was then determined in quadruplicate and expressed as
% relative to the control. The mean values and standard deviations
are shown.

Inhibition of
HAS Activity
(% relative to control)

| Sulfhydryl Reagent | seHAS | spHAS |
|---|---|---|
| N-ethylmaleimide | 70 ± 4.8 | 60 ± 3.5 |
| Iodoacetic acid | 15 ± 6.0 | 13 ± 5.4 |
| 5, 5′-dithiobis-(2-nitrobenzoic acid) | 52 ± 6.5 | 52 ± 4.9 |
| Methylmethanethiosulfonate | 93 ± 5.4 | 89 ± 5.5 |
| Sodium Arsenite | 40 ± 4.0 | 46 ± 5.1 |

TABLE III
Effect of NEM or sodium arsenite treatment on the utilization
of UDP-GlcUA and UDP-GlcNAc by wild-type seHAS
E. coli membranes containing seHAS protein were incubated
at 4° C. for 1 h with PBS alone (control) or PBS containing
5 mM NEM or 10 mM sodium arsenite. The activity of seHAS was
determined in triplicate with varying concentrations of UDP-GlcUA or
UDP-GlcNAc as described in Materials and Methods, and the
Michaelis-Menten Constants (Kₘ and Vₘₐₓ) ± standard
errors were calculated.

| | UDP-GlcUA | | | UDP-GlcNAc | | |
|---|---|---|---|---|---|---|
| | | | Sodium | | | Sodium |
| | Control | NEM | Arsenite | Control | NEM | Arsenite |
| Kₘ | 85 ± 10 | 57 ± 5 | 67 ± 23 | 122 ± 30 | 82 ± 17 | 112 ± 38 |
| (μM) | | | | | | |
| Vₘₐₓ | 14.9 ± 3.6 | 7.3 ± 2.2 | 9.5 ± 1.1 | 14.9 ± 3.6 | 7.3 ± 2.2 | 9.5 ± 1.1 |
| (nmol/μg/h) | | | | | | |

Modification of the protein by NEM could affect any one or several of the six discrete functions that HAS must perform in order to synthesize HA. In order to determine if one of the nucleotide-sugar binding sites was affected by NEM, we examined the UDP-GlcUA and UDP-GlcNAc saturation profiles for treated and untreated seHAS (FIG. 3 and Table ll). The Kₘ values for either UDP-GlcNAc (FIG. 3A) or UDP-GlcUA (FIG. 3B) were not altered significantly by treatment with NEM or sodium arsenite, whereas the maximum enzymatic velocity was reduced by up to ˜70%.

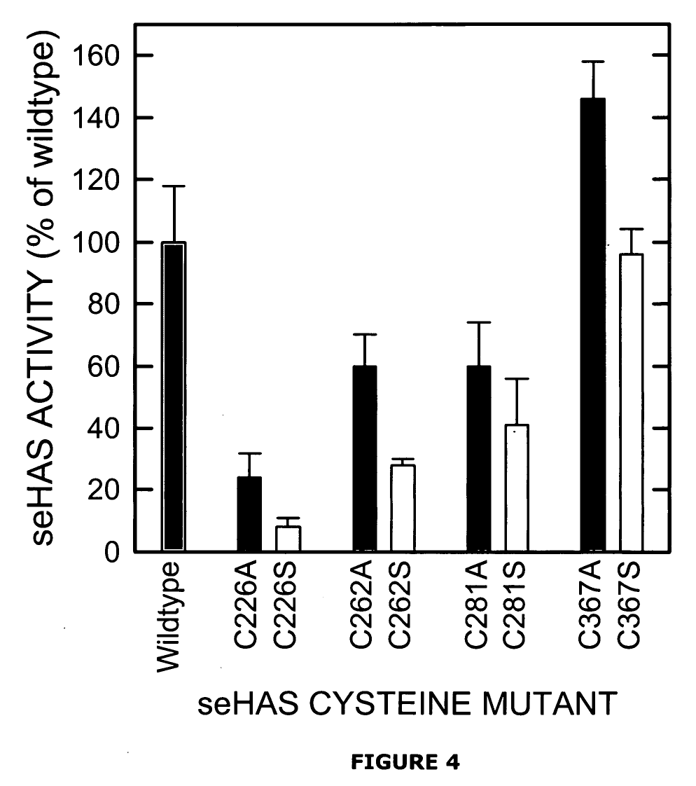

Effect of site-specific Cys mutagenesis on the HA synthase activity of seHAS. Site-specific Cys-to-Ala and Cys-to-Ser mutants of seHAS were made in order to explore the possible functional role of each Cys residue in HAS activity (see Table IV for a list of the seHAS Cys mutants). In all of the following kinetic studies using the wild-type (or native) and mutant seHAS proteins, the data obtained were normalized to the amount of intact seHAS protein as described herein. The single Cys-to-Ala or Cys-to-Ser mutants of seHAS had lower enzyme activities compared to the wild-type enzyme, except for the C367A and C367S variants (FIG. 4 and Table V). This result indicates that CyS²²⁶, CyS²⁶² and CyS²⁸¹ contribute to the catalytic activity of seHAS. The K^{m} values for UDP-GlcUA of the C226A and C262A mutants were higher when compared to the corresponding values for the C226S and C262S variants. However, the Kₘ values for UDP-GlcNAc were not quite as clear-cut. The K_{UDP-GlcNAc} value for the C226S mutant was higher than that of the C226A mutant, whereas the C262A and C281A mutant proteins both had higher K_{UDP-GlcNAc} values compared to the C262S and C281S variants. At the C367 position, similar Kₘ values for each nucleotide-sugar were obtained for both the Ala and Ser mutants.

TABLE IV
All Possible Cysteine Mutants of seHAS and spHAS.
Each of the Cysteines listed may be mutated to Alanine,
Serine, or any other amino acid as described herein previously.
In addition, when more than one Cysteine is mutated, all the
mutations may be the same (i.e., all Cys-Ala mutations), or a
mutant containing multiple Cysteine mutations may have a
combination of Cys-Ala, Cys-Ser and Cys-Xaa mutations.

| HAS | Cysteine(s) mutated | SEQ ID NO: |
|---|---|---|
| seHAS | 226 | 15 |
| seHAS | 262 | 16 |
| seHAS | 281 | 17 |
| seHAS | 367 | 18 |
| seHAS | 226, 262 | 19 |
| seHAS | 226, 281 | 20 |
| seHAS | 226, 367 | 21 |
| seHAS | 262, 281 | 22 |
| seHAS | 262, 367 | 23 |
| seHAS | 281, 367 | 24 |
| seHAS | 262, 281, 367 | 25 |
| seHAS | 226, 281, 367 | 26 |
| seHAS | 226, 262, 367 | 27 |
| seHAS | 226, 262, 281 | 28 |
| seHAS | Cys-null (226, 262, 281, 367) | 29 |
| spHAS | 124 | 30 |
| spHAS | 225 | 31 |
| spHAS | 261 | 32 |
| spHAS | 280 | 33 |
| spHAS | 366 | 34 |
| spHAS | 402 | 35 |
| spHAS | 125, 225 | 36 |
| spHAS | 124, 261 | 37 |
| spHAS | 124, 280 | 38 |
| spHAS | 124, 366 | 39 |
| spHAS | 124, 402 | 40 |
| spHAS | 225, 261 | 41 |
| spHAS | 225, 280 | 42 |
| spHAS | 225, 366 | 43 |
| spHAS | 225, 402 | 44 |
| spHAS | 261, 280 | 45 |
| spHAS | 261, 366 | 46 |
| spHAS | 261, 402 | 47 |
| spHAS | 280, 366 | 48 |
| spHAS | 280, 402 | 49 |
| spHAS | 366, 402 | 50 |
| spHAS | 124, 225, 261 | 51 |
| spHAS | 124, 225, 280 | 52 |
| spHAS | 124, 225, 366 | 53 |
| spHAS | 124, 225, 402 | 54 |
| spHAS | 124, 261, 280 | 55 |
| spHAS | 124, 261, 366 | 56 |
| spHAS | 124, 261, 402 | 57 |
| spHAS | 124, 280, 366 | 58 |
| spHAS | 124, 280, 402 | 59 |
| spHAS | 124, 366, 402 | 60 |
| spHAS | 225, 261, 280 | 61 |
| spHAS | 225, 261, 366 | 62 |
| spHAS | 225, 261, 402 | 63 |
| spHAS | 225, 280, 366 | 64 |
| spHAS | 225, 280, 402 | 65 |
| spHAS | 225, 366, 402 | 66 |
| spHAS | 261, 280, 366 | 67 |
| spHAS | 261, 280, 402 | 68 |
| spHAS | 261, 366, 402 | 69 |
| spHAS | 280, 366, 402 | 70 |
| spHAS | 124, 225, 261, 280 | 71 |
| spHAS | 124, 225, 261, 366 | 72 |
| spHAS | 124, 225, 261, 402 | 73 |
| spHAS | 124, 225, 280, 366 | 74 |
| spHAS | 124, 225, 280, 402 | 75 |
| spHAS | 124, 225, 366, 402 | 76 |
| spHAS | 124, 261, 280, 366 | 77 |
| spHAS | 124, 261, 280, 402 | 78 |
| spHAS | 124, 261, 366, 402 | 79 |
| spHAS | 124, 280, 366, 402 | 80 |
| spHAS | 225, 261, 280, 366 | 81 |
| spHAS | 225, 261, 280, 402 | 82 |
| spHAS | 225, 261, 366, 402 | 83 |
| spHAS | 225, 280, 366, 402 | 84 |
| spHAS | 261, 280, 366, 402 | 85 |
| spHAS | 124, 225, 261, 280, 366 | 86 |
| spHAS | 124, 225, 261, 280, 402 | 87 |
| spHAS | 124, 225, 261, 366, 402 | 88 |
| spHAS | 124, 225, 280, 366, 402 | 89 |
| spHAS | 124, 261, 280, 366, 402 | 90 |
| spHAS | 225, 261, 280, 366, 402 | 91 |
| spHAS | Cys-null (124, 225, 261, 280, 366, | 92 |
| | 402) | |

From these results it is shown that functional constraints are put on the HAS enzyme by particular alterations of at least one of its Cys residues. Since the C226A and C226S mutants were the least active, Cys²²⁶ appears to be the most important Cys residue for enzyme activity. The seHAS(C367A) variant was actually more active than wild-type (˜145%) and the seHAS(C367S) variant was not significantly altered. In each of the four cases, the Cys-to-Ala change resulted in a variant with greater activity than the Cys-to-Ser change. The least tolerated single Cys change was C226S; this mutant was inhibited >90%.

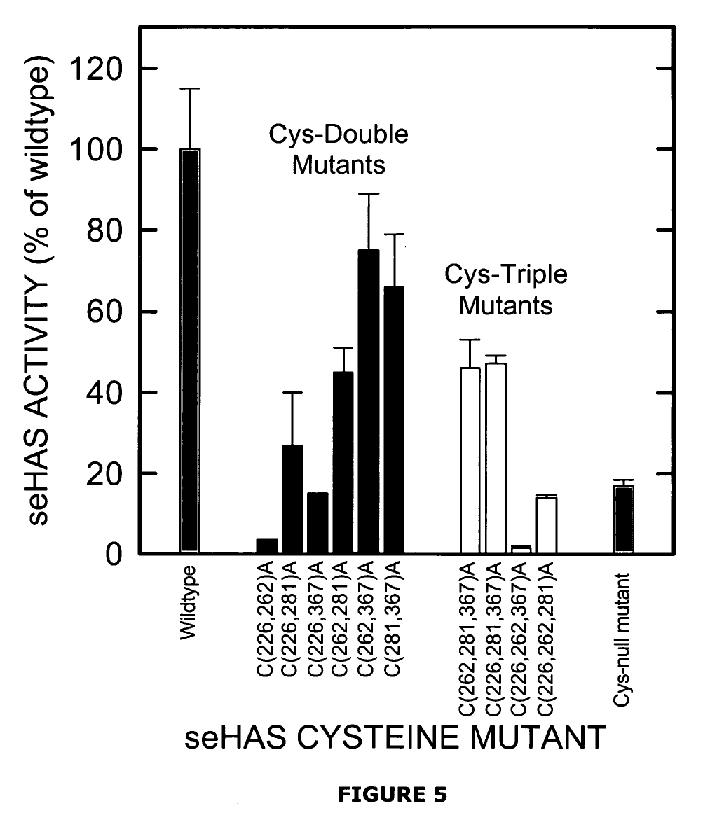

All the possible Cys-to-Ala double mutants (C226,262A; C226,281A; C226,367A; C262,281A; C262,367A; C281,367A) as well as the triple mutants and the Cys-null mutant were constructed and examined. For simplicity, the triple Cys-mutants are designated by a convention that indicates which of the four Cys residues remains unaltered. For example, the triple mutant containing C(226,281,367)A changes is seHAS(A3C)C²⁶², which has only one Cys at position 262 as in the wild-type protein. The HA synthase activities of these multiple-Cys seHAS mutants were then determined under saturating conditions for each substrate and normalized to the amount of seHAS protein present in the isolated membranes (FIG. 5). The least active double mutant was C(226,262)A, which had only 2-3% of the specific activity of the wild-type enzyme. All three double mutants, in which Cys²²⁶ was changed to Ala, had lower activity compared to the other three double mutants. Two of the triple mutants, seHAS(Δ3C)C²²⁶ and seHAS(A3C)C²⁶² were significantly more active (˜3-30 fold) than the other two triple mutants seHAS(A3C)C²⁸¹ and seHAS(A3C)C²⁸⁷.

Surprisingly, the Cys-null seHAS mutant was more active than the two least active triple Cys-mutants and two of the six double Cys-mutants (FIG. 5). The decreased activities of the single and multiple Cys-mutants are consistent with the inhibition of seHAS or spHAS by sulfhydryl reagents described above. Based on the lower specific activities of most of these Cys-mutants, it was concluded that no particular cysteine residue in seHAS is required for a critical step during HA synthesis. Nonetheless, these data also support the conclusion that Cys²²⁶ and Cys²⁶² may play a role, or at least influence, one or more of the six sub-activities required for the overall activity of HAS. At least the alteration or modification of these latter two residues hinders the enzyme and results in apparently lower Vₘₐₓ values.

TABLE V
Michaelis-Menton Constants for single Cys-Mutants of seHAS
Kinetic analyses were performed as described herein using
membranes prepared from E. coli SURE cells expressing
the indicated seHAS variants. Hill numbers for the wild-type seHAS
and single Cys-mutants of seHAS ranged from 0.9 to 1.2 and none
of the mutant values were significantly different from wild-type

| | | Km | |
|---|---|---|---|
| seHAS | Vmax | for UDPGIcUA | Km for UDPGIcNAc |
| MUTANTS | (nmol/pmol/h)^{a} | (μM) | (μM) |
| WT | 5.60 ± 0.48 | 77 ± 5 | 74 ± 7 |
| C226A | 1.34 ± 0.21 | 88 ± 17 | 154 ± 23^{b} |
| C226S | 0.45 ± 0.08^{c} | 44 ± 7^{b} | 232 ± 0.2^{c} |
| C262A | 3.36 ± 0.26 | 146 ± 41 | 186 ± 26^{b} |
| C262S | 1.56 ± 0.05 | 96 ± 10 | 153 ± 0.5^{c} |
| C281A | 3.36 ± 0.37 | 40 ± 9^{b} | 130 ± 12^{b} |
| C281S | 2.29 ± 0.40 | 56 ± 0.6^{b} | 98 ± 11 |
| C367A | 8.17 ± 0.32 | 85 ± 12 | 90 ± 10 |
| C367S | 5.37 ± 0.21 | 79 ± 10 | 91 ± 1 |

^{a}All values were significantly different than wild-type (p ≦ 0.05) except for C367S.
^{b}Significantly different from wild-type (p ≦ 0.05)
^{c}Significantly different from wild-type (p ≦ 0.005)

TABLE VI
Michaelis-Menton Constants for double Cys-Mutants of seHAS
Kinetic analyses were performed as described herein using
membranes prepared from E. coli SURE cells expressing
the indicated seHAS variants.

| | | Km for | Km for | |
|---|---|---|---|---|
| seHAS | Vmax | UDPGIcUA | UDPGIcNAc | Hill |
| MUTANT | (nmol/pmol/h)^{a} | (μM) | (μM) | number |
| WT | 5.60 ± 0.48 | 77 ± 5 | 74 ± 7 | 1.1 ± 0.1 |
| (C226, 262A) | 0.18 ± 0.01^{c} | 134 ± 27^{b} | 650 ± 66^{c} | 1.8 ± 0.2^{c} |
| (C226, 281A) | 1.51 ± 0.42 | 53 ± 5 | 108 ± 4^{b} | 1.0 ± 0.1 |
| (C226, 367A) | 0.84 ± 0.03^{c} | 79 ± 19 | 149 ± 13^{b} | 1.1 ± 0.1 |
| (C262, 281A) | 2.52 ± 0.19 | 113 ± 40 | 298 ± 39^{c} | 1.5 ± 0.1^{c} |
| (C262, 367A) | 4.20 ± 0.44 | 121 ± 3^{b} | 172 ± 3^{c} | 1.4 ± 0.2^{b} |
| (C281, 367A) | 3.69 ± 0.42 | 65 ± 24 | 131 ± 1^{b} | 1.2 ± 0.2 |

^{a}All values were significantly different than wild-type (p ≦ 0.05)
^{b}Significantly different from wild-type (p ≦ 0.05)
^{c}Significantly different from wild-type (p ≦ 0.005)

TABLE VII
Michaelis-Menton Constants for triple and quadruple Cys-Mutants
of seHAS Kinetic analyses were performed as described herein
using membranes prepared from E. coli SURE cells expressing
the indicated seHAS variants.

| | | Km | Km for | |
|---|---|---|---|---|
| | Vmax | for UDPGIcUA | UDPGIcNAc | Hill |
| MUTANTS | (nmol/pmol/h)^{a} | (μM) | (μM) | number |
| WT | 5.60 ± 0.48 | 77 ± 5 | 74 ± 7 | 1.1 ± 0.1 |
| (Δ3C)C²²⁶ | 2.57 ± 0.22 | 81 ± 11 | 273 ± 21^{c} | 1.7 ± 0.3^{b} |
| (Δ3C)C²⁶² | 2.63 ± 0.06 | 87 ± 15 | 189 ± 34^{b} | 1.5 ± 0.2^{b} |
| (Δ3C)C²⁸¹ | 0.08 ± 0.02 | 109 ± 7^{b} | 453 ± 137^{b} | 1.9 ± 0.4^{c} |
| (Δ3C)C³⁶⁷ | 0.78 ± 0.02 | 120 ± 12^{b} | 444 ± 46^{c} | 1.8 ± 0.5^{b} |
| Cys null | 0.95 ± 0.05 | 210 ± 46^{b} | 447 ± 31^{c} | 1.6 ± 0.1^{c} |

^{a}All values were significantly different than wild-type (p ≦ 0.05)
^{b}Significantly different from wild-type (p ≦ 0.05)
^{c}All values were significantly different from wild-type (p ≦ 0.005)

Enzymatic Analysis of seHAS Cys-mutants. To determine which sub-activities of seHAS might be altered by mutating its Cys residues, kinetic analyses of the wild-type enzyme and all the Cys-mutants were performed and their respective Kₘ and Vₘₐₓ values calculated (Tables V-VII). A comparison of the Vₘₐₓ values for each of the single, double, and triple Cys-to-Ala mutants of seHAS verified that the least active mutants were C(226,262)A and seHAS(Δ3C)C²⁸¹, with only ˜1-3% of the wild-type activity (as suggested by the results in FIG. 5). The seHAS(C226S) mutant had ˜10% of the wild-type activity (Table V). The C226A, C(226,367)A, seHAS(Δ3C)C³⁶⁷ and Cys-null mutants had activities between 17-30% of wild-type. The remaining eight seHAS Cys-mutants retained 40% or more of the activity of wild-type seHAS. The only mutant (Table V) that had a higher activity than wild-type was seHAS(C367A).

The Kₘ values for UDP-GlcUA for all the Cys-mutants (Tables V-VII) differed by no more than 2-3 fold from wild-type seHAS. For most of the Cys-mutants, the Kₘ values for UDP-GlcNAc also did not change dramatically (within 1-3 fold). These relatively modest changes indicate that the altered Cys residues in these seHAS variants play a relatively minor role in how the enzyme binds and uses each nucleotide-sugar. However, some combinations of Cys-mutations had more dramatic effects on nucleotide-sugar utilization. For example, the K_{UDP-GlcNAc} value for seHAS(Δ3C)²²⁶ was ˜4-fold higher (Table VII). The K_{UDP-NAc} values for the C(226,262)A mutant (Table VI) and the seHAS(Δ3C)C²⁸¹, seHAS(Δ3C)C³⁶⁷ and Cys-null mutants (Table VII) were even more affected; they were ˜6-9 fold more than wild-type. These latter mutants were clearly less efficient in their utilization of UDP-GlcNAc than the wild-type seHAS. Interestingly, these mutants also had Hill numbers >1.5, compared to a value of 1.0 for the wild-type enzyme, indicating that they had acquired a new level of cooperativity in their utilization of UDP-GlcNAc. All of the above kinetic results indicate a potentially important, though not absolutely essential, role for Cys²²⁶ and Cys²⁶² in seHAS activity.

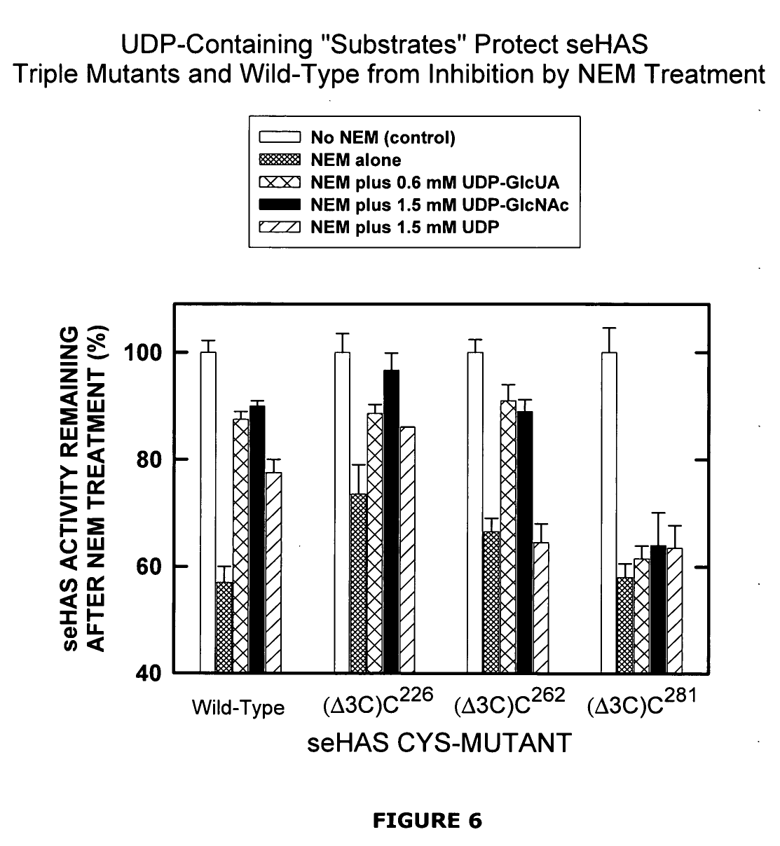

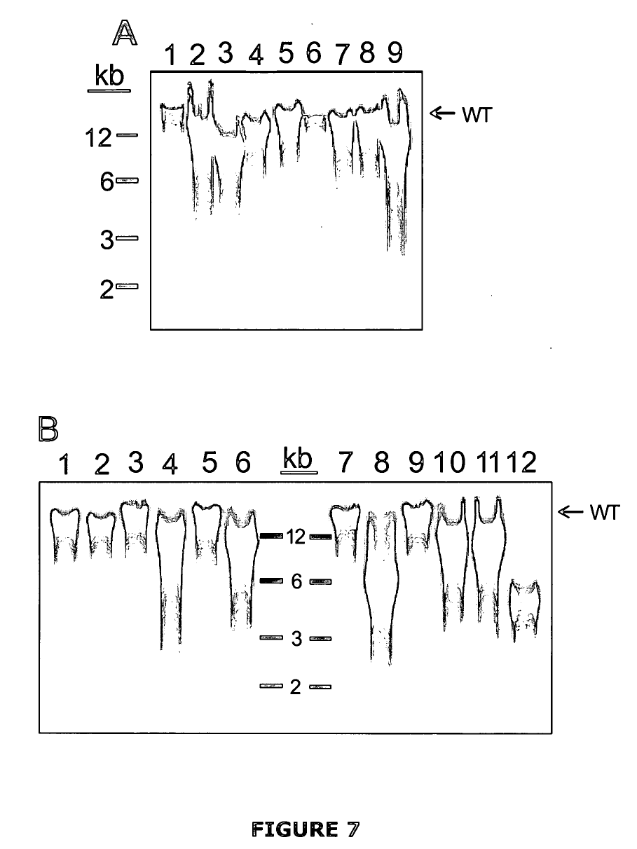

Cysteine residues are not essential for HAS activity but are in or near substrate binding sites. Although the 4 Cys residues in seHAS (positions 226, 262, 281 and 367) are roughly conserved in all Class I HAS family members, the Cys-null mutants of seHAs and spHAS are active, with minimal changes in their kinetics compared to wild type. It is also shown here that neither HAS contains disulfide bonds. To understand why these four cysteines are, nevertheless, largely conserved within the Class I HASs, NEM sensitivity was examined (i.e., the time- and dose-dependent inhibition) of all possible Cys-to-Ala mutants of seHAS. Chemical modification studies of seHAS showed that Cys226, Cys262 and Cys281 react with NEM, whereas Cys367 is not accessible. Substrate protection studies comparing the wild-type and Cys-mutants indicated that NEM-reactive Cys226 and Cys262 are located in or near a substrate binding site(s), because the presence of UDP-GlcUA or UDP-GlcNAc prevented inactivation by NEM. Cys281 appears not to be within a UDP-sugar binding site, since the triple Cys-mutant seHAS (Δ3C)C281 was not protected from NEM inhibition by UDP, UDP-GlcUA or UDP-GlcNAc (FIG. 6). Since sodium arsenite, which can crosslink two close Cys residues, similarly inhibited the double Cys-mutant seHAS C(226,367)A and wild type-seHAS, it is believed that Cys281 and Cys262 are very close (essentially vicinal) in the protein.

Relative size distributions of HA synthesized by various Cys-mutants of seHAS. HASs from different species synthesize HA products with a characteristic, and often different, distribution of sizes. To determine whether any of the Cys-mutants of seHAS synthesize HA having an altered size distribution, compared to wild-type seHAS, agarose gel electrophoresis was used to fractionate the radiolabelled HA products made by each variant enzyme (FIG. 7). The majority of the single (FIG. 7A) and double Cys-mutants (FIG. 7B) synthesized HA of essentially identical size compared to the wild-type enzyme. The C281A and C367S single mutants and the C(262,281)A and C(281,367)A double mutants made smaller products. Three of the four triple mutants (all except seHAS(Δ3C)C²⁸¹) and the Cys-null mutant made smaller HA products (FIG. 6B). The smallest relative HA size distribution was made by the triple mutant seHAS(Δ3C)C²²⁶. Interestingly, the HA size distributions of the seHAS mutants C(226)S, C(226,262)A and (Δ3C)C²⁸¹ were similar to that of the wild-type enzyme, even though these mutants had the lowest activity (1.4-8% of wild-type), and therefore the lowest HA elongation rates. Overall, these results clearly show that mutations of various combinations of Cys residues cause seHAS to synthesize shorter HA chains than the wild-type enzyme, indicating that Cys residues can influence the HA size distribution made by seHAS.

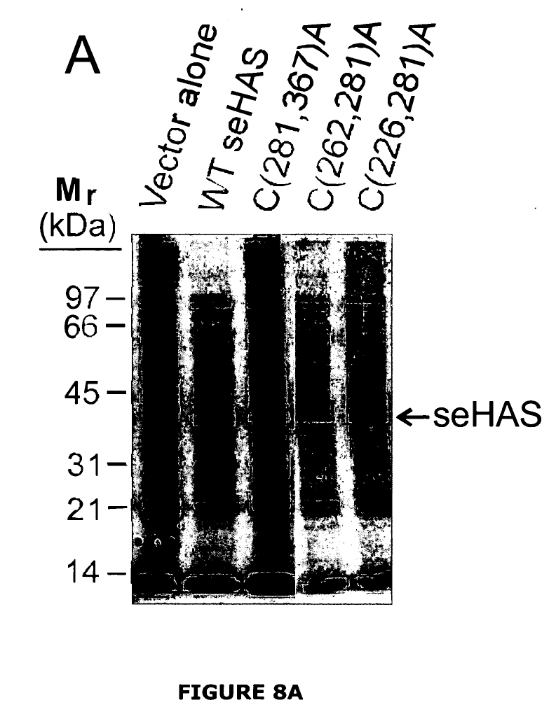

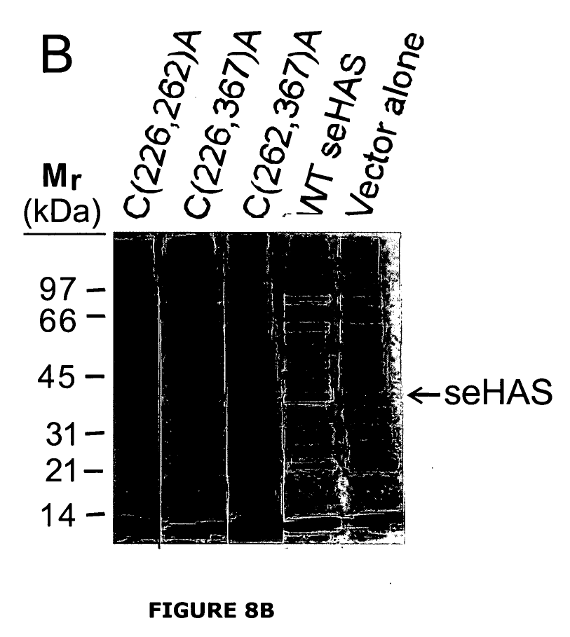

Assessment ofdisulfide bond formation in seHAS. In order to understand the potential role of Cys residues in the function of seHAS, it is necessary to determine if any of its four cysteines are involved in the formation of disulfide bonds. Two approaches were undertaken to answer this question. In the first approach, E. coli membranes containing recombinant seHAS were treated with ¹⁴C-NEM to determine whether the wild-type or Cys-mutant seHAS proteins could be radio-labeled and then identified by autoradiography following SDS-PAGE (FIG. 8). This NEM-reactivity was used to indicate the presence of free cysteines, which are not involved in disulfide bond formation. Each of the six Cys-to-Ala double Cys-mutants of seHAS was radiolabeled by ¹⁴C-NEM. The labeling was specific because the vector-alone control and the Cys-null mutant did not show significant labeling. These results indicate that none of the Cys residues in seHAS are involved in disulfide bonds. A 31 kDa band, which was present in the mixture of NEM-labeled proteins from the wild-type and several double-Cys mutants, could be a degradation product of HAS, since it was not present in the vector-alone controls. Such a fragment is expected to be inactive and illustrates the importance of normalizing the kinetic data to the amount of intact HAS protein, as assessed by protein staining of SDS-PAGE gels.

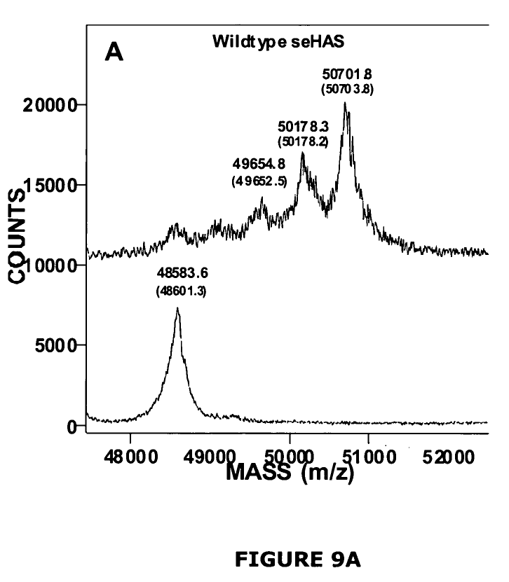

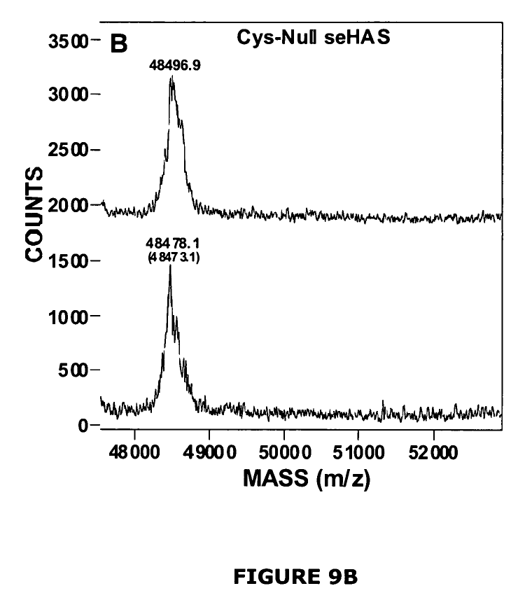

In the second approach to assess the presence of disulfide bonds, the purified enzyme was treated with biotin-PEO-maleimide, and the modified protein products were then analyzed by MALDI-TOF mass spectrometry (FIG. 9). For each biotin-PEO-maleimidyl group added, the mass of the seHAS derivative increased 525.6 Da. The treated wild-type seHAS contained a distribution of derivatized products with increased masses equal to the addition of one-to-four biotin-PEO-maleimide groups per seHAS (FIG. 9A). Most of the proteins were modified by the addition of 3 or 4 groups, demonstrating that the enzyme has no disulfide bonds. The observed mass values for the three largest adducts differed from the predicted values by <0.005%. The degree of modification was only slightly higher when the wild-type seHAS was treated with biotin-PEO-maleimide in the presence of 6 M guanidinium hydrochloride (not shown). This latter result indicates that none of the four Cys residues is substantially buried in the native enzyme; they are all accessible to react with the relatively large modifying reagent. The seHAS^{Cys-null} protein was also treated with biotin-PEO-maleimide, as a control, to verify that no derivitized enzyme products could be formed in the absence of Cys groups (FIG. 9B). The result confirms that the modifying reagent does not react with any other amino acid side chains and is specific for Cys; no covalent adducts were formed with the Cys-null protein.

Mutagenesis of Cys residues and expression of spHAS. To explore the possible presence of disulfide bonds and the functional roles of the conserved Cys residues in the enzymatic activity of the S. pyogenes HAS, each of the six Cys residues in spHAS was mutated to Ser or Ala (see Table IV for a list of the Cys mutants). Subsequently, spHAS mutants with combinations of Cys-to-Ala changes were produced by using site directed mutagenesis or restriction enzyme digestion and ligation of HAS fragments from different mutants. Studies with crude membranes, in which the enzyme activity of spHAS mutants were initially normalized to total membrane protein, indicated that alteration of some Cys residues had a dramatic affect on HA production. For example, spHAS(C225A) appeared to be nearly inactive, and spHAS(C261A) and spHAS(C280A) had less than half the activity of wildtype. However, these initial impressions were incorrect due to significant variations in the expression of spHAS protein among the various mutants.

Therefore, in order to normalize for the level of HAS protein expression, a sensitive and quantitative Western blot-based assay was developed (Heldermon, et al. 2001). Since all of the HAS constructs contain a C-terminal His₆ tag, which is efficiently recognized by a commercial anti-His₅ monoclonal antibody, this antibody was used, after biotinylation, as the primary antibody for analysis of Western blots followed by incubation with ¹²⁵I-streptavidin as the secondary reagent. Unlike standard Western analysis, this detection protocol provides greater sensitivity as well as the ability to quantitate HAS protein over a much broader concentration range. The normalizations for HAS protein expression were performed relative to known amounts of purified spHAS-His₆ included in each analysis as internal standards. Based on the normalized results, it was clear that spHAS(C225S) was expressed at the lowest level relative to any of the other mutants, ˜66% of wildtype (Table VIII). The protein expression levels for the majority of single Cys-mutants were not significantly different than wildtype, although the spHAS(C1 24S) and spHAS(C261 A) variants may have been elevated by ˜35% (p˜0.05). Interestingly, most of the multiple Cys-mutants as well as the Cys-null mutant were expressed at 3-to-5 fold higher levels than the wildtype enzyme. These above differences in relative expression of these spHAS variants were consistent in multiple experiments, with independent cell growth and enzyme induction, indicating that several of the Cys residues in spHAS, particularly the conserved Cys at position 225, may influence the initial folding and stability of the enzyme.

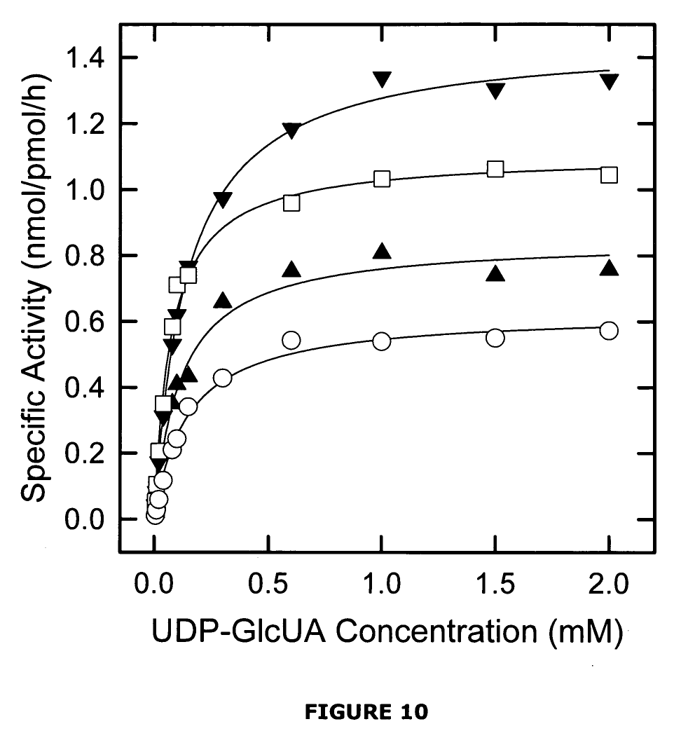

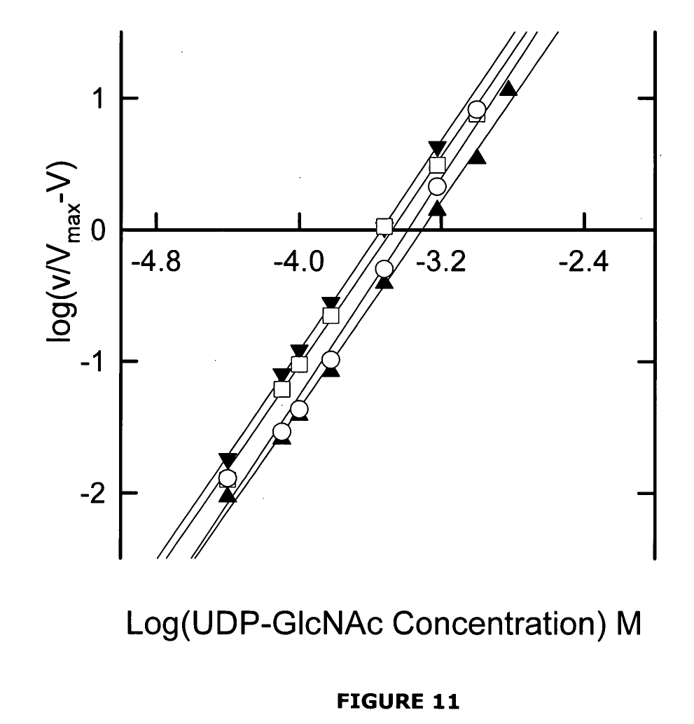

Enzymatic Analysis of Mutants. Kinetic analyses of the single and multiple spHAS Cys-mutants were performed to investigate the possibility that multiple Cys residues are critical in a coordinated way for enzyme activity. The activity of each of the mutants was assayed and normalized by the above method to determine its maximum velocity (Vₘₐₓ) and Michaelis-Menton (Kₘ) constants for UDP-GlcUA and UDP-GlcNAc (FIGS. 10 and 11; Table IX). This analysis revealed no dependence of HAS activity on any single Cys residue. These assays also revealed no extreme changes in maximal enzyme activity relative to wildtype spHAS. The spHAS(C225S) and spHAS(C280A) mutants had the most reduced activities with Vₘₐₓ values at 30-50% of wildtype. The spHAS(C261,280A) and Cys-null mutant had 50-75% of the wildtype activity. Interestingly, spHAS(C124,366,402A) and spHAS(C366A) had an increased activity that was ˜150% of wildtype. The other single mutants, as well as spHAS(C124,402A), and spHAS(C124,261,280,366,402A) demonstrated less than a 25% variation from the wildtype Vₘₐₓ.

All the mutant spHASs were also within 25% of the wildtype enzyme for their K_{UDP-GlcNAc} values. There were no changes in the sigmoidal behavior for UDP-GlcNAc utilization by any of the mutant enzymes. When these data were analyzed using the method of Hill (1913), the Hill numbers were all ˜2 (Table IX), which indicates a high degree of cooperativity associated with the ability of all the mutant enzymes to bind and use UDP-GlcNAc at a fixed UDP-GlcUA concentration. Thus, the cooperativity observed for the utilization of UDP-GlcNAc by the spHAS enzyme (Tlapak-Simmons et al., 1999b) is not influenced by, or dependent on, any of its six Cys residues. Similarly, none of these Cys residues contribute structurally, or otherwise, to a possible secondary binding site for UDP-GlcNAc, i.e., an allosteric binding site.

The K_{UDP-GlcUA} values for all of the single Cys-mutants and the two double Cys-to-Ala mutants were within 50% of wildtype. The remaining multiple Cys- to-Ala mutants exhibited K_{UDP-GlcUA} values that were 2-3 times that of wildtype. Although these multiple Cys-mutations do alter the activity of the enzyme by decreasing the efficiency of utilizing UDP-GlcUA, they do not do so in a large way. Furthermore, the relatively modest difference in activity between the Cys-null mutant and wildtype spHAS clearly shows that cysteine residues are not absolutely necessary for HA synthesis, either catalytically or structurally.

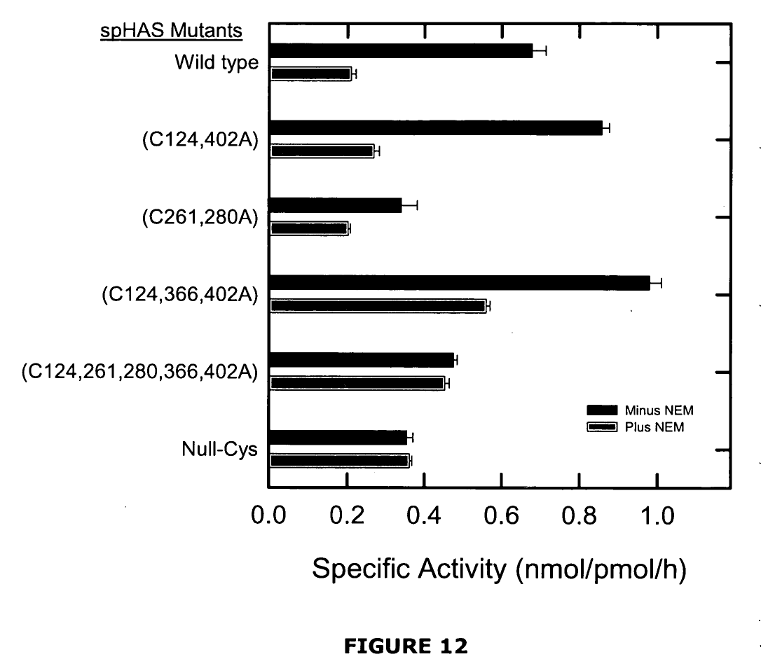

Inhibition of spHAS activity by NEM. NEM treatment of membranes from a panel of multiple Cys-mutants showed that this inhibition was no longer present in spHAS^{Cys-null} or the mutant with only Cys²²⁵ intact, whereas NEM sensitivity remained in the other multiple Cys-mutants (FIG. 12). These results indicate that the inhibition of the wildtype enzyme by NEM or other sulfhydryl/reactive agents is most likely due to modification of the Cys residues alone, rather than the loss of the S-H group. The lack of inhibition of the single Cys-containing mutant demonstrates that Cys²²⁵ is either predominantly inaccessible to modification by NEM due to its position in the enzyme or that this particular cysteine residue is not involved in the inhibition response of the enzyme when modified by NEM.

TABLE VIII
Expression levels of various Cys-mutants of spHAS.
Membranes prepared from E. coli SURE cells expressing
the indicated spHAS variants were fractionated by SDS-PAGE
and the proteins were transferred to nitrocellulose. SpHAS protein
levels were quantitated as described in Methods and the values
were normalized to that of the wildtype. Student t-tests were
performed to assess the significance of differences compared to the
expression of the wildtype enzyme.

| | Relative | Standard | | t-test (p |
|---|---|---|---|---|
| spHAS Construct | Concentration | Deviation | n | value) |
| Wildtype | 1.00 | 0.18 | 8 | — |
| C124A | 1.01 | 0.12 | 4 | 0.89 |
| C124S | 1.35 | 0.39 | 4 | 0.06 |
| C225A | 1.24 | 0.52 | 4 | 0.24 |
| C225S | 0.66 | 0.22 | 4 | 0.02 |
| C261A | 1.35 | 0.10 | 3 | 0.01 |
| C261S | 1.16 | 0.14 | 4 | 0.16 |
| C280A | 1.04 | 0.25 | 4 | 0.73 |
| C280S | 1.30 | 0.42 | 4 | 0.10 |
| C366A | 0.97 | 0.30 | 4 | 0.84 |
| C366S | 1.11 | 0.24 | 4 | 0.38 |
| C402A | 1.05 | 0.07 | 2 | 0.75 |
| C402S | 0.86 | 0.14 | 4 | 0.21 |
| C(124, 402)A | 0.82 | 0.33 | 6 | 0.21 |
| C(261, 280)A | 3.20 | 1.48 | 6 | <0.001 |
| C(124, 366, 402)A | 3.61 | 1.72 | 6 | <0.001 |
| C(124, 261, 280, 366, 402)A | 3.98 | 0.50 | 6 | <0.0001 |
| Cys-Null | 4.94 | 1.08 | 6 | <0.0001 |

TABLE IX
Kinetic constants for Cys-to-Ser/Ala mutants of spHAS.
The Vₘₐₓ values are in μmol of both substrates
incorporated per hour per pmol spHAS. The Kₘ values
for UDP-GIcNAc and UDP-GIcUA were determined at 0.5 mM
UDP-GIcUA and 1.5 mM UDP-GIcNAc, respectively. Experiments
were performed in duplicate or triplicate for the single or
multiple Cys-mutants, respectively. For the wildtype, n = 7.
Values that differ significantly, based on the Student t-test, from
that of the wildtype enzyme are indicated by * (p < 0.05)
or ** (p < 0.001).

| | Vmax | Km UDP-GIcUA | Km UDP-GIcUA | Hill |
|---|---|---|---|---|
| spHAS Construct | (μmol/pmol/h) | (μM) | (μM) | Number |
| Wildtype | 791 ± 215 | 50.4 ± 10.8 | 398 ± 112 | 1.8 ± 0.1 |
| C124A | 617 ± 132 | 37.3 ± 10.1 | 458 ± 60 | 1.8 ± 0.1 |
| C124S | 584 ± 176* | 32.7 ± 0.6 | 459 ± 8.0 | 1.8 ± 0.1 |
| C225A | 852 ± 306 | 62.2 ± 4.1 | 372 ± 26.5 | 1.8 ± 0.1 |
| C225S | 366 ± 37** | 42.7 ± 4.6 | 464 | 1.7 |
| C261A | 923 ± 173 | 42.6 | 454 ± 6.5 | 2.0 ± 0.1 |
| C261S | 764 ± 103 | 45.6 | 499 ± 7.0 | 1.9 ± 0.1 |
| C280A | 264 ± 147** | 33.8 ± 9.7 | 344 ± 45.5 | 1.7 ± 0.1 |
| C280S | 680 ± 90 | 29.4 ± 1.6* | 325 ± 9.0 | 1.8 ± 0.1 |
| C366A | 1201 ± 154** | 56.8 ± 12.8 | 397 ± 1.5 | 1.8 ± 0.0 |
| C366S | 721 ± 121 | 34.1 ± 2.9 | 472 ± 4.5 | 1.8 ± 0.0 |
| C402A | 969 ± 163 | 43.8 ± 9.2 | 427 ± 20.5 | 1.8 ± 0.0 |
| C402S | 774 ± 98 | 44.4 ± 9.4 | 443 ± 35.5 | 1.8 ± 0.0 |
| C(124, 402)A | 894 ± 306 | 39.5 ± 7.0 | 330 ± 38.8 | 1.9 ± 0.1 |
| C(261, 280)A | 436 ± 105* | 54.8 ± 36.7 | 389 ± 20.8 | 1.9 ± 0.1 |
| C(124, 366, 402)A | 1247 ± 127** | 130 ± 21.6** | 285 ± 4.5 | 1.8 ± 0.2 |
| C(124, 261, 280, | 702 ± 112 | 101 ± 24.1* | 471 ± 20.8 | 1.9 ± 0.1 |
| 366, 402)A | | | | |
| Cys-Null | 522 ± 47* | 153 ± 39.3** | 450 ± 40.7 | 1.8 ± 0.1 |

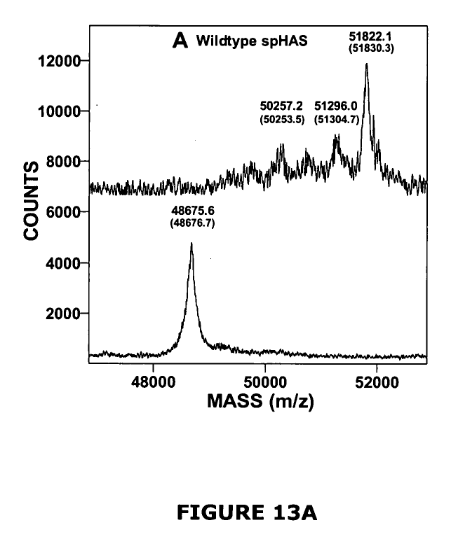

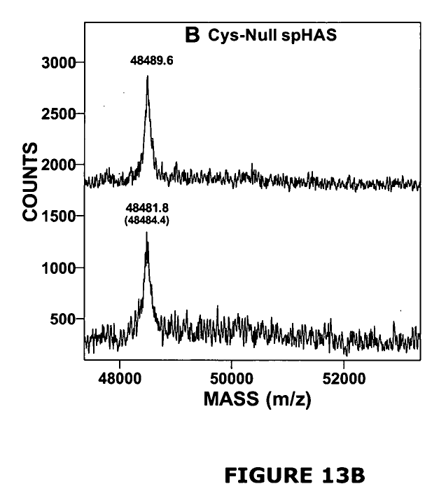

Assessment of disulfide bond formation. Although Cys residues may not be required for the enzymatic activity of the HAS proteins, they could still be important in the structural integrity and long-term stability of the enzyme as indicated by the reduced expression of the spHAS(C225S) mutant and the increased expression of the spHAS(C124S) and spHAS(C261A) mutants (Table VIII). Cys residues may also be important for maintaining the proper enzyme conformation to allow extrusion of the growing HA chain through the membrane. The primary manner in which Cys residues play structural roles in proteins is by forming either inter- or intra-molecular disulfide bonds. To investigate the possibility of disulfide bonds in spHAS, a chemical labeling approach was utilized to determine the number of Cys residues that are free and, therefore, could not be involved in disulfide bonding. Biotin-PEO-maleimide was allowed to react with purified spHAS, while bound to a Ni⁺²-loaded NTAcolumn, and the modified protein products were then analyzed by MALDI-TOF mass spectrometry (FIG. 13). In nondenaturing conditions, treated wildtype spHAS samples revealed a distribution of derivatized products with increased masses equal to the addition of from one-to-six biotin-PEO-maleimide groups per spHAS, with the majority of the protein being modified by the addition of 5 or 6 groups (FIG. 13A). As a control for this chemical modification approach, samples of spHAS^{Cys-null} were treated with biotin-PEO-maleimide in the same way to verify that no derivitized enzyme products would be formed in the absence of Cys groups. The result (FIG. 13B) demonstrates that no covalent adducts form with the Cys-null protein, which confirms that the modifying reagent is specific for Cys and does not react with any other amino acid side chains. Treatment of the wildtype or Cys-null spHAS proteins with biotin-PEO-maleimide in the presence of 6 M guanidinium hydrochloride gave essentially the same results as obtained in the absence of the denaturing agent, although the degree of modification was slightly greater (not shown). This latter result indicates that spHAS contains no weak disulfide bonds that might be susceptible to reversible reduction when the protein is denatured. The overall results demonstrate that there are no disulfide bonds in the wildtype spHAS enzyme, and that there is a mixed degree of exposure of the six Cys residues in this protein to the biotin-PEO-maleimide reagent in solution.

Mutant HAS HA size range and prevalent size of HA. Because spHAS mutants in which C280 was substituted with alanine synthesized HA of a lower weight average mass than the spHAS wild type enzyme, other amino acid substitutions were also examined (Table X). For example, the C280T and C280V mutants made HA with a larger size distribution than the wild type, whereas the C280G mutant made HA that had a smaller weight average mass than wild type.

TABLE X
Mutant HAS HA Size Range and Prevalent Size
HA produced by mutants of spHAS and seHAS
in one hour at saturating substrate concentration were
run on 1% agarose gels. Reactions were radiolabeled
by including UDP-[¹⁴C]GlcUA in the reaction mix and
HA product distribution was assessed with a Molecular Dynamics
Phosphoimager. Size was estimated relative to High Molecular
Weight and Kb DNA ladder standards. Distribution of product
is reported as the percentage of total counts (IDV) between
various size ranges. The prevalent HA size produced
(“Peak Size”) is reported as the approximate MW
in MDa at which the greatest IDV intensity was located.
MW ranges are shown as MDa and kbp of DNA in parentheses.
C226A, C262A, C281A, and C367A are mutants of seHAS.

| | | <2 | 2-4 MDa | 4-6 MDa | >6 MDa |
|---|---|---|---|---|---|
| Mutant | Peak Size (MDa) | MDa (<3 Kb) | (3-6 Kb) | (6-9 Kb) | (9->48.5 Kb) |
| WT (spHAS) | 2-3 | 33 ± 5 | 29 ± 8 | 12 ± 2 | 26 ± 8 |
| C280A | ˜1 | 65 ± 12 | 15 ± 2 | 5 ± 3 | 15 ± 7 |
| C280G | ˜1 | 58 ± 10 | 17 ± 5 | 5 ± 2 | 19 ± 4 |
| C280S | 3-4 | 27 ± 4 | 40 ± 3 | 13 ± 3 | 21 ± 3 |
| C280T | 8-32 | 14 ± 2 | 27 ± 2 | 14 ± 3 | 44 ± 4 |
| C280V | 4-5 | 26 ± 3 | 40 ± 2 | 14 ± 3 | 21 ± 6 |
| C124S | ˜2 | 35 | 37 | 13 | 15 |
| C124A | ˜2 | 31 | 33 | 17 | 19 |
| C225S | 3-4 | 33 | 45 | 14 | 8 |
| C225A | 5-6 | 16 | 29 | 23 | 32 |
| C261S | 5-6 | 18 | 31 | 22 | 29 |
| C261A | 4-5 | 22 | 35 | 20 | 23 |
| C366S | ˜2 | 38 | 36 | 11 | 14 |
| C402S | 2-3 | 25 | 36 | 17 | 22 |
| C402A | ˜2 | 34 | 37 | 12 | 17 |
| C402A | ˜2 | 37 | 35 | 12 | 16 |
| C124, 402A | 1-2 | 38 | 0 | 12 | 20 |
| seHAS | 1-2 | 42 ± 21 | 25 ± 7 | 14 ± 5 | 19 ± 9 |
| C226A | ˜5 | 12 ± 1 | 38 ± 2 | 23 ± 0 | 27 ± 1 |
| C262A | 1-2 | 48 ± 0 | 28 ± 1 | 9 ± 0 | 15 ± 1 |
| C281A | 1 | 55 ± 1 | 26 ± 1 | 5 ± 1 | 14 ± 1 |
| C367A | ˜3 | 15 ± 1 | 62 ± 0 | 4 ± 1 | 19 ± 2 |

The HAS enzymes are unique in that they polymerize two sugars, GlcUA and GlcNAc, in an alternate fashion and simultaneously extrude the growing HA chain through the plasma membrane. The streptococcal HASs are the smallest members of the Class I HAS family, and perform all the functions required for HA synthesis and secretion from cells. Unlike the eukaryotic HAS enzymes, with which they share substantial homology and probably an identical topological organization in their common regions, the streptococcal enzymes have been easier to study because they can be readily overexpressed, purified and characterized. To date, only one eukaryotic enzyme, mouse HAS1, has been overexpressed, purified and characterized kinetically.

The importance of Cys residues in seHAS and spHAS was initially focused on for three reasons. First, Cys residues play important structural and functional roles in many proteins (e.g., 35). Second, the four Cys residues in seHAS at positions 226, 262, 281, and 367 are completely conserved in the two other streptococcal enzymes, suHAS and spHAS, and are generally conserved in all the other eukaryotic HASs (FIG. 1). Finally, p-chloro-mercurobenzoate had been reported to inhibit HA biosynthesis by the Group A spHAS in a cell-free system (Sugahara et al., 1979). Although no further studies on the role of sulfhydryls in HAS function had appeared since that report, it was important to investigate the possibility that Cys residues may be required for HAS activity.

The present results demonstrate that a variety of sulfhydryl reagents inhibit both the spHAS and seHAS enzymes. This inhibition could reflect an important role of Cys in the function of these bacterial HAS proteins. However, interpretation of these results is complicated by the fact that Cys-modification creates two changes in the enzyme; the S-H group is eliminated but a new S-R group is also introduced, where R depends on the sulfhydryl reagent used. Because all the R groups are larger than the initial H, modified Cys residues may create new steric constraints for particular enzyme functions such as substrate binding. Alternatively, different degrees of HAS inhibition by different sulfhydryl reagents could indicate their different reactivity towards Cys residues, which would depend upon their size, charge or polarity. The use of site directed mutagenesis to alter the native Cys residues, while subject to the same concerns noted above, provides a complementary approach to determine the importance of Cys residues in HAS function. Both approaches show that although HAS activity is decreased by altering Cys residues, it is not eliminated; the completely modified seHAS and spHAS Cys-null enzymes were still able to perform all of the functions needed for HA synthesis.

The experiments and results detailed herein demonstrate clearly that neither the seHAS enzyme nor the spHAS enzyme contains any disulfide bonds. It is reasonable to conclude, therefore, that the streptococcal HAS proteins do not have disulfide bonds. It may be more difficult to determine if the eukaryotic HAS proteins contain disulfide bonds, since these proteins are difficult to purify in high yield and contain more Cys residues (≧14) than the streptococcal proteins.

All HAS enzymes make a broad size range of HA, rather than a discrete size. This heterogeneity of product size may be important biologically for particular functions of the three vertebrate HAS enzymes. In addition, the HA size distribution made varies among the streptococcal HASs and also among the three mammalian HAS isoforms. These enzymatic differences in the size distribution of HA products, which have only been observed in vitro (e.g., in isolated cells or membrane preparations), could have very significant biological consequences if they also occur in vivo in various eukaryote cells and tissues. Numerous studies during the last decade have demonstrated that HA is not simply a structural component of the extracellular matrices of most vertebrate tissues, but also a cell signaling molecule capable of modifying important aspects of cell behavior, including migration and adhesion. The most interesting and surprising aspect of this new paradigm regarding the biological functions of HA is that many cells respond only if the HA is a specific size. In particular, small oligosaccharides of HA have very different biological activities than large, native-size HA.

An intriguing finding of the present invention is that some, but not all, combinations of Cys-mutations in seHAS cause the enzyme to synthesize HA products having an altered size. Eight of the 19 Cys-mutants examined synthesized HAwith an apparently normal distribution of sizes that were shifted to varying degrees to smaller mass. There was no apparent correlation between changes in HA elongation rate (Vₘₐₓ values) and HA size distribution among these Cys-mutants. The least active seHAS variants, nonetheless, made HA products that were similar in size to the HA made by the wild-type enzyme. The agarose gel electrophoresis technique is very suitable for obtaining a qualitative assessment of size differences. However, it is very difficult to assess the reliability of size assessments outside the narrow range in which migration is proportional to size. For example, several Cys-mutants (e.g., one each of the single, double and triple mutants in FIG. 7) may actually synthesize substantially larger HA than wild-type but the migration differences compared to wild-type are very small. For these reasons, a study to characterize the HA size distributions of these seHAS mutants was undertaken using gel permeation chromatography coupled to dynamic light scattering (MALLS). The present invention demonstrates a role of Cys residues in controlling HA chain length. In particular, the single seHAS Cys-mutant C281A makes much smaller HA, whereas the seHAS C281S mutant makes HA products very similar in size, compared to wild-type seHAS. In addition, the spHAS Cys mutant C280A also makes much smaller HA, as seen in patent application US Publication No. US2003/0082780 A1, published May 1, 2003, the contents of which have been previously incorporated herein by reference in their entirety.

Although NEM treatment of seHAS caused the velocity maximum (Vₘₐₓ) of the enzyme to decrease, it did not substantially change the Kₘ values for either nucleotide-sugar compared to untreated seHAS. These results indicate that the ability of the NEM-treated enzyme to bind each substrate is not greatly decreased by modification of its Cys residues, but the overall catalytic rate is slowed. In contrast, some of the site specific Cys-mutants showed greater changes in their kinetic constants.

None of the single Cys-to-Ala or Cys-to-Ser mutants of seHAS or spHAS are inactive, indicating that no single Cys residue plays a critical, necessary role in HA synthesis. The specific HAS activity remaining in the single, double, triple, and Cys-null mutants confirms that Cys is not required at any position within the enzyme for a critical step in HA synthesis. Nonetheless, Cys²²⁶ and Cys²⁶² together appear to play an important role in the activity of seHAS, since the double mutant seHAS(C226,262A) was the least active Cys-mutant with only 2-3% of the wild-type activity. Despite its low activity, this double mutant, nonetheless, synthesized HA of normal size. The triple Cys-mutant seHAS(ΔC3)C²⁸¹ also had very low activity, similar to the double Cys-mutant seHAS(C226,262A), and also synthesized normal size HA. These results indicate that alteration of Cys³⁶⁷ does not cause decrease in HAS activity and is consistent with the single Cys-mutant results in Table IX. In fact, the seHAS C367A and spHAS C366A mutants both exhibited increases in activity over the corresponding wild-type enzymes.

Interestingly, the lower functionality of seHAS(C226,262A) was substantially relieved by the introduction of a C281A change to create the triple Cys-mutant seHAS(ΔC3)C³⁶⁷. Possibly, a structural or functional constraint, perhaps related to HA chain length, brought about by mutating Cys²²⁶ and Cys²⁶² to Ala is substantially relieved by simultaneously mutating Cys²⁸¹. The triple mutant seHAS(ΔC3)C³⁶⁷ and the Cys-null mutant had similar activities and HA product sizes, suggesting a similar degree of compensation for the otherwise deleterious Cys²²⁶/Cys²⁶² double mutation. The Cys-null mutant of seHAS retained approximately 20% of wild-type activity. The results indicate that Cys²²⁶ and Cys²⁶² play an important role in the overall activity and kinetic characteristics of seHAS, but Cys²⁸¹ may play a role in regulating HA size. Based on the recently determined topology of spHAS and its high level of homology with seHAS (72% identical plus 10% similar residues), we know that Cys²²⁶, Cys²⁶², and Cys²⁸¹ are present in the central domain region of seHAS (FIG. 1), which is the region that contains β-glycosyltransferase family motifs. The topological model predicts that Cys³⁶⁷ is very close to transmembrane domain 4 and is probably not near the glycosyltransferase motifs.

Based on the NEM-modification and Cys-mutagenesis results, it appears that one or more Cys residues may be located close to the nucleotide-sugar binding sites of the seHAS or spHAS enzymes. This possibility provides a rationale to explain why modification or alteration of these Cys residues interferes with enzyme function and lowers enzyme activity. Preliminary results suggest that either substrate, UDP-GlcUA or UDP-GlcNAc, can protect seHAS from inhibition by NEM (FIG. 6), supporting the premise that at least one Cys residue is located in or near a nucleotide-sugar binding pocket. Substrate binding to this site appears to interfere with the reaction between NEM and the nearby Cys residue(s). Similar conclusions about the proximity of Cys residues to substrate binding sites have been reported for several other proteins, including the lactose permease, glutathione synthetase, glucocorticoid receptor, retinoic acid receptor β, and plasma membrane proton-ATPase. All of these studies found that modification of Cys residues by sulfhydryl reagents decreased the activity of the protein, even though Cys mutagenesis did not inactivate the protein. Another recent study generated 400 Cys-scanning mutants of a tetracycline transporter in order to map the membrane topology and active site of the protein in membrane preparations. The ability of tetracycline to protect only particular Cys residues from reaction with NEM, and subsequent inactivation of the protein, allowed the tetracycline binding site and channel to be mapped.

Another important consideration in evaluating the importance of Cys residues in spHAS, and the other Class I HAS family members in general, may be their involvement in HA translocation. The mechanism by which these enzymes are able to hold onto the growing HA chain, while they continuously extrude the polysaccharide through the bacterial cell or plasma membrane, is still unknown. This extrusion process is referred to as a translocation, since the HA is not completely transferred across the membrane and released as would occur in a typical transport process. The synthesis and extracellular accumulation by some bacteria of polysaccharides, such as polysialic acid, often requires multiple factors and proteins encoded by very complex multi-gene operons (Moxon and Kroll, 1990; Bliss and Silver, 1996). In contrast, all of the genetic and biochemical evidence to date (reviewed in Weigel, 1998) demonstrate that the streptococcal enzymes are able to initiate HA chain formation and then rapid extension of the HA chain in the absence of any primer or other proteins. Other than the two sugar nucleotide substrates and Mg⁺², the purified spHAS or seHAS enzymes only require a phospholipid (Tlapak-Simmons et al., 1999a) in order to produce high molecular weight HA (>10⁶ Da). In particular, cardiolipin dramatically stimulates the specific activity of detergent solubilized or purified spHAS and seHAS. The size distribution of HA products is very similar for enzyme in isolated membranes or after solubilization with dodecylmaltoside and affinity purification (data not shown). Therefore, the presence of a natural intact phospholipid bilayer and membrane does not affect the ability of the HAS enzymes to synthesize HA. Presently, a suitable assay to evaluate the ability of the wildtype or Cys-mutant enzymes to translocate HA is not available.

The creation of seHAS^{Cys-null} and/or spHAS^{Cys-null} mutants that retain enzymatic activity enables a more in depth analysis of the tertiary structure of the HAS enzyme and conformational changes that occur during substrate binding, catalysis or HA translocation. To understand these processes, it is necessary to determine the interactions and molecular proximity of various domains within the protein in a more defined way. Cys-scanning mutants of seHAS or spHAS containing a single unique Cys residue at a desired position could enable one to employ electron paramagnetic resonance studies by modifying this Cys residue with a suitable probe. This approach, for example, allowed Voss et al., to determine the proximity of that modified residue to another region of the Lac permease. Similarly, chemical modification of a single unique Cys residue with a fluorescent probe enables a systematic analysis of the localized environment within different regions of the protein. Interacting or proximal domains within seHAS or spHAS may also be determined by assessing the formation of disulfide bonds in specific mutants containing two Cys residues. Such approaches help to elucidate the structure and function of seHAS and increase our understanding of how the HAS family is able to synthesize HA.

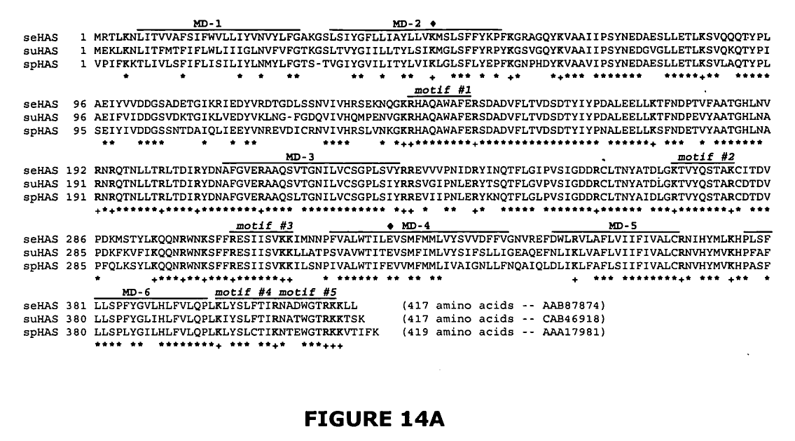

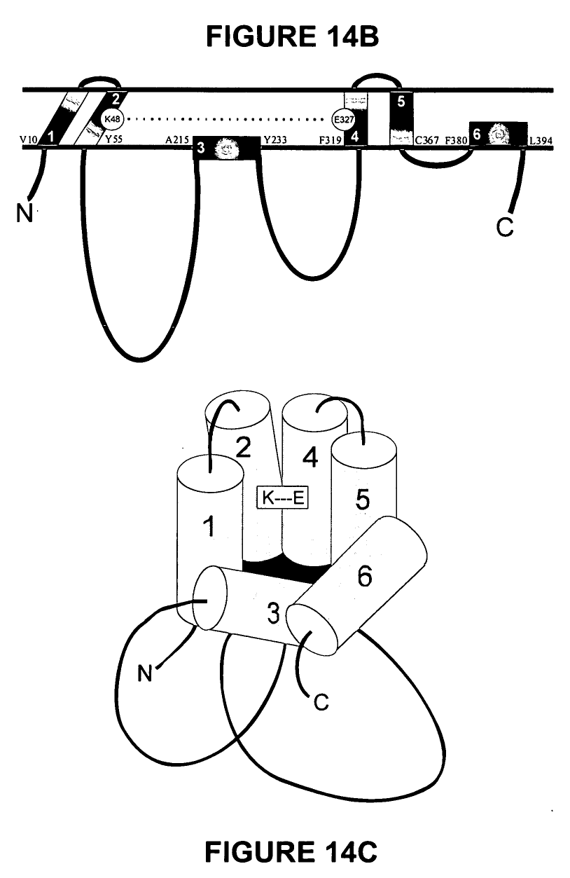

Targeted mutagenesis within two membrane domains of seHAS and generation of the “double switch” mutant. There is a paradox in understanding how HASs polymerize long HA chains. HA chains must be free to move through the enzyme at rapid rates as they are elongated. Thus, one expects that the HA-binding ability of HAS would not be a “high affinity” interaction, since a low off-rate would hinder chain movement (i.e., translocation though the membrane). Yet, HA-binding “affinity” cannot be very low because some chains are held for >10,000 cycles of HA disaccharide assembly and translocation before large HA chains (˜4×10⁶) are released. It is expected that HAS possesses multiple HA-binding regions whose interactions with the growing polymer must be made and broken in a coordinated manner to enable the growing HA chain to translocate within the enzyme without being released prematurely. FIG. 14A illustrates 5 motifs that are putative HA-binding motifs of the type B-X₇-B, where B is a basic amino acid (i.e., Arg or Lys) and X is any amino acid, as described by Yang et al., 1994. It is anticipated that HAS utilizes two types of HA-binding regions; one type that holds and orients the donor HA-UDP chain for assembly of the next disaccharide unit and one type that holds the growing HA chain when it is released from the former site(s) and is translocated through the membrane.

Although there is no information about the possible HA-binding regions within HAS, it is intriguing that motifs #2-5 are absolutely conserved in the strepHASs and are generally conserved at these positions in the Class I HAS family (i.e., conserved within the same general region of the primary sequence). In addition, although motif #1 in suHAS contains Gln, rather than Arg/Lys (which could still H-bond with HA), it is still a good candidate motif, because partial or “weaker” HA-binding motifs must be considered for the reasons noted above. An overlapping motif at position #1 is highly conserved in the Class I family. Further, complete conservation of HA-binding regions within the Class I family are not expected because the three human HASs intrinsically make HA of different sizes; this reflects the different abilities of HAS1, HAS2 and HAS3 to retain their HA chains. In particular, motif #4 (with Asn rather than RIK) and motif #5 are conserved in all HASs, except the chlorella enzyme.

The topological organization of spHAS and probably all Class I HASs is shown in FIG. 14B. The experimentally determined topology of spHAS is similar to that initially predicted except for two membrane domains (MD3 and MD6) that are not transmembrane domains (TMDs). The N- and the C-termini and the large central domain are intracellular. The first two TMDs are β-sheets (not α-helices) and create a small extracellular loop that is inaccessible to proteases. MD3 within the large internal central domain is associated with, but does not traverse, the membrane.

There are then two TMDs connected by a second small extracellular loop that is also inaccessible to proteases. MD6, which has amphipathic helices, is within the C-terminal 50 amino acids of spHAS and does not cross the membrane. Numerous Mds may be required for HAS to create a pore-like structure through which a growing HA chain can be extruded to the exterior. Based on their similarities, all Class I HASs should have similar topological organizations of their spHAS-related domains.

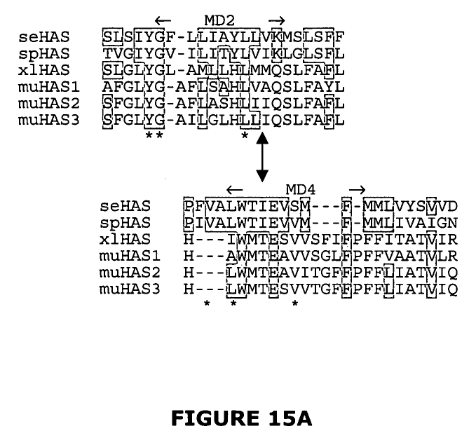

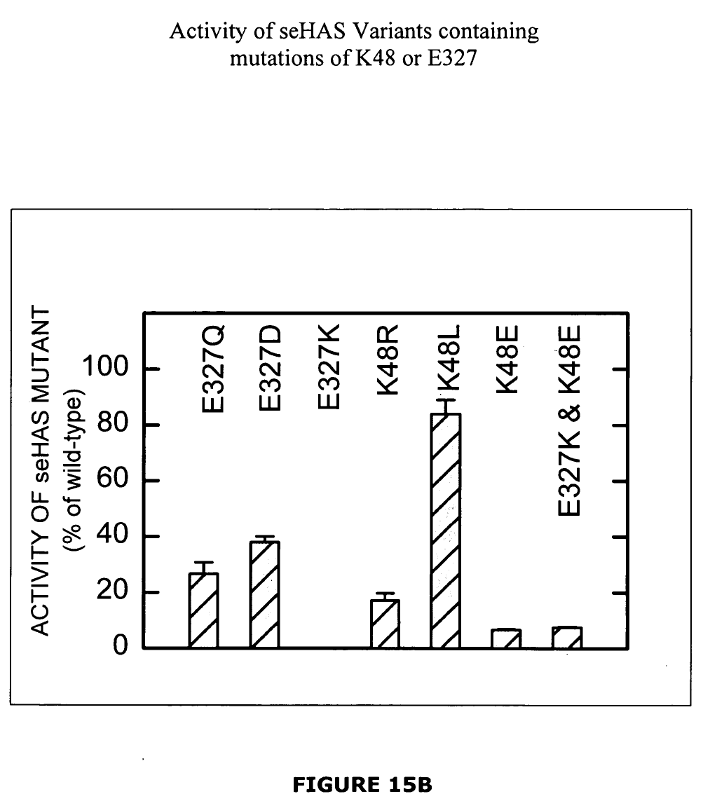

The presence in all Class I HASs of a possible charge/polar-pair between two membrane domains was noted, and in seHAS, these residues are K48 and E327 (indicated by the ovals in FIG. 14B and as seen in FIG. 15). It is believed that the two charged or polar residues form a salt bridge or strong H-bond link that anchors MD2 (which traverses the membrane from outside to inside) and MD4 (which traverses the membrane from inside to outside).

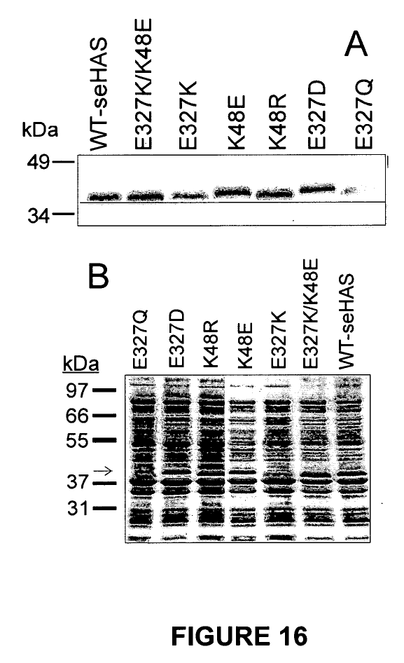

To examine the possibility that K48 and E327 interact or are otherwise important for HAS function, we created mutants in which K48 was changed to Arg or Glu and E327 was changed to Asp, Lys or Gln (Table XI). All of these seHAS mutants were expressed, based on Western analysis (FIG. 16A), although the protein levels varied greatly (FIG. 16B). The E327D, K48R, and K48E variants showed near-wildtype expression levels (FIG. 16B). In contrast, the E327Q and E327K variants were expressed poorly. However, adding the K48E mutation restored expression of the seHAS(E327K) protein to wildtype levels. In subsequent kinetic analyses, different amounts of membrane protein were used to compensate, at least partially, for these expression differences.

TABLE XI
SeHAS K48 and E327 Mutants.

| SeHAS Mutant | SEQ ID NO: |
|---|---|
| E327Q | 93 |
| E327D | 94 |
| E327K | 95 |
| K48R | 96 |
| K48E | 97 |
| E327K + K48E | 98 |

The HAS family members all migrate in SDS-PAGE at a considerably smaller apparent size (DeAngelis et al., 1993; and Weigel et al., 1997), e.g., the ˜49 kDa seHAS migrates as a ˜42 kDa protein. This anomalous behavior indicates that, despite boiling in SDS, these proteins retain substantial secondary or tertiary structure and do not completely unfold. Interestingly, the SDS-PAGE migration rates of seHAS(E327D) and seHAS(K48E) were reproducibly slower than wildtype or the other variants, indicating that these two proteins were more unfolded and more sensitive to denaturation by SDS. Normal migration was restored by adding the E327K change to the seHAS(K48E) protein (FIG. 16A).

All the seHAS variants had synthase activity, although seHAS(E327K) was barely detectable (Table XII). Vmax values, relative to wildtype, ranged from 0.2% for seHAS(E327K) to 38% for seHAS(E327D). Amino acid changes to similar residues at positions K48 or E327 were generally less disruptive than changes to a dissimilar polar residue. The UDP-sugar Km values for the seHAS mutants were not altered drastically; they differed from wildtype by up to ˜2-fold (not shown). Although seHAS variants with E327 changed to Asp or Gln had good activity, the E327K variant had dramatically less activity (Table XII). However, both the activity and expression of seHAS(E327K) were greatly increased by combining this mutation with the reciprocal change at position 48 to create the seHAS(K48E,E327K) mutant. This double-switch variant was expressed as well as wildtype (FIG. 16B) and exhibited 7.4% of wildtype activity. The conversion of K48 to Glu essentially rescued the seHAS(E327K) enzyme, increasing activity of this variant by >46-fold.

TABLE XII
HA Synthase Activities and Molar Masses of HA Products Produced by
seHAS Variants at K48 and E327.
Multiple experiments using 2-3 independent membrane
preparations were performed to analyze Vmax values and
the weight-average molar mass of HA made by membranes containing the
indicated seHAS variants. Values shown are the mean ± standard
error (n 3).

| SeHAS Variant | Vₘₐₓ (% of wild type) | Molar Mass of HA Products |
|---|---|---|
| Wild Type | 100 | 3.64 ± 0.07 |
| E327D | 38 ± 2.0 | 3.20 ± 0.01 |
| E327Q | 27 ± 4.1 | 1.52 ± 0.38 |
| E327K | 0.16 ± 0.2 | Not detected |
| K48E | 6.7 ± 0.2 | 0.68 ± 0.02 |
| K48R | 17.2 ± 2.6 | 1.89 ± 0.10 |
| E327K & K48E | 7.5 ± 0.3 | 0.61 ± 0.09 |

If the K48 and E327 residues in seHAS mediate an interaction between MD2 and MD4, then their disruption could destabilize a putative intramolecular pore, through which the enzyme translocates HA. Perturbing this pore might alterthe rate atwhich HAS could translocate a growing HA chain (resulting in a decreased synthesis rate), and also affect the ability of HAS to retain the growing HA chain, resulting in smaller HA products. These results, therefore, support the idea that K48 and E327 interact with each other and that MD2 and MD4 are close enough for these residues to form an ion-pair or H-bonds.

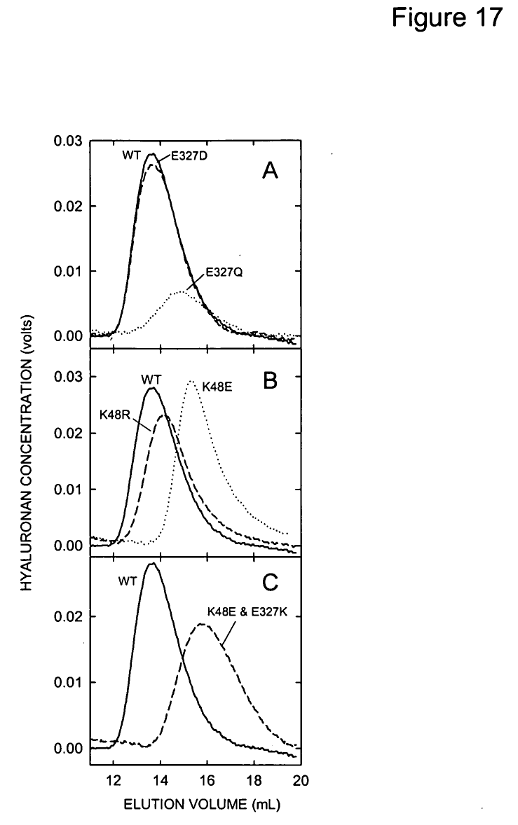

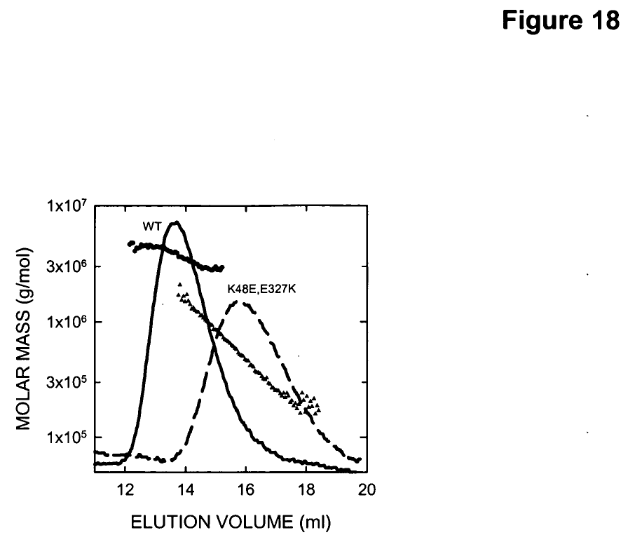

There are no reports using SEC-MALLS to characterize HA made by membranes containing HAS, despite the fact that MALLS is one of the best techniques to assess the size distributions and absolute masses of polydisperse HA. A procedure was recently developed by the inventors to perform such analyses, and this new approach was used to quantify possible differences in HA size made by these seHAS variants (FIGS. 17 and 18). The weight-average molar mass values are summarized in Table XII. The gel filtration profiles show that the distributions of HA products remained normal (i.e., approximately symmetric) even though the mutants made smaller HA (FIG. 17). The seHAS(K48E) and seHAS(K48E,E327K) variants made the smallest HA, with essentially identical molar masses. The weight-average masses of HA products made by the wildtype enzyme and seHAS(K48E,E327K) were 3.64 MDa and 0.61 MDa, respectively (FIG. 18, Table XII). FIG. 18 illustrates the high quality light scattering data that can be obtained for HA made by membranes expressing seHAS.

HA synthesis mediated by the streptococcal HASs occurs at the reducing end, and growing HA-UDP chains are assembled in a processive manner. These enzymes are unable to rebind and extend HA chains once they are released. Although good progress has been made in understanding HAS topology and the mechanism of HA chain elongation, several key questions about HAS function remain unanswered. One question is how HAS controls HA product size. A second is the mechanism by which the HA products are transferred to the cell exterior, since the active sites of the enzyme are intracellular. Others have suggested that HAS makes HA inside the cell and that HA-specific ABC transporters then transport HA to the outside of the cell.

It is believed that membrane-bound HASs are inherently able to translocate HA chains across the bilayer during biosynthesis. This hypothesis is based on a variety of findings, including that the UDP-sugar binding sites are located at or very near the membrane-HAS interface and, unlike virtually all other glycosyltransferases, the HASs are phospholipid-dependent and have multiple trans-membrane domains. In addition, the genetic and biochemical data show that if substrates are present, HAS is the only protein required for HA biosynthesis. When bacteria that do not normally make HA are transformed with the hasA gene and genes for the synthesis of UDP-sugar precursors, the cells make and, importantly, secrete HA into the medium. For example, no other exogenous genes except hasA are needed for Enterococcus faecalis or Bacillus subtilis cells to accumulate HA in the culture medium.

Based on radiation inactivation analysis of two streptococcal HASs and Xenopus HAS1, the active membrane-bound enzymes contain only a single HAS protein (rather than an oligomer), but the protein is associated with an additional mass of ˜23 kDa. This extra mass could not be identified for the active HAS1 enzyme, but was identified as cardiolipin for the streptococcal enzymes. Thus, an active streptococcal HAS is one protein in complex with about 16 molecules of cardiolipin. When purified in the absence of cardiolipin, the streptococcal HASs show very low activity, but when this phospholipid is restored, enzyme activity increases ˜10-fold. Therefore, all HAS family members might require lipid in order to synthesize HA, but different HASs may require different types of lipids. It is proposed that cardiolipin molecules help the enzymes to create a pore-like passage within the HAS-lipid complex, through which a growing HA chain passes.

If the MDs of HAS form a pore through which HA passes during biosynthesis, then there are likely to be very specific interactions between MDs that stabilize this structure and mediate its cycle of making and breaking interactions with the HA chain as the HA-UDP is alternately translocated or extended. The results show that changing either K48 or E327 can inhibit enzyme activity and alter HA product size and support the possibility that K48 and E327 are binding partners that mediate interaction between MD2 and MD4. This latter conclusion is supported strongly by the rescue of both protein expression and enzyme activity in the double-switch mutant, compared to the E327K mutant. Although other explanations can be proposed for rescue of the E327K variant by introducing the K48E change, the simplest is that the E and K residues at these two positions directly interact. Based on the topology of seHAS, K48 and E327 are within the same region of the bilayer (FIG. 14B), and could interact physically. Due to the very short extracellular loops of HAS, MD1-MD2 and MD4-MD5 are likely to be adjacent MD pairs (e.g., FIG. 14C).

It is also possible that the two polar residues within MD2 and MD4 interact with polar groups in HA and are involved in the HA alignment or translocation process during synthesis. When the enzyme is not engaged in HA synthesis, then the two side chains might directly interact as suggested above. An interesting hybrid of these two ideas is that K48 and E327 could switch between these two states in an alternating way (i.e., interacting with each other versus with HA) during chain elongation and translocation. Translocation of HA through the enzyme would likely require the coordinated movement of one or more MDs bound to the chain in order to achieve HA movement. This would have to be a cyclic process in which the interactions between residues in HAS and specific groups within the HA chain were made and then broken as the chain was translocated in a ratchet-like manner through the membrane to the cell exterior.

Although changing either residue influences HA product size, E327 appears to be more critical for seHAS activity than K48. Changing E327 to the very similar Asp had minimal effect on protein expression and reduced HA product size by only ˜12%, whereas changing E327 to Lys destabilized the protein, reduced expression, and drastically inhibited activity. The K48R and K48E mutants were both well expressed, indicating that these changes did not destabilize the protein. The slower migration of seHAS(K48E) in SDS-PAGE and its smaller HA product size (˜19% of wildtype) are consistent with the mutant protein being less compact and hindered in either HA bond formation, HA trans-location or both. All of these results are interpreted to indicate that E327 participates in H-bonding or ionic interactions that are structurally important. The relative tolerance to changes at K48 suggests that if this residue interacts with E327, then E327 likely also has important interactions with other residues. The two side chains could interact either directly or indirectly through water, phospholipid head groups, or other side chains.

The results in the present study indicate that there is not a simple correlation between the affects of a mutation on enzyme activity compared to enzyme stability, as assessed by migration in SDS-PAGE. A possible explanation for this is that both K48 and E327 also likely interact with other residues in HAS and possibly with groups in HA, as noted above. If these two residues interact in a complex, alternating cyclic manner, with side chains in the protein and in HA, then correlations among enzyme activity, protein stability and domain flexibility may not be apparent for individual mutants or may be too complex to sort out without substantially more structural information.

A pore region within HAS would have multiple contacts with the growing HA-UDP chain. Differences in the energetics of these intermolecular contacts among the three eukaryotic HAS isozymes could alter the balance between HA-retention and HA-release forces and result in inherent differences in HA size distributions made by HAS variants or native HAS isozymes (44). For example, a mutation that decreased HA retention by HAS could result in decreased average HA product size. Further studies are needed to test this model and to elucidate the roles of K48, E327, and other conserved amino acids in the ability of HAS to make HA of a particular size.

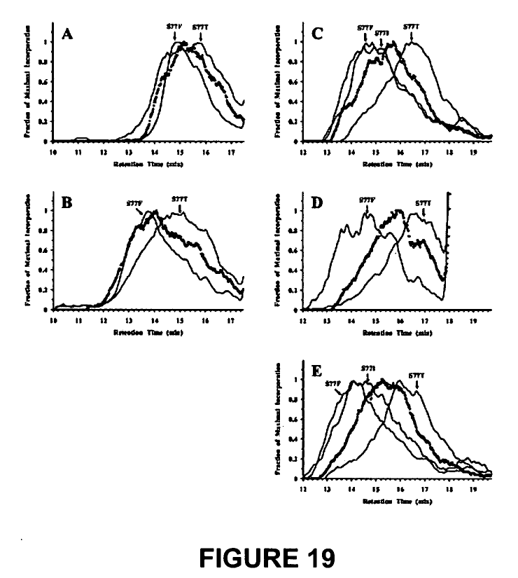

Mutations of a vertebrate HAS also affect HA size distribution. As reported by the inventors in Pummill et al., 2003 (JBC, 278:19808-19814, the contents of which are hereby expressly incorporated herein by reference), site-directed mutagenesis was utilized to examine the differences in size distribution of HA produced by various mutants of the vertebrate HAS enzyme from Xenopus laevis, referred to as xlHAS1. It was discovered that certain xlHAS1 mutants with substitutions for the serine at position 77 synthesize HA products with different size distribution, and of the Ser77 mutants created, S77F and S77I created a larger HA product while S77T created a smaller HA product. On the other hand, S77A, S77D, S77V, S77Y/R271G, and Y107F all made HA with an average size similar to that of wild type xlHAS1. As can be seen in FIG. 19, the average size of HA produced by S77T starts to fall behind that of wild type after 5 min. The average size of HA produced by S77T is smaller than that of wild type even after incubation for 60 min. S77F and S77I make HA with a larger average product size than wild type for all time points after 5 min.

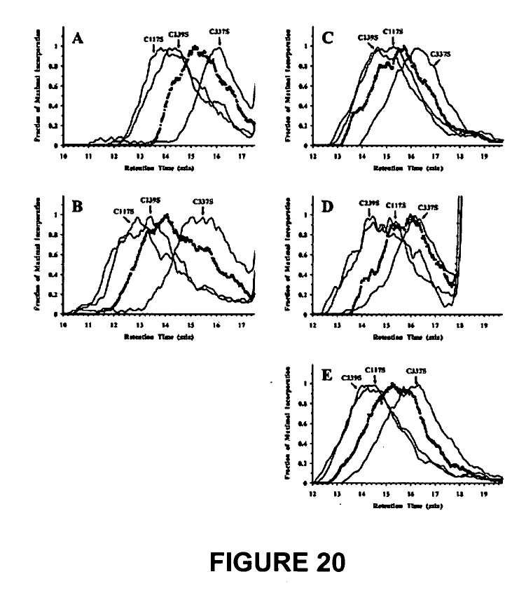

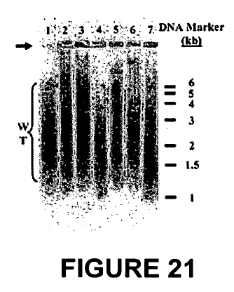

Several cysteine to serine mutants were also tested for changes in the size of the HA product. As shown in FIG. 20, C117S and C239S consistently made larger HA than that of wild type, whereas C337S made HA that was consistently smaller. The results obtained from the gel filtration experiments shown in FIGS. 19 and 20 were confirmed by electrophoretic analysis of the HA produced by wild type and mutant enzymes. The average HA product size was larger than that of wild type for C117S, C239S, S77F, and S77I but smaller for C337S and S77T (FIG. 21).

Alteration of Hyaluronan Product Size by Expression of Synthases with Multiple Mutations. It has been demonstrated herein that the size of HA produced by cells expressing a HAS gene can be altered by mutating the HAS gene at single amino acid residues. It has also been demonstrated herein that by mutating a single amino acid residue of xlHAS1, namely S77, the size of the HA produced by the HAS could be increased or decreased, depending on the amino acid substitution. An alignment of the amino acid sequences of xlHAS1 and seHAS indicate the presence of a region in seHAS similar to that of the S77 region in xlHAS1. Primers were designed that would mutate the amino acid residues in this region of seHAS, ⁵¹LSF⁵³, and the effect on the size of HA produced by cells expressing these mutants was investigated.

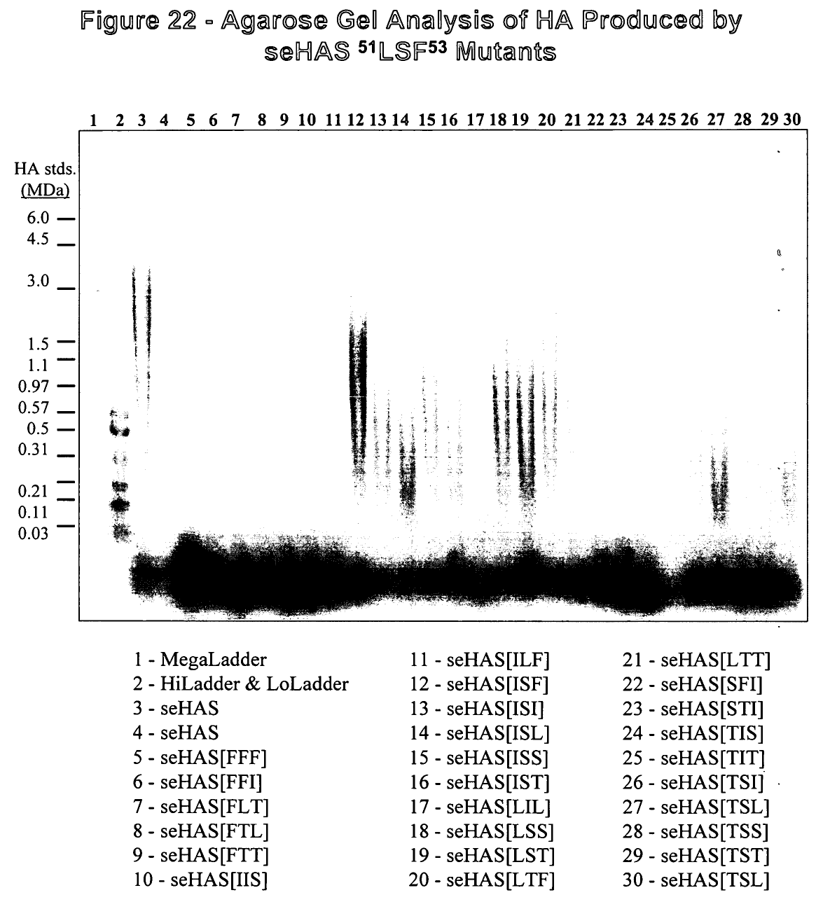

The results of the agarose gel electrophoretic analysis of the HA produced by several of the seHAS enzymes with mutations in the ⁵¹LSF⁵³ region are shown in FIG. 22. The results are also summarized in Table XIII. As can be seen, the amount and size of HA produced by these enzymes when expressed in recombinant host cells can be altered by mutating one or more of the amino acid residues in this region. In all cases save one, the mutation of all three amino acid residues led to the inability to observe any HA production by the synthase. Several mutants, including the single mutants seHAS[F53S] and seHAS[F53T], produce HA that is less than half the size of the HA that is produced by wild-type seHAS (Table XIV).

TABLE XIII
⁵¹LSF⁵³ Mutants

| Mutation | | | Effect on HA (compared to RB227[seHAS]) | | |
|---|---|---|---|---|---|
| 51 | 52 | 53 | Production | Size | SEQ ID NO: |
| F | F | f | none detected | not applicable | 99 |
| F | F | l | none detected | not applicable | 100 |
| F | L | T | none detected | not applicable | 101 |
| F | T | L | low | much smaller | 102 |
| F | T | T | none detected | not applicable | 103 |
| l | l | S | none detected | not applicable | 104 |
| l | L | f | none detected | not applicable | 105 |
| l | s | f | similar | slightly smaller | 106 |
| l | s | l | moderate | much smaller | 107 |
| l | s | L | moderate | much smaller | 108 |
| l | s | S | moderate | smaller | 109 |
| l | s | T | moderate | much smaller | 110 |
| l | l | L | low | much smaller | 111 |
| l | s | S | moderate | smaller | 112 |
| l | s | T | moderate | smaller | 113 |
| l | T | f | moderate | smaller | 114 |
| l | T | T | moderate | smaller | 115 |
| S | F | l | none detected | not applicable | 116 |
| S | T | l | none detected | not applicable | 117 |
| T | l | S | none detected | not applicable | 118 |
| T | l | T | none detected | not applicable | 119 |
| T | s | l | moderate | smaller | 120 |
| T | s | L | moderate | much smaller | 121 |
| T | s | S | moderate | smaller | 122 |
| T | s | T | moderate | smaller | 123 |

The letters in the column under each position indicate the amino acid present at that location.
A capital letter indicates a change from the residue present in the wild-type seHAS.

TABLE XIV
GPC-MALLS Results with ⁵¹LSF⁵³ Mutants.
Clarified media from the samples used for FIG. 22 were
diluted to ˜25 mg/ml HA based on the agarose gel results.
200 μl was injected onto the GPC columns, and the eluate
was monitored for HA molar mass by MALLS. The midpoint
of the refractive index peak for the eluate is listed as the
retention volume.

| | | Mw |
|---|---|---|
| HAS | Retention Volume (ml) | Molar Mass (g/mol) |
| seHAS | 14.2 | 2.34 × 10⁶ |
| seHAS[ISL] | 16.3 | 9.46 × 10⁵ |
| seHAS[IST] | 16 | 1.01 × 10⁶ |
| seHAS[LSS] | 15.3 | 1.07 × 10⁶ |
| seHAS[LST] | 15.4 | 9.07 × 10⁵ |

TABLE XV
SeHAS mutants with multiple mutations.

| | SEQ ID |
|---|---|
| Mutant | NO: |
| seHAS[F349C] | 124 |
| seHAS[F349D] | 125 |
| seHAS[F349Y] | 126 |
| seHAS[F349G] | 127 |
| seHAS[K31T] | 128 |
| seHAS[K31T, F349G] | 129 |
| seHAS[K31S] | 130 |
| seHAS[K31S, F349G] | 131 |
| seHAS[K31N] | 132 |
| seHAS[K31N, F349G] | 133 |
| seHAS[K31Y] | 134 |
| seHAS[T268V] | 135 |
| seHAS[T268V, F349G] | 136 |
| seHAS[T268I] | 137 |
| seHAS[T268I, 349G] | 138 |
| seHAS[T268L] | 139 |
| seHAS[T268L, F349G] | 140 |
| seHAS[N317I] | 141 |
| seHAS[N317I, F349G] | 142 |
| seHAS[N317K] | 143 |
| seHAS[N317K, F349G] | 144 |
| seHAS[N317T] | 145 |
| seHAS[N317R] | 146 |
| seHAS[G344N] | 147 |
| seHAS[G344D] | 148 |
| seHAS[G344E] | 149 |
| seHAS[G344K] | 150 |
| seHAS[T268I, N317I, F349G] | 151 |
| seHAS[T268I, N317T, F349G] | 152 |
| seHAS[T268I, N317R, F349G] | 153 |
| seHAS[T268I, trunc. @ 342) | 154 |
| seHAS[T268I, G344K, F349G] | 155 |
| seHAS[T268I, G344E, F349G] | 156 |
| seHAS[T268I, N317K, F349G] | 157 |
| seHAS[T268I, N317K, G344K, | 158 |
| F349G] | |
| seHAS[T268I, N317R, G344K, | 159 |
| F349G] | |
| seHAS[T268I, N317T, G344K, | 160 |
| F349G] | |

TABLE XVI
GPC-MALLS Results for HAS with Multiple Mutation Sites.
Clarified media from the samples used for FIG. 23 were
diluted to ˜25 μg/ml HA based on the agarose gel results.
200 μl was injected onto the GPC columns, and the eluate was
monitored for HA molar mass by MALLS. The midpoint of the
refractive index peak for the eluate is listed as the
retention volume.

| | Retention | | Retention | |
|---|---|---|---|---|
| | Volume | Mw Molar | Volume | Mw Molar |
| HAS | (ml) | Mass (g/mol) | (ml) | Mass (g/mol) |
| | Experiment 1 | | Experiment 2 | |
| seHAS | 14.2 | 2.34 × 10⁶ | 14.1 | 2.49 × 10⁶ |
| seHAS[F349G]-1 | 13.9 | 3.13 × 10⁶ | 13.8 | 3.15 × 10⁶ |
| seHAS[K31T]-1 | 14.1 | 2.08 × 10⁶ | 14.1 | 2.57 × 10⁶ |
| seHAS[K31T, F349G]-1 | | | 13.8 | 3.20 × 10⁶ |
| seHAS[K31S]-3 | | | 14.2 | 2.45 × 10⁶ |
| seHAS[K31S, F349G]-2 | | | 13.8 | 3.45 × 10⁶ |
| seHAS[K31N]-1 | | | 14.2 | 2.18 × 10⁶ |
| seHAS[K31N, F349G]-2 | | | 13.8 | 2.67 × 10⁶ |
| seHAS[T268V]-1 | 13.9 | 2.56 × 10⁶ | 13.8 | 2.22 × 10⁶ |
| seHAS[T268V, F349G]-3 | | | 13.7 | 3.11 × 10⁶ |
| seHAS[T268I]-1 | 13.8 | 2.55 × 10⁶ | 13.8 | 3.55 × 10⁶ |
| seHAS[T268I, F349G]-1 | | | 13.6 | 3.74 × 10⁶ |
| seHAS[T268L]-1 | 14.4 | 2.18 × 10⁶ | 14.5 | 1.8 × 10⁶ |
| seHAS[N317I]-1 | 14.4 | 1.91 × 10⁶ | 14.2 | 2.66 × 10⁶ |
| seHAS[N317I, F349G]-1 | | | 14 | 3.44 × 10⁶ |
| seHAS[N317K] | | | 13.9 | 3.5 × 10⁶ |
| seHAS[N317K, F349G]-3 | | | 13.8 | 3.46 × 10⁶ |
| seHAS[K31Y]-2 | 14.2 | 2.55 × 10⁶ | 14.2 | 2.79 × 10⁶ |
| seHAS[F349C]-1 | 14.3 | 2.31 × 10⁶ | 14.3 | 1.83 × 10⁶ |
| seHAS[F349D]-2 | 14.8 | 1.57 × 10⁶ | 15 | 2.03 × 10⁶ |
| seHAS[F349Y]-2 | 14.7 | 1.15 × 10⁶ | 14.6 | 1.89 × 10⁶ |
| seHAS[N317T]-1 | | | 14 | 2.94 × 10⁶ |
| seHAS[N317R, R353G]-2 | 14.4 | 1.6 × 10⁶ | | |
| seHAS[N317R]-2 | 14 | 2.92 × 10⁶ | 13.9 | 3.22 × 10⁶ |
| seHAS[G344N]-3 | | | 14.3 | 2.32 × 10⁶ |
| seHAS[G344D]-2 | | | 14 | 3.18 × 10⁶ |
| seHAS[G344E]-2 | 13.9 | 2.58 × 10⁶ | 13.8 | 3.62 × 10⁶ |
| seHAS[G344K]-1 | 13.7 | 3.27 × 10⁶ | 13.9 | 3.04 × 10⁶ |
| | Experiment 3 | | Experiment 4 | |
| seHAS-1 | 14.2 | 2.1 × 10⁶ | 14.1 | 2.16 × 10⁶ |
| seHAS-2 | | | 14.2 | 2.4 × 10⁶ |
| seHAS[F349G]-1 | | | 14 | 2.71 × 10⁶ |
| seHAS[F349G]-3 | | | 14.1 | 2.31 × 10⁶ |
| seHAS[T268I]-1 | | | 14.1 | 2.57 × 10⁶ |
| seHAS[T268I]-3 | | | 14 | 3.05 × 10⁶ |
| seHAS[T268I, F349G]-1 | 13.7 | 3.02 × 10⁶ | 13.8 | 3.14 × 10⁶ |
| seHAS[T268I, F349G]-3 | 13.5 | 3.37 × 10⁶ | 13.8 | 2.95 × 10⁶ |
| seHAS[T268I, N317R, F349G]-1 | 13.7 | 3.48 × 10⁶ | | |
| seHAS[T268I, N317R, F349G]-3 | 13.9 | 2.96 × 10⁶ | | |
| seHAS[G344K]-1 | | | 13.8 | 3.06 × 10⁶ |
| seHAS[G344K]-2 | | | 13.8 | 3.07 × 10⁶ |
| seHAS[T268I, G344K, F349G]-1 | 13.8 | 3.44 × 10⁶ | 14 | 2.67 × 10⁶ |
| seHAS[T268I, G344K, F349G]-3 | 13.8 | 3.33 × 10⁶ | 13.7 | 3.44 × 10⁶ |
| seHAS[T268I, G344E, F349G]-1 | 13.9 | 3.11 × 10⁶ | | |
| seHAS[T268I, N317K, G344K, F349G]-1 | | | 13.8 | 3.64 × 10⁶ |
| seHAS[T268I, N317K, G344K, F349G]-2 | | | 14 | 2.68 × 10⁶ |
| seHAS[T268I, N317R, G344K, F349G]-1 | | | 13.7 | 3.22 × 10⁶ |
| seHAS[T268I, N317R, G344K, F349G]-2 | | | 13.9 | 3.46 × 10⁶ |

In addition, two other HASs with single amino acid mutations (N317K and G344D) and one with three amino acid mutations (K31T, T268I, F349C) have been found to produce 50-90% larger HA than that produced by wild-type seHAS, when the HASs were expressed in B. subtilis RB227 cells grown overnight at 37 C in minimal medium. Therefore, it was determined if the HA product size could be increased to a further extent by expressing HASs with various combinations of these amino acid mutations (see Table XV).

The results of the agarose gel electrophoretic analysis of several of the single mutants are shown in FIG. 23A. It is clear that by altering one amino acid residue in the HAS, the resulting HA products made by recombinant host cells can be effected in both amount and size. The effect can vary at each position depending on which substituting amino acid is used. The ability to alter the size of the HA product is supported by the MALLS data shown in Table XVI, Experiment 1. Based on the single mutation data, seHAS[F349G] was used as a template for the second round of site-directed mutagenesis to obtain the desired double mutants. The results for the double mutants are shown in FIG. 23B and Table XVI, Experiment 2. Again, based on the results from the agarose gel electrophoresis and MALLS analysis, seHAS[T268I, F349G] was chosen as the template for the production of the triple mutants. The data for the triple mutants can be seen in FIG. 23C and Table XVI, Experiment 3. Finally, seHAS[T268I, G344K, F349G] was chosen as the template for the production of quadruple mutants, and the data for these mutants is shown in FIG. 23D. In order to provide a summary of the data, an agarose gel electrophoresis experiment, along with MALLS analysis, was performed on a selection of the mutants. The results for this summary experiment are shown in FIG. 23E and Table XVI, Experiment 4.

As described above for the ⁵¹LSF⁵³ mutants, it can be clearly seen that by altering a single amino acid residue, the size of the HA product can be effected. Altering two different amino acid residues had various effects on the HA product size. For the K31 mutants, the addition of the F349G mutation brought the HA product size up close to that of seHAS[F349G] by itself. SeHAS[T268L] by itself made smaller HA than wild-type seHAS, but when it was combined with F349G, almost all HAS activity was lost. SeHAS with a T268I change alone made larger HA than wild-type seHAS, and when this mutation was combined with F349G even larger HA was produced.

It has been previously shown by the inventors that the HA product size for a given HAS can be altered by mutating amino acid residues in the HAS. However, these experiments were done in vitro and usually dealt with single amino acid mutations. It has been demonstrated herein that the HA product size in vivo can be increased or decreased by a single amino acid mutation. Also, by combining different mutations, the effect can be even greater. The size of HA obtained from the T268I/F349G seHAS mutant (3.74 MDa) is larger than that obtained from any of the single mutants. In the last experiment, the size of HA produced by seHAS[T268I, N31 7K, G344K, F349G] (3.64 MDa) is larger than that produced by any other mutant during that experiment.

### Materials and Methods

Vectors, Primers, and Reagents. pEx-1 was purchased from Promega as part of the Altered Sites Mutagenesis kit. The expression vector pKK223 was from Pharmacis Biotech Inc. E. coli SURE cells were from Stratagene. Successful mutagenesis was achieved with the QUICK CHANGE™ Mutagenesis kit from Stratagene. Primers were synthesized by The Great American Gene Company (Ransom Hill Bioscience, Inc., CA), NBI, or Midland Certified Reagent Company. All of the mutagenic oligonucleotides were synthesized by Genosys Biotechnologies, Inc. (Spring, Tex.) and were purified by reverse-phase chromatography. Cy-5 fluorescent sequencing primers were synthesized by the Molecular Biology Resource Facility, Oklahoma University Health Sciences Center. Nonradiolabeled UDP-GlcUA, and 2,4,6-trihydroxyacetophenone were from Fluka. UDP-GlcNAc was from Sigma. UDP-[¹⁴C]GlcUA (300 mCi/mmol) and ¹⁴C-NEM (40 mCi/mmol) were from New England Nuclear. Agarose was from Bio-Rad. (+)-Biotinyl-3-maleimidopropionamidyl-3,6-dioxaoctanediamine (biotin-PEO-maleimide) was from Pierce Chemical Co. NEM and all other reagents were from Sigma unless otherwise noted. To confirm the entire ORF of HAS mutants, DNA sequencing was performed either using the T7 or PCR sequencing kits from Amersham, or by the micro-sequencing facility operated by the Department of Microbiology & Immunology at the University of Oklahoma Health Sciences Center. Anti-His₅ monoclonal antibody and Ni⁺²-NTA resin were from Qiagen.

Site-Directed Mutagenesis. The seHAS gene with a fusion at the 3′ end encoding a His₆ tail (seHAs-His₆) was cloned into pKK233. Mutagenic primers were designed to change the cysteines to either Ala or Ser at positions 226, 262, 281 and 367. Two complementary oligonucleotide primers encoding the desired mutation were used to create the single Cys mutations (Table XVII). Mutagenesis was carried out using the Quick Change method according to the manufacturer's instructions. The pKK233 plasmid containing the seHAS-His₆ gene was grown in SURE cells, purified using a Spin Miniprep Kit (Qiagen) and analyzed by agarose gel electrophoresis to verify the correct size. The purified pDNA was used as the template for the primer extension reaction with a pair of mutagenic primers. The PCR Amplification conditions for PCR, using pfu DNA polymerase, were 16 cycles of the following: 95° C. for 1 min, 58° C. for 1 min, and 68° C. for 18 min. This amplification generated mutated plasmids with staggered nicks, which was then treated with DpnI to digest the methylated and hemi-methylated parental DNA. The digested pDNA was transformed into SURE cells and colonies were screened for the desired mutations by sequencing the isolated plasmid DNA using fluorescently labeled terminators (ABI Prism 377 MODEL program, v2.1.1). The complete ORFs of selected mutants were confirmed by sequencing in both directions with Cy-5 labeled vector primers on a Pharmacia ALF Express DNA Sequencer. Data were analyzed using ALF Manager, v3.02. The double, triple and null Cys-mutants of seHAS-His₆ were made using the appropriate single, double or triple Cys-mutant plasmid DNA as the template, respectively.

TABLE XVII

Synthetic oligonucleotides used to make
seHAS mutants
The boldface font indicates the altered
codon. All primers shown are in the
sense orientation.

SEQ

| | |
|---|---|
| seHAS | ID |

| | | |
|---|---|---|
| mutant | Sequence | NO: |

| | | |
|---|---|---|
| C226A | 5′-GGTAATATCCTTGTTGCCTCAGGTCCGCTTAGC | 161 |
| C226S | 5′-GGTAATATCCTTGTTTCCTCAGGTCCGCTTAGC | 162 |
| C262A | 5′-ATTGGTGATGACAGGGCCTTGACCAACTATGCA | 163 |
| C226S | 5′-ATTGGTGATGACAGGTCCTTGACCAACTATGCA | 164 |
| C281A | 5′-CAATCCACTGCTAAAGCTATTACAGATGTTCCT | 165 |
| C281S | 5′-CAATCCACTGCTAAATCTATTACAGATGTTCCT | 166 |
| C367A | 5′-TTCATTGTTGCCCTGGCTCGGAACATTCATTAC | 167 |
| C367S | 5′-TTCATTGTTGCCCTGTCTCGGAACATTCATTAC | 168 |

TABLE XVIII

Oligonucleotides for Cys-to-Ser/Ala site
directed mutagenesis of spHAS. Cys
residues within spHAS at positions 124,
225, 261, 280, 366 and 402 were converted
either to Ser using a single mutagenic
oligonucleotide, complementary to the
coding strand, and the Altered Sites kit
or to Ala using a pair of complementary
mutagenic oligonucleotides and the Quick
Change Mutagenesis kit (in the latter
case only the forward primers
complementary to the coding strand are
shown). The altered codons are indicated
in boldface.

SEQ

| | | |
|---|---|---|
| SpHAS | Mutagenic | ID |

| | | |
|---|---|---|
| mutation | Oligonucleotide | NO: |

| | | |
|---|---|---|
| (C124S) | 5′-TAACGTTTCGAGAAATATCCAC | 169 |
| (C225S) | 5′-GGTCCTGAGGAAACTAAAAT | 170 |
| (C261S) | 5′-ATTTGTTAAAGATCGATCATC | 171 |
| (C280S) | 5′-ATCAGTATCAGATCTAGCTGT | 172 |
| (C366S) | 5′-AACATTACGAGATAAAGCAAC | 173 |
| (C402S) | 5′-TTTAATGGTGGATAAAGAATA | 174 |
| (C124A) | 5′-AACGATAACGTTTCGAGCAATATCCACTTCTCT | 175 |
| (C225A) | 5′-CAATGGTCCTGAGGCAACTAAAATATTACC | 176 |
| (C261A) | 5′-AGCATAATTTGTTAAAGCTCGATCATCCCCAAT | 177 |
| (C280A) | 5′-AGGTACATCAGTATCAGCTCTAGCTGTTGATTG | 178 |
| (C366A) | 5′-AACATTACGAGCTAAAGCAAC | 179 |
| (C402A) | 5′-CGTATTTTTAATGGTGGCTAAAGAATAAAGTTT | 180 |

Single mutants of spHAS were generated by the Altered Sites Mutagenesis or Quick Change Mutagenesis protocols using primers (Table XVIII) designed to change the Cys residues at positions 124, 225, 261, 280, 366, or 402 of spHAS containing His₆ at the C-terminus (Tlapak-Simmons, et al., 1999a). After generating and confirming the entire sequence of each spHAS(Cys-to-Ser) mutant produced in the Altered Sites vector, internal restriction sites within the HAS ORF were used to transfer mutated regions to the spHAS insert in pKK223 (this vector carrying HAS is designated pKK3K). Cys-to-Ala mutants of spHAS were generated directly in the pKK3K vector using the Quick Change mutagenesis method. Site directed mutagenesis was used to generate the C124, C402A double mutant, and then C366A was added by restriction fragment exchange to generate a triple mutant. Site directed mutagenesis was also used to create the double mutant spHAS(C261A,C280A). The mutants containing five or six mutated Cys residues were generated by utilizing restriction sites to combine fragments of spHAS containing different mutations. For example, AvrII and MfeI were used to combine the spHAS(C124A,C366A,C402A) triple Cys-mutant and the spHAS(C261A,C280A) double mutant to create spHAS with only Cys²²⁵ intact. Finally, BglII and AvrII were used to splice spHAS(C225A) into the latter quintupleCys-mutant to generate the Cys-null clone, designated spHAS^{Cys-null}. All Cys-to-Ala/Ser mutants were confirmed over the full ORF by automated DNA sequencing.

Effect of sulfhydryl reagent treatments on seHAS and spHAS activity and determination of the kinetic constants of seHAS Cys mutants. E. coli SURE cells transformed with plasmids 9containing various seHAS mutants were grown in LB medium with vigorous shaking at 32° C. to A₆₀₀ ˜0.8 and induced with 1 mM isopropyl-p-thiogalactoside for 3 h. Cells were harvested and membranes were prepared as described previously. The kinetic constants for HAS were determined at 37° C. in 100 μl of 50 mM sodium and potassium phosphate, pH 7.0, with 20 mM MgCl₂, 1 mM DTE, 240 μM UDP-GlcUA, 0.7 μM UDP-[¹⁴C]GlcUA and 0.6-1.0 mM UDP-GlcNAc. in 100 μl of 25 mM sodium and potassium phosphate, pH 7, containing 50 mM NaCl, 20 mM MgCl₂, 1 mM dithiothreitol, 1 mM UDP-GlcUA, 0.68 μM UDP-GlcUA and 1 mM UDP-GlcNAc. Some assays also contained 0.1 mM EDTA and 20% glycerol (v/v). To initiate the enzyme reaction, ˜0.5-40 μg of membrane protein was added and the mixtures were gently shaken in a MicroMixer X-36 (Tiatec) at 30° C. for 1-2 h. Reactions were terminated by the addition of SDS to a final concentration of 2% (w/v). The incorporation of radioactive [¹⁴C]GlcUA was determined by descending paper chromatography and the Kₘ and Vₘₐₓ values were determined as described by Tlapak-Simmons et al (1999b). Data were analyzed by the methods of Michaelis-Menton or Hill. Protein content was determined by the method of Bradford using bovine serum albumin as the standard. All Cys-mutant or sulfhydryl-treated seHAS samples were assayed in duplicate or triplicate using two or three independent membrane preparations. Results are presented as the mean ± standard errors. All enzyme assays were performed under conditions that were linear with respect to time and protein concentration. None of the seHAS variant enzymes were unstable under the conditions employed.

Determination of HA size produced by seHAS variants. The relative Mᵣ of the HA synthesized by wild-type seHAS or the Cys-mutants was determined by agarose gel electrophoresis of ¹⁴C-labeled HA products synthesized under the assay conditions described above. The wild-type seHAS synthesizes and releases an HA chain in <5 min under these steady-state conditions, so that each enzyme molecule on average synthesizes >10 HA chains during the incubation. Reactions were terminated by heating at 95° C. for 1 min, the mixtures were then centrifuged at high speed and the HA-containing supernatants were recovered. The samples were concentrated ˜10-fold using Microcon YM-3 filters (Amicon Bioseparations, Inc.) and treated with DNase and RNase (4 μg/ml each) in the presence of 60 mM MgCl₂ for 30 min at 22° C. The samples and a combination of DNA standards were then electrophoresed on a 1.3% (w/v) agarose gel at 80-90 V. The gels were dried without heating and exposed to Biomax-MR film (Kodak) for 1-4 weeks. The autoradiograms were scanned to create digital files using a Fluorchem™8000 (Alpha Innotech Corp.) image analysis station. As a control, samples were treated with Streptomyces hyaluronate lyase (80 units) at 37° C. overnight, which resulted in the complete loss of radiolabeled bands.

Determination of HA Chain Length Produced by spHAS Variants (Table X). HAS mutant HA size distribution was determined by agarose gel electrophoresis. Membranes containing wildtype or mutant Has were incubated in 0.5 mM UDP-GlcUA and 1.5 mM UDP-GlcNAc in 25 mM sodium/potassium phosphate, pH 7.0, 75 mM NaCl, 1 mM DTT, 15% glycerol, 10 mM MgCl₂, with 0.68-8.16 μM UDP-[¹⁴C]GlcUA for one hour at 30 C, while shaking in a Taitec E-36 micromixer. EDTA was added to 0.1 M to stop the reactions. 1 mg/ml ovine testicular hyaluronidase was added to control tubes and incubated 1 hour at 37 C to ensure the product formed was HA. 5 mg/ml Pronase was added to digest protein components that may bind HA, and the reactions were incubated >12 hours at 4 C. Reactions were either loaded directly to 1% agarose gels or were ethanol precipitated prior to loading. Components of the reaction were retained in or near the wells after electrophoresis. Ethanol precipitation was done to reduce this contamination. Reactions were centrifuged at 21,000×g for 10 min. One-tenth the volume of 3 M sodium acetate, pH 5.2, and three volumes of ethanol were added to supematants. The tubes were mixed and incubated at −20 C for 1 hour. The HA was collected by centrifugation at 21,000×g for 10 min. The pellet was air dried and suspended in either water or PBS. The suspension was loaded to agarose gels along with High Molecular Weigh and KB ladder DNA standards from Gibco BRL. After electrophoresis was stopped, the gel was dried and exposed to a phosphoscreen for 1 hour to >2 weeks. The gel image was obtained from the screen using a Molecular Dynamics Phosphoimager. HA chain size was estimated by migration distance relative to DNA standards. HA product size distribution was estimated by integration over MW ranges of the integrated density value (IDV) curve obtained for each lane. The preferred HA product size (peak size) was designated by the MW range in which the greatest IDV was located.

Determination of seHAS protein concentration in membranes and normalization of seHAS activity. The recombinant seHAS protein in isolated membranes is a major component comprising ˜5-8% of the total protein, is well separated from other major proteins by SDS-PAGE and can be readily identified in Coomassie Blue-stained gels. E. coli membranes containing wild-type or mutant seHASs were solubilized and electrophoresed on 10% (w/v) gels following the procedure of Laemmli for SDS-PAGE. The amount of seHAS protein in each membrane preparation was quantitated by image analysis of the stained gel using a Fluorchem™8000 (Alpha Innotech Corp). The linearity of Coomassie Blue-stained seHAS bands was verified by loading different amounts of membrane protein. To generate a standard curve, various amounts of affinity purified seHAS-His₆ were subjected to identical SDS-PAGE and the Integrated Density Value (IDV) was determined for each band. The IDVs were plotted against pmol of pure seHAS. The IDV values for seHAS bands in membranes containing wild-type or mutant proteins were then compared with the standard to estimate the seHAS protein content per mg membrane protein. These data were then used to normalize the seHAS enzyme activity in the membrane preparations forwild-type and each variant seHAS.

Chemical modification of HAS in membranes. Stock solutions (10-100 mM) of NEM, IAA or other sulfhydryl reagents were made in PBS, pH 7.0. Suspensions of membranes containing seHAS or spHAS were incubated with 0-5 mM of the sulfhydryl reagent at 4° C. and the reactions were stopped by adding DTE to a final concentration of 10 mM. The membranes were then assayed for HAS activity as described above.

Labeling of seHAS with ¹⁴C-NEM. Isolated membranes from wild-type seHAS and each of the six double Cys-mutants of seHAS were incubated at 4° C. with 2.5 mM ¹⁴C-NEM (˜8×10⁶ dpm) for 5 min. The reactions were terminated by the addition of DTE to a final concentration of 5 mM. Total membrane proteins were precipitated by incubation with 10% trichloroacetic acid overnight at 4° C. and free ¹⁴C-NEM was then removed by two cycles of centrifugation and resuspension with 5% trichloroacetic acid. The precipitated proteins were dissolved in 1×SDS Laemmli sample buffer, neutralized by the addition of 0.1 N NaOH and analyzed by SDS-PAGE using a 10% gel. The Coomassie Blue-stained gel was scanned using a Model PDSIP60 densitometer (Molecular Dynamics), then treated with scintillants and subjected to fluorography using Biomax-MR (Kodak) film and an exposure of approximately one week. E. coli membranes prepared from cells transformed with vector alone, containing no seHAS, were included as a control.

MALDI-TOF analysis of seHAS derivatives. Wild-type seHAS-His₆ was bound to a Ni⁺²-nitrilotriacetic acid chelate resin (Qiagen), washed and treated with biotin-PEO-maleimide (10 mg/ml) for 2 h on ice. After washing the column, the enzyme was eluted with distilled water containing 0.5% (v/v) trifluoroacetic acid and 0.02% (w/v) dodecylmaltoside. The degree of modification of Cys residues in treated seHAS samples was determined using a MALDI-TOF Voyager Elite mass spectrometer (Applied Biosystems, Framingham, Mass.), which was equipped with a N₂ laser (337 nm), located in the NSF EPSCoR Oklahoma Laser Mass Spectrometry Facility. The sample (1 μl) was spotted to a sample plate, followed by matrix solution (1 μl) and allowed to air dry. The matrix used was a 20 mg/ml solution of 2,4,6-trihydroxyacetophenone in 50% acetonitrile containing 0.1% trifluoroacetic acid and 0.05% (w/v) dodecylmaltoside. Samples were analyzed in the linear, positive ion mode using a delayed extraction of 300 ns, a grid voltage of 87.8%, and were subject to a 25 kV accelerating voltage. External and internal calibrations were routinely performed using horse apomyoglobin and bovine serum albumin (16,951 and 66,430 Da, respectively). Spectra were an average of 80-120 scans and were processed using the 19 point Savitsky-Golay smoothing option included in the software provided by the manufacturer.

Determination of recombinant spHAS-H₆ content in membranes. Membranes were isolated from E. coli SURE strains expressing mutant or wildtype spHAS, fractionated by SDS-PAGE according to the procedure of Laemmli (1970) and the proteins were electrotransferred to nitrocellulose as described by Towbin et al. (1979) with minor modifications (Tlapak-Simmons, et al., 1998). Western analysis was performed by incubating these blots for 1 h at room temperature with a biotinylated anti-His₅ monoclonal antibody, as the primary antibody, then washing and incubating with 3 μg/ml ¹²⁵I-streptavidin, prepared as described hereinabove. After 1 h at room temperature, the blots were washed, dried and exposed to a Phosphoscreen for 1 to 72 h. The screens were analyzed using a Molecular Dynamics Phosphoimager and integrated density values were obtained for each spHAS band. Integrated density values were also obtained for increasing amounts of affinity-purified spHAS (Tlapak-Simmons et al., 1999a), which was used as an internal standard in the same blot to generate a standard curve. Dilutions of membrane samples were made as necessary to ensure that all estimates of HAS quantity were in the linear range of the assay. Those membrane samples giving a linear response with increasing protein were then compared to the standard curve to calculate the amount of spHAS present per μl membrane suspension. These values were then used to normalize the kinetic results obtained using membranes from the various spHAS mutants.

Enzymatic Analysis of HAS Mutants. E. coli SURE cells, previously transformed with plasmids containing wildtype or mutant spHAS genes, were grown to an A₆₀₀ of ˜1.2 and induced with 1 mM isopropyl thio-β-D-galactoside for three h. Cells were harvested and membranes were prepared as described previously (Tlapak-Simmons, et al., 1999a). The activities of mutant spHAS variants were assessed by measuring their Vₘₐₓ and Kₘ values in isolated membranes, normalized as described above for the amount of enzyme expressed. The Kₘ values were determined using a descending paper chromatography assay (Tlapak-Simmons, et al., 1999b), holding one UDP-sugar substrate constant and varying the other from 0.01 to 4 mM. Data were analyzed by linear regression using Haynes-Wolf plots for UDP-GlcUA or Hill plots for UDP-GlcNAc.

Inhibition of spHAS activity by NEM. Membrane preparations from wildtype and various spHAS mutants (i.e., C124,402A, C261,280A, C124,366,402A, C124,261,280,366,402A, and the Cys-null mutant) were incubated in 50 mM sodium, potassium phosphate, pH 7.0, 75 mM NaCl and 10% (v/v) glycerol with or without 20 mM NEM for 90 min on ice. The ability of the membrane samples to synthesize HA was then assessed by adding the following to the final concentrations indicated: 1 mM UDP-GlcUA, 1 mM UDP-GlcNAc, 0.68 μM UDP-[¹⁴C]GlcUA in 25 mM sodium/potassium phosphate, pH 7, 75 mM NaCl, 10 mM MgCl₂, 1 mM dithiothreitol, 0.1 mM EGTA, 15% glycerol. Reactions were shaken for 1 h at 30° C. in a Taitec E-36 micromixer, stopped by the addition of SDS to 2% (w/v), and spotted onto No. 3MM Whatman paper for descending paper chromatography overnight using 1 mM ammonium acetate pH 5.5/ethanol (7:13). [¹⁴C]GlcUA incorporation into high molecular weight HA was assessed by liquid scintillation spectroscopy to determine the radioactivity remaining at the origin. Confirmation that the latter material is authentic HA was obtained by showing its complete loss after treatment with streptomyces hyaluronidase.

Assessment ofDisulfide Bond Formation in spHAS by MALDI-TOF MS. Wildtype spHAS-His₆ was bound to a Ni⁺² chelate column (Qiagen) and washed as previously described (Tlapak-Simmons, et al,. 1999a). While still bound to the resin, the enzyme was incubated with biotin-PEO-maleimide (10 mg/ml) in the presence or absence of 6 M guanidinium-HCl for 2 h at 4° C. The column was washed and spHAS-His₆ was eluted with distilled water containing 0.5% (v/v) trifluroacetic acid and 0.02% (w/v) dodecylmaltoside. To assess the degree of modification of Cys residues, samples containing purified spHAS were analyzed by MALDI-TOF mass spectrometry using a Voyager Elite mass spectrometer (Applied Biosystems, Framingham, Mass.), which was equipped with a N₂ laser (337 nm), located in the NSF EPSCoR Oklahoma Laser Mass Spectrometry Facility. A 1 μl aliquot of sample was spotted to a sample plate followed by 1 μl of matrix and allowed to air dry. The matrix used was a saturated solution of 2,4,6-trihydroxyacetophenone in 50% acetonitrile containing 0.1% trifluoroacetic acid and 0.005% (w/v) dodecylmaltoside. Samples were analyzed in the linear, positive ion mode using a delayed extraction of 300 ns, a grid voltage of 87.8%, and were subject to a 25 kV accelerating voltage. External calibrations were performed routinely using horse apomyoglobin and bovine serum albumin (16,951 and 66,430 Da, respectively). Data were routinely processed using the 19 point Savitsky-Golay smoothing option included in the software provided by the manufacturer.

Construction of HAS's with multiple mutations. In order to produce the HAS enzymes with mutations in the ⁵¹LSF⁵³ region, primers were designed for use with the QuikChange II XL mutagenesis kit from Stratagene. As shown in Table XIX, these primers would allow for the conversion of each residue to either F, I, L, S, or T. Primers were also designed which would reproduce the mutations in the variants producing larger HA than wild-type seHAS, as well as give three additional possible mutations per site. The forward primer sequence and possible amino acid mutations for each site are listed in Table XIX.

Mutations were created in a seHAS gene contained on a plasmid used for integrating the HAS into the B. subtilis RB227 chromosomal DNA. The plasmid contains genes for resistance to both ampicillin (amp) and chloramphenicol (chlor), the latter of which is integrated into the B. subtilis chromosomal DNA along with the HAS. Mutagenesis was carried out according to the manufacturer's instructions, with a two minute per kilobase extension time. The mutated plasmid mixture was transformed into E. coli XL10-Gold competent cells from Stratagene, according to the manufacturers instructions. Isolated colonies from LBₐₘₚ agar plates were grown in LBₐₘₚ culture medium, and individual plasmids were obtained using the MiniPrep kit from Qiagen. At the Oklahoma Medical Research Foundation Sequencing Core Facility, sequencing was performed on the plasmids to determine the mutations present in the HAS prior to transformation and integration into the B. subtilis cells. Double, triple, and quadruple mutants were obtained by performing mutagenesis on one of the single, double, or triple mutants, respectively.

Plasmids containing the desired HAS mutant genes were linearized by digestion with the restriction enzyme PstI. Competent B. subtilis RB227 cells (150 μl aliquots) were incubated at 37° C. for one hour in a mixture of LB and Spizizen II media with 2 mM EGTA, 0.2 μg/ml chlor, and ˜0.4 μg of individual linearized plasmid DNA. The mixture was then plated on LB_{chlor} agar plates and individual colonies were propagated in LB_{chlor} media for glycerol stock production.

The B. subtilis cells expressing the various HAS genes were incubated for ˜16 hours at 37° C. in minimal media. The cells were then removed by centrifugation at 3,000×g for 20 minutes followed by filtration of the media through a 0.45 μm hydrophilic PVDF filter. The size of the HA present in the medium was determined by both agarose gel electrophoresis with visualization by Stains-All and by size-exclusion chromatography/multi-angle laser light scattering (SEC-MALLS) analysis. For each sample, 4 or 9 μl of clarified medium was mixed with 4 μl of 2×running dye or 1 μl of 10×running dye, respectively, and electrophoresed on 0.6% agarose gels in 1×TAE. Voltage was increased stepwise from 20 to 60 volts. Gels were then stained overnight with Stains-All and destained with water. SEC-MALLS analysis was employed to determine the absolute molecular masses of HA products. Polymers (˜5 μg mass; 200-μl injection) were separated on two PL Aquagel-OH 60 (15 μm) columns (7.5×300 mm, Polymer Laboratories, Amherst, Mass.) in tandem. The columns were eluted with 50 mM sodium phosphate, 150 mM NaCl, pH 7, at 0.5 ml/min. MALLS analysis of the eluant was performed by a DAWN DSP Laser Photometer in series with an OPTI-LAB DSP interferometric refractometer (632.8 nm; Wyatt Technology, Santa Barbara, Calif.). The ASTRA software package was used to determine the absolute average molecular mass using a dn/dc coefficient of 0.153 determined by Wyatt Technology.

TABLE XIX

Mutagenic Primer Sequences and Amino Acid
Substitutions
The sequences of the forward primers and
the possible amino acid substitutions are
listed.
The codons for the amino acids being
mutated are underlined in the primer
sequences.

SEQ

| | | |
|---|---|---|
| SeHAS | Possible AA | ID |

| | | | |
|---|---|---|---|
| AA | Forward Primers | substitutions | NO: |

| | | | |
|---|---|---|---|
| ⁵¹LSF⁵³ | CCTATTAGTCAAAATGTCCWYWWYWW | F, I, L, S, T | 181 |
| | YWTTTTACAAGCCATTTAAGGG | at each | |
| | | position | |
| K31 | CAATGTTTATCTCTTTGGTGCTWMCG | N, S, T, Y | 182 |
| | GAAGCTTGTCAATTTATGG | | |
| T268 | GGTGCTTGACCAACTATGCANTCGAT | F, I, L, V | 183 |
| | TTAGGAAAGACTGTTTATC | | |
| N317 | CTGTTAAGAAAATCATGAACANACCT | I, K, R, T | 184 |
| | TTTGTAGCCCTATGGACC | | |
| G344 | CTGTGGTGGATTTCTTTGTARASAAT | D, E, K, N | 185 |
| | GTCAGAGAATTTGATTG | | |
| F349 | CTTTGTAGGCAATGTCAGAGAAKRCG | C, D, G, Y | 186 |
| | ATTGGCTCAGGGTTTTAGC | | |

Thus it should be apparent that there has been provided in accordance with the present invention a recombinant host cell having a purified nucleic acid segment having a coding region encoding enzymatically active HAS introduced therein, as well as methods of producing hyaluronic acid from the recombinant host cell, that fully satisfies the objectives and advantages set forth above. Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

### REFERENCES

The following references, to the extent that they provide exemplary procedural or other details supplementary to those set forth herein, are specifically incorporated herein by reference in their entirety as though set forth herein particular.
1. Abatangelo, G., and Weigel, P. H. (2000) (editors) New Frontiers in Medical Sciences:Redefining Hyaluronan. Elsevier Science B. V., Amsterdam.
2. Appel, A., Horwitz, A. L., and Dorfman, A. (1979) J. Biol. Chem. 254, 12199-12203.
3. Atkinson, E. M. and Long, S. R. (1992) Mol. Plant. Microbe. Interact. 5, 439-442.
4. Baggenstoss, B. A. and Weigel, P. H. (2006) Anal. Biochem. 352, 243-251.
5. Bliss, J. M. and Silver, R. P. (1996) Mol. Microbiol. 21, 221-231.
6. Bradford, M. M. (1976) Anal. Biochem. 72, 248-254.
7. Breton, C., and Imberty, A. (1999) Curr. Opin. Struct. Biol. 9, 563-571.
8. Chakraborti, P. K., Garabedian, M. J., Yamamoto, K. R., and Simons, S. S., Jr. (1992) J. Biol. Chem. 267,11366-73.
9. DeAngelis, P. L., Papaconstantinou, J. and Weigel, P. H. (1993a) J. Biol. Chem. 268,14568-14571.
10. DeAngelis, P. L., Papaconstantinou, J. and Weigel, P. H. (1993b) J. Biol. Chem. 268,19181-2 19184.
11. DeAngelis, P. L., Yang, N. and Weigel, P. H. (1994) Biochem. Biophys. Res. Commun. 199, 1-10.
12. DeAngelis, P. L and Weigel, P. H. (1994) Biochem. 33, 9033-9039.
13. DeAngelis, P. L. (1996) Biochem. 35, 9768-9771.
14. DeAngelis, P. L. and Achyuthan, A. M. (1996) J. Biol. Chem. 271, 23657-23660.
15. DeAngelis, P. L., Jing, W., Drake, R. R. and Achyuthan, A. M. (1998) J. Biol. Chem. 273,8454-8458.
16. DeAngelis, P. L., Jing, W., Graves, M. V., Burbank, D. E. and Van Etten, J. L. (1997) Science, 278,1800-1803.
17. DeAngelis, P. L. (1999) Cell. Mol. Life Sci. 56, 670-682.
18. Determan, H. (1968) Gel Chromatography, Springer-Verlag, Inc. New York.
19. Evered, D., and Whelan, J. (eds.) (1989) The biology of hyaluronan. Ciba Fnd. Symposium 143,1-288.
20. Fenderson, B. A., Stamenkovic, I., and Aruffo, A. (1993) Differentiation 54, 85-98.
21. Frillingos, S., Sahin-Toh, M., Wu, J. and Kaback, H. R. (1998) FASEB J. 12, 1281-1299.
22. Heldermon, C. D., DeAngelis, P. L. and Weigel P. H. (2001) J. Biol. Chem. 276, 2037-2046.
23. Heldermon, C. D., Tlapak-Simmons, V. L., Baggenstoss, B. A., and Weigel, P. H. (2001) Glycobiology, 11, 1017-1024.
24. Hill, A. V. (1913) Biochem. J. 7, 471-480.
25. Horton, M. R., Olman, M. A., Bao, C., White, K. E., Choi, A. M., Chin, B. Y., Noble, P. W., and Lowenstein, C. J. (2000) Am. J. Physiol. Lung Cell. Mol. Physiol. 279, 707-715.
26. Itano, N. and Kimata, K. (1996a) J. Biol. Chem. 271, 9875-9878.
27. Itano, N. and Kimata, K. (1996b) Biochem. Biophys. Res. Commun. 222, 816-820.
28. Ishimoto, N., Temin, H. M., and Strominger, J. L. (1966) J. Biol. Chem. 241, 2052-2057.
29. Itano, N., Sawai, T., Yoshida, M., Lenas, P., Yamada, Y., Imagawa, M., Shinomura, T., Hamaguchi, M., Yoshida, Y., Ohnuki, Y., Miyauchi, S., Spicer, A. P., McDonald, J. A., and Kimata, K. (1999) J. Biol. Chem. 274, 25085-25092.
30. Ito, K., Sato, T., and Yura, T. (1977) Cell, 11, 551-559.
31. Jung, K., Jung, H., and Kaback, H. R. (1994) Biochemistry 33, 3980-3985.
32. Kass, E. H. and Seastone, C. V. (1944) J.Exp. Med. 79, 319-330.
33. Kato, H., Tanaka, T., Nishioka, T., Kimura, A., and Oda, J. (1988) J. Biol. Chem. 263, 11646-11651.
34. Knudson, C. B., and Knudson, W. (1993) FASEB J. 7,1233-1241.
35. Kumari, K. and Weigel, P. H. (1997) J. Biol. Chem., 272, 32539-32546.
36. Kumari, K., Tlapak-Simmons, V. L., Baggenstoss, B. A., and Weigel, P. H. (2002) J. Biol. Chem., 277, 13943-13951.
37. Kumari, K., Baggenstoss, B. A., Parker, A. L., and Weigel, P. H. (2006) J. Biol. Chem., 281:11755-11760.
38. Laemmli, U. K. (1970) Nature 227, 680-685.
39. Lansing, M., Lellig, S., Mausolf, A., Martini, I., Crescenzi, F., O'Regan, M., and Prehm, P. (1993) Biochem. J. 289,179-184.
40. Laurent, T. C., and Fraser, J. R. E. (1992) Hyaluronan. FASEB J. 6, 2397-2404.
41. Lee, H. G. and Cowman, M. K. (1994) Anal. Biochem. 219, 278-287.
42. Lin, Y., Mahan, K., Lathrop, W. F., Myles, D. G., and Primakoff, P. (1994) J. Cell Biol. 125, 1157-1163.
43. Malinowski, N. M., Cysyk, R. L., and August, E. M. (1995) Biochem. and Molec. Biol. Intl. 35, 1123-1132.
44. Markovitz, A., J. A. Cifonelli, and Dorfman A. (1959) J. Biol. Chem. 234, 2343-2350.
45. McDonald, J. and Spicer, A. (1998). In Science of Hyaluronan Today (V. C. Hascall and M. Yanagishita, eds) chapter 7, www.GlycoForum.qr.ip.
46. Meyer, M. F. and Kreil, G. (1996) Proc. Natl. Acad. Sci. USA 93, 4543-4547.
47. Michaelis, L. and Menten, M. L. (1913) Biochem. Z. 49, 333-338.
48. Moxon, E. R. and Kroll, J. S. (1990) Curr. Top. Microbiol. Immunol. 6, 65-85. Ng, K. F. and Schwartz, N. B. (1989) J. Biol. Chem. 264, 11776-11783.
49. Ohno, S., Tanimoto, K., Fujimoto, K., Ijuin, C., Honda, K., Tanaka, N., Doi, T., Nakaharra, M., Tanne, K. (2001) Biochim Biophys Acta 1520, 71-78.
50. Ohya, T. and Kaneko, Y. (1970) Biochim. Biophys. Acta 198, 607-609.
51. Petrov, V. V., Pardo, J. P., and Slayman, C. W. (1997) J. Biol. Chem. 272, 1688-1693.
52. Philipson, L. H., and Schwartz, N. B. (1984) J. Biol. Chem. 259, 5017-5023.
53. Prehm, P. (1983) Biochem. J. 211, 181-189.
54. Prehm, P. (1983) Biochem. J. 211, 191-198.
55. Prehm, P. (1984). Biochem. J. 220: 597-600.
56. Prehm, P. and Mausolf, A. 1986. Biochem. J. 235:887-889.
57. Pummill, P. E. and DeAngelis, P. L. (2002) J. Biol.Chem., 277, 21610-21616.
58. Pummill, P. E. and DeAngelis, P. L. (2003) J. Biol. Chem., 278,19808-19814.
59. Pummill, P. E., Kempner, E. S., and DeAngelis, P. L. (2001) J. Biol. Chem., 276,39832-39835.
60. Rosa, F., Sargent, T. D., Rebbert, M. L. Michaels, G. S., Jamrich, M., Grunz, H., Jonas, E., Winkles, J. A. and Dawid, I. B. (1988) Dev. Biol. 129,114-123.
61. Shyjan, A. M., Heldin, P., Butcher, E. C., Yoshino, T. and Briskin, M. J. (1996) J. Biol. Chem. 271, 23395-23399.
62. Spicer, A. P., Augustine, M. L. and McDonald, J. A. (1996) J. Biol. Chem. 271, 23400-23406.
63. Spicer, A. P. and McDonald, J. A. (1998) J. Biol. Chem. 273, 1923-1932.
64. Sugahara, K., Schwartz, B. N., and Dorfman, A. (1979) J. Biol. Chem. 254: 6252-6261.
65. Tamura, N., Konishi, S., Iwaki, S., Kimura-Someya, T., Nada, S., Yamaguchi, A. (2001) J. Biol. Chem. 276, 20330-20339.
66. Tlapak-Simmons V. L., Baggenstoss, B. A., Clyne, T. and Weigel, P. H. (1999a) J. Biol. Chem. 274, 42394245.
67. Tlapak-Simmons V. L., Baggenstoss, B. A., Kumari, K., Heldermon, C., and Weigel, P. H. (1999b) J. Biol. Chem. 274, 4246-4253.
68. Tlapak-Simmons, V. L., Kempner, E. S., Baggenstoss, B. A. and Weigel, P. H. (1998) J. Biol. Chem. 273, 26100-26109.
69. Toole, B. P. (1997) J. Intem. Med. 242, 35-40.
70. Towbin, H., Steahelin, T. and Gordon, J. (1979) Proc. Natl. Acad. Sci. U.S.A. 76, 4350-4354.
71. Triscott, M. X. and van de Rijn, I. (1986) J. Biol. Chem. 261, 6004-6009.
72. Usui, T., Suzuki K., Kaji, Y., Amano, S., Miyata, K., Heldin, P., Yamashita, H. (1999) Invest. Ophthalmol. Vis. Sci. 40, 563-7.
73. van de Rijn, I. and Drake, R. R. (1992) J. Biol. Chem. 267, 24302-24306.
74. Voss, J., Hubbell, W. L., Hemandez-Borrell, J. and Kaback, H. R. (1997) Biochemistry 36, 15061-15066.

Wang, Q. and Kaback, H. R. (1999) Biochemistry 38, 16777-16782.
75. Ward, P. N., Field, T. R., Ditcham, W. G. Maguin, E. and Leigh, J. A. (2001) Infect. Immun. 69, 392-399.
76. Watanabe, K. and Yamaguchi, Y. (1996) J. Biol. Chem. 271, 22945-22948.
77. Weigel, P. H., Hascall, V. C. and Tammi, M. (1997) J. Biol. Chem. 272,13997-14000.
78. Weigel, P. H. (1998). Bacterial hyaluronan synthases. In Science of Hyaluronan Today (V. C. Hascall and M. Yanagishita, eds) chapter 6, www.GlycoForum.gr.ip.
79. Wessels, M. R., Goldberg, J. B., Moses, A. E., and Dicesare, T. J. (1994) Infect. Immun. 62, 433-441.
80. Wessels, M. R., Moses, A. E., Goldberg, J. B., and Dicesare T. J. (1991) Proc. Natl. Acad. Sci. U.S.A. 88, 8317-8321.
81. White, S. H. and Wimley, W. C. (1999) Annu. Rev. Biophys. Biomol. Struct. 28, 319-365.
82. Whitnack, E., Bisno, A. L., and Beachey, E. H. (1981) Infect. Immun. 31, 985-991.
83. Wolfgang, C. L., Zhang, Z-P., Gabriel, J. L., Pieringer, R. A., Soprano, K. J., and Soprano, D. R. (1997) J. Biol. Chem. 272, 746-753.
84. Wu, J., and Kaback, R. H., (1994) Biochem. 33, 12166-12171.
85. Yang, et al., (1994) EMBO J. 13:286-960.
86. Yoshida, M., Itano, N., Yamada, Y. and Kimata, K. (2000) J. Biol. Chem. 275, 497-506.

What is claimed is:

1. A functionally active hyaluronan synthase having an altered enzymatic activity as compared to a corresponding functionally active native hyaluronan synthase, wherein the functionally active hyaluronan synthase has at least one modified amino acid residue therein as compared to the corresponding functionally active native hyaluronan synthase.

2. The functionally active hyaluronan synthase of claim 1, wherein the corresponding functionally active native hyaluronan synthase is selected from the group consisting of spHAS, seHAS, suHAS and pmHAS.

3. The functionally active hyaluronan synthase of claim 2, wherein the corresponding functionally active native hyaluronan synthase has an amino acid sequence essentially as set forth in at least one of SEQ ID NOS:2, 4, 6, 8, 10, 12 and 14.

4. The functionally active hyaluronan synthase of claim 1, wherein the at least one modified amino acid residue is selected from the group consisting of an asparagine, a cysteine, a glycine, a lysine, a phenylalanine, a threonine, and combinations thereof.

5. The functionally active hyaluronan synthase of claim 1, wherein the functionally active hyaluronan synthase having an altered enzymatic activity has an amino acid sequence essentially as set forth in at least one of SEQ ID NOS:93-160.

6. The functionally active hyaluronan synthase of claim 1, wherein the functionally active hyaluronan synthase having an altered enzymatic activity has an increased enzymatic activity when compared to the corresponding functionally active native hyaluronan synthase.

7. The functionally active hyaluronan synthase of claim 1, wherein the functionally active hyaluronan synthase having an altered enzymatic activity has a decreased enzymatic activity when compared to the corresponding functionally active native hyaluronan synthase.

8. The functionally active hyaluronan synthase of claim 1, wherein the functionally active hyaluronan synthase having an altered enzymatic activity produces hyaluronic acid having an average molecular mass that is less than an average molecular mass of hyaluronic acid produced by the corresponding functionally active native hyaluronan synthase.

9. The functionally active hyaluronan synthase of claim 1, wherein the functionally active hyaluronan synthase having an altered enzymatic activity produces hyaluronic acid having an average molecular mass that is greater than an average molecular mass of hyaluronic acid produced by the corresponding functionally active native hyaluronan synthase.

10. A recombinant host cell having a functionally active hyaluronan synthase having an altered enzymatic activity as compared to a corresponding functionally active native hyaluronan synthase incorporated therein such that the host cell is capable of producing hyaluronan, wherein the functionally active hyaluronan synthase has at least one modified amino acid residue therein as compared to the corresponding functionally active native hyaluronan synthase.

11. The recombinant host cell of claim 10, wherein the corresponding functionally active native hyaluronan synthase is selected from the group consisting of spHAS, seHAS, suHAS and pmHAS.

12. The recombinant host cell of claim 11, wherein the corresponding functionally active native hyaluronan synthase has an amino acid sequence essentially as set forth in at least one of SEQ ID NOS:2, 4, 6, 8, 10, 12 and 14.

13. The recombinant host cell of claim 10, wherein the at least one modified amino acid residue is selected from the group consisting of an asparagine, a cysteine, a glycine, a lysine, a phenylalanine, a threonine, and combinations thereof.

14. The recombinant host cell of claim 10, wherein the functionally active hyaluronan synthase having an altered enzymatic activity has an amino acid sequence essentially as set forth in at least one of SEQ ID NOS:93-160.

15. The recombinant host cell of claim 10, wherein the functionally active hyaluronan synthase having an altered enzymatic activity has an increased enzymatic activity when compared to the corresponding functionally active native hyaluronan synthase.

16. The recombinant host cell of claim 10, wherein the functionally active hyaluronan synthase having an altered enzymatic activity has a decreased enzymatic activity when compared to the corresponding functionally active native hyaluronan synthase.

17. The recombinant host cell of claim 10, wherein the functionally active hyaluronan synthase having an altered enzymatic activity produces hyaluronic acid having an average molecular mass that is less than an average molecular mass of hyaluronic acid produced by the corresponding functionally active native hyaluronan synthase.

18. The recombinant host cell of claim 10, wherein the functionally active hyaluronan synthase having an altered enzymatic activity produces hyaluronic acid having an average molecular mass that is greater than an average molecular mass of hyaluronic acid produced by the corresponding functionally active native hyaluronan synthase.

19. The recombinant host cell of claim 10, wherein the recombinant host cell is a Bacillus cell.

20. The recombinant host cell of claim 19, wherein the recombinant host cell is selected from the group consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus metaterium, Bacillus pumilus, Bacillus stearothermophilus, Bacillus subtilis and Bacillus thuringienisis.

21. A method of providing a functionally active hyaluronan synthase having an altered enzymatic activity as compared to a corresponding functionally active native hyaluronan synthase, comprising the steps of:
providing a hyaluronan synthase; and
modifying at least one amino acid residue of the hyaluronan synthase to provide a functionally active hyaluronan synthase having an altered enzymatic activity.

22. The method of claim 21, wherein the corresponding functionally active native hyaluronan synthase is selected from the group consisting of spHAS, seHAS, suHAS and pmHAS.

23. The method of claim 22, wherein the corresponding functionally active native hyaluronan synthase has an amino acid sequence essentially as set forth in at least one of SEQ ID NOS:2, 4, 6, 8, 10, 12 and 14.

24. The method of claim 21 wherein, in the step of modifying at least one amino acid residue, the at least one modified amino acid residue is selected from the group consisting of an asparagine, a cysteine, a glycine, a lysine, a phenylalanine, a threonine, and combinations thereof.

25. The method of claim 21, wherein the functionally active hyaluronan synthase having an altered enzymatic activity has an amino acid sequence essentially as set forth in at least one of SEQ ID NOS:93-160.

26. The method of claim 21, wherein the functionally active hyaluronan synthase having an altered enzymatic activity has an increased enzymatic activity when compared to the corresponding functionally active native hyaluronan synthase.

27. The method of claim 21, wherein the functionally active hyaluronan synthase having an altered enzymatic activity has a decreased enzymatic activity when compared to the corresponding functionally active native hyaluronan synthase.

28. The method of claim 21, wherein the functionally active hyaluronan synthase having an altered enzymatic activity produces hyaluronic acid having an average molecular mass that is less than an average molecular mass of hyaluronic acid produced by the corresponding functionally active native hyaluronan synthase.

29. The method of claim 21, wherein the functionally active hyaluronan synthase having an altered enzymatic activity produces hyaluronic acid having an average molecular mass that is greater than an average molecular mass of hyaluronic acid produced by the corresponding functionally active native hyaluronan synthase.

30. A method for producing hyaluronic acid, comprising the steps of:
providing a recombinant host cell having at least one expression construct comprising a hyaluronan synthase gene encoding a functionally active hyaluronan synthase incorporated therein such that the host cell is capable of producing hyaluronan, wherein the functionally active hyaluronan synthase has at least one modified amino acid residue therein as compared to a corresponding functionally active native hyaluronan synthase such that the functionally active hyaluronan synthase has an altered enzymatic activity as compared to the corresponding functionally active native hyaluronan synthase; and
culturing the host cell under conditions appropriate for the production of hyaluronic acid.

31. The method of claim 30, wherein the corresponding functionally active native hyaluronan synthase is selected from the group consisting of spHAS, seHAS, suHAS and pmHAS.

32. The method of claim 31, wherein the corresponding functionally active native hyaluronan synthase has an amino acid sequence essentially as set forth in at least one of SEQ ID NOS:2, 4, 6, 8, 10, 12 and 14.

33. The method of claim 30, wherein the at least one modified amino acid residue is selected from the group consisting of an asparagine, a cysteine, a glycine, a lysine, a phenylalanine, a threonine, and combinations thereof.

34. The method of claim 30, wherein the functionally active hyaluronan synthase having an altered enzymatic activity has an amino acid sequence essentially as set forth in at least one of SEQ ID NOS:93-160.

35. The method of claim 30, wherein the functionally active hyaluronan synthase having an altered enzymatic activity has an increased enzymatic activity when compared to the corresponding functionally active native hyaluronan synthase.

36. The method of claim 30, wherein the functionally active hyaluronan synthase having an altered enzymatic activity has a decreased enzymatic activity when compared to the corresponding functionally active native hyaluronan synthase.

37. The method of claim 30, wherein the functionally active hyaluronan synthase having an altered enzymatic activity produces hyaluronic acid having an average molecular mass that is less than an average molecular mass of hyaluronic acid produced by the corresponding functionally active native hyaluronan synthase.

38. The method of claim 30, wherein the functionally active hyaluronan synthase having an altered enzymatic activity produces hyaluronic acid having an average molecular mass that is greater than an average molecular mass of hyaluronic acid produced by the corresponding functionally active native hyaluronan synthase.

39. The method of claim 30, further comprising the step of separating the hyaluronic acid from the host cell.

40. The method of claim 30, wherein the recombinant host cell further comprises at least one gene encoding an enzyme for synthesis of a hyaluronic acid sugar precursor.

41. The method of claim 30, wherein the at least one gene encoding an enzyme for synthesis of a hyaluronic acid sugar precursor is selected from the group consisting of a pyrophosphorylase, a transferase, a mutase, a dehydrogenase, an epimerase capable of increasing production of UDP-GlcNAc or UDP-GlcUA, and combinations thereof.

42. The method of claim 30, wherein nutrients utilized for a hyaluronic acid sugar precursor biosynthetic pathway are supplied to the host cell.

43. The method of claim 30, wherein the recombinant host cell is a Bacillus cell.

44. The method of claim 43, wherein the recombinant host cell is selected from the group consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus metaterium, Bacillus pumilus, Bacillus stearothermophilus, Bacillus subtilis and Bacillus thuringienisis.
